SEQUENCE LISTING (429 sequences)

SEQ ID NO: 1 - protein, 11 aa, Artificial Sequence
  sequence is synthesized
FCXDWPXXXSC

SEQ ID NO: 2 - protein, 9 aa, Artificial Sequence
  sequence is synthesized
VCYXXXICF

SEQ ID NO: 3 - protein, 7 aa, Artificial Sequence
  sequence is synthesized
CYXPGXC

SEQ ID NO: 4 - protein, 11 aa, Artificial Sequence
  sequence is synthesized
DXCLPXWGCLW

SEQ ID NO: 5 - protein, 12 aa, Artificial Sequence
  sequence is synthesized
WCDXXLXAXDLC

SEQ ID NO: 6 - protein, 10 aa, Artificial Sequence
  sequence is synthesized
DLVXLGLECW

SEQ ID NO: 7 - protein, 11 aa, Artificial Sequence
  sequence is synthesized
DLCLRDWGCLW

SEQ ID NO: 8 - protein, 11 aa, Artificial Sequence
  sequence is synthesized
DICLPRWGCLW

SEQ ID NO: 9 - protein, 15 aa, Artificial Sequence
  sequence is synthesized
MEDICLPRWGCLWGD

SEQ ID NO: 10 - protein, 20 aa, Artificial Sequence
  sequence is synthesized
QRLMEDICLPRWGCLWEDDE

SEQ ID NO: 11 - protein, 20 aa, Artificial Sequence
  sequence is synthesized
QGLIGDICLPRWGCLWGDSV

SEQ ID NO: 12 - protein, 21 aa, Artificial Sequence
  sequence is synthesized
QGLIGDICLPRWGCLWGDSVK

SEQ ID NO: 13 - protein, 15 aa, Artificial Sequence
  sequence is synthesized
EDICLPRWGCLWEDD

SEQ ID NO: 14 - protein, 18 aa, Artificial Sequence
  sequence is synthesized
RLMEDICLPRWGCLWEDD

SEQ ID NO: 15 - protein, 16 aa, Artificial Sequence
  sequence is synthesized
MEDICLPRWGCLWEDD

SEQ ID NO: 16 - protein, 15 aa, Artificial Sequence
  sequence is synthesized
MEDICLPRWGCLWED

SEQ ID NO: 17 - protein, 18 aa, Artificial Sequence
  sequence is synthesized
RLMEDICLARWGCLWEDD

SEQ ID NO: 18 - protein, 20 aa, Artificial Sequence
  sequence is synthesized
EVRSFCTRWPAEKSCKPLRG

SEQ ID NO: 19 - protein, 20 aa, Artificial Sequence
  sequence is synthesized
RAPESFVCYWETICFERSEQ

SEQ ID NO: 20 - protein, 11 aa, Artificial Sequence
  sequence is synthesized
EMCYFPGICWM

SEQ ID NO: 21 - protein, 10 aa, Artificial Sequence
  sequence is synthesized
CXXGPXXXXC

SEQ ID NO: 22 - protein, 18 aa, Artificial Sequence
  sequence is synthesized
XXXXCXXGPXXXXCXXXX

SEQ ID NO: 23 - protein, 27 aa, Artificial Sequence
  sequence is synthesized
CXXXXXXCXXXXXXCCXXXCXXXXXXC

SEQ ID NO: 24 - protein, 13 aa, Artificial Sequence
  sequence is synthesized
CCXXXCXXXXXXC

SEQ ID NO: 25 - protein, 19 aa, Artificial Sequence
  sequence is synthesized
CCXXXXXCXXXXCXXXXCC

SEQ ID NO: 26 - protein, 21 aa, Artificial Sequence
  sequence is synthesized
CXCXXXXXXXCXXXCXXXXXX

SEQ ID NO: 27 - protein, 20 aa, Artificial Sequence
  sequence is synthesized
GENWCDSTLMAYDLCGQVNM

SEQ ID NO: 28 - protein, 20 aa, Artificial Sequence
  sequence is synthesized
MDELAFYCGIWECLMHQEQK

SEQ ID NO: 29 - protein, 10 aa, Artificial Sequence
  sequence is synthesized
DLCDVDFCWF

SEQ ID NO: 30 - protein, 16 aa, Artificial Sequence
  sequence is synthesized
KSCSELHWLLVEECLF

SEQ ID NO: 31 - protein, 20 aa, Artificial Sequence
  sequence is synthesized
EVRSFCTDWPAEKSCKPLRG

SEQ ID NO: 32 - protein, 27 aa, Artificial Sequence
  sequence is synthesized
CEVALDACRGGESGCCRHICELIRQLC

SEQ ID NO: 33 - protein, 20 aa, Artificial Sequence
  sequence is synthesized
RNEDPCVVLLEMGLECWEGV

SEQ ID NO: 34 - protein, 15 aa, Artificial Sequence
  sequence is synthesized
DTCVDLVRLGLECWG

SEQ ID NO: 35 - protein, 20 aa, Artificial Sequence
  sequence is synthesized
QRQMVDFCLPQWGCLWGDGF

SEQ ID NO: 36 - protein, 21 aa, Artificial Sequence
  sequence is synthesized
CGCVDVSDWDCWSECLWSHGA

SEQ ID NO: 37 - protein, 20 aa, Artificial Sequence
  sequence is synthesized
GEDWCDSTLLAFDLCGEGAR

SEQ ID NO: 38 - protein, 20 aa, Artificial Sequence
  sequence is synthesized
GENWCDWVLLAYDLCGEDNT

SEQ ID NO: 39 - protein, 20 aa, Artificial Sequence
  sequence is synthesized
MELWCDSTLMAYDLCGDFNM

SEQ ID NO: 40 - protein, 20 aa, Artificial Sequence
  sequence is synthesized
EVRSFCTDWPAHYSCTSLQG

SEQ ID NO: 41 - protein, 19 aa, Artificial Sequence
  sequence is synthesized
GRSFCMDWPAHKSCTPLML

SEQ ID NO: 42 - protein, 20 aa, Artificial Sequence
  sequence is synthesized
GVRTFCQDWPAHNSCKLLRG

SEQ ID NO: 43 - protein, 20 aa, Artificial Sequence
  sequence is synthesized
QTRSFCADWPRHESCKPLRG

SEQ ID NO: 44 - protein, 15 aa, Artificial Sequence
  sequence is synthesized
RRTCDWPHNSCKLRG

SEQ ID NO: 45 - protein, 20 aa, Artificial Sequence
  sequence is synthesized
RAAESSVCYWPGICFDRTEQ

SEQ ID NO: 46 - protein, 20 aa, Artificial Sequence
  sequence is synthesized
MEPSRSVCYAEGICFDRGEQ

SEQ ID NO: 47 - protein, 20 aa, Artificial Sequence
  sequence is synthesized
REPASLVCYFEDICFVRAEA

SEQ ID NO: 48 - protein, 18 aa, Artificial Sequence
  sequence is synthesized
RGPDVCYWPSICFERSMP

SEQ ID NO: 49 - protein, 20 aa, Artificial Sequence
  sequence is synthesized
LVPERIVCYFESICYERSEL

SEQ ID NO: 50 - protein, 20 aa, Artificial Sequence
  sequence is synthesized
RMPASLPCYWETICYESSEQ

SEQ ID NO: 51 - protein, 20 aa, Artificial Sequence
  sequence is synthesized
RTAESLVCYWPGICFAQSER

SEQ ID NO: 52 - protein, 20 aa, Artificial Sequence
  sequence is synthesized
RAPERWVCYWEGICFDRYEQ

SEQ ID NO: 53 - protein, 11 aa, Artificial Sequence
  sequence is synthesized
EICYFPGICWI

SEQ ID NO: 54 - protein, 11 aa, Artificial Sequence
  sequence is synthesized
ELCYFPGICWT

SEQ ID NO: 55 - protein, 11 aa, Artificial Sequence
  sequence is synthesized
DICYIPGICWM

SEQ ID NO: 56 - protein, 11 aa, Artificial Sequence
  sequence is synthesized
KLCYFPGICWS

SEQ ID NO: 57 - protein, 11 aa, Artificial Sequence
  sequence is synthesized
DLCYFPGICWM

SEQ ID NO: 58 - protein, 11 aa, Artificial Sequence
  sequence is synthesized
GMCYFPGICWA

SEQ ID NO: 59 - protein, 11 aa, Artificial Sequence
  sequence is synthesized
EMCYFPGICWS

SEQ ID NO: 60 - protein, 11 aa, Artificial Sequence
  sequence is synthesized
EMCYFPGICWT

SEQ ID NO: 61 - protein, 11 aa, Artificial Sequence
  sequence is synthesized
KTCYFPGICWM

SEQ ID NO: 62 - protein, 11 aa, Artificial Sequence
  sequence is synthesized
KVCYFPGICWM

SEQ ID NO: 63 - protein, 11 aa, Artificial Sequence
  sequence is synthesized
DVCYFPGICWM

SEQ ID NO: 64 - protein, 11 aa, Artificial Sequence
  sequence is synthesized
EICYFPGICWM

SEQ ID NO: 65 - protein, 11 aa, Artificial Sequence
  sequence is synthesized
ALCYFPGICWM

SEQ ID NO: 66 - protein, 11 aa, Artificial Sequence
  sequence is synthesized
ELCYFPGICWP

SEQ ID NO: 67 - protein, 11 aa, Artificial Sequence
  sequence is synthesized
ELCYFPGICWM

SEQ ID NO: 68 - protein, 11 aa, Artificial Sequence
  sequence is synthesized
DMCYFPGICWL

SEQ ID NO: 69 - protein, 11 aa, Artificial Sequence
  sequence is synthesized
DMCYFPGICFN

SEQ ID NO: 70 - protein, 11 aa, Artificial Sequence
  sequence is synthesized
ETCYFPGICWL

SEQ ID NO: 71 - protein, 11 aa, Artificial Sequence
  sequence is synthesized
EVCYFPGICWF

SEQ ID NO: 72 - protein, 11 aa, Artificial Sequence
  sequence is synthesized
EVCYFPGICWE

SEQ ID NO: 73 - protein, 11 aa, Artificial Sequence
  sequence is synthesized
EVCYFPGICWM

SEQ ID NO: 74 - protein, 15 aa, Artificial Sequence
  sequence is synthesized
LAEMCYFPGICWMSA

SEQ ID NO: 75 - protein, 15 aa, Artificial Sequence
  sequence is synthesized
GGEICYFPGICRVLP

SEQ ID NO: 76 - protein, 15 aa, Artificial Sequence
  sequence is synthesized
EHDMCYFPGICWIAD

SEQ ID NO: 77 - protein, 15 aa, Artificial Sequence
  sequence is synthesized
VQEVCYFPGICWMQE

SEQ ID NO: 78 - protein, 15 aa, Artificial Sequence
  sequence is synthesized
SREVCYYPGICWNGA

SEQ ID NO: 79 - protein, 15 aa, Artificial Sequence
  sequence is synthesized
DSEVCYFPGICWSGT

SEQ ID NO: 80 - protein, 15 aa, Artificial Sequence
  sequence is synthesized
GTEVCYFPGICWGGG

SEQ ID NO: 81 - protein, 15 aa, Artificial Sequence
  sequence is synthesized
SYAPCYFPGICWMGN

SEQ ID NO: 82 - protein, 15 aa, Artificial Sequence
  sequence is synthesized
HAEICYFPGICWTER

SEQ ID NO: 83 - protein, 15 aa, Artificial Sequence
  sequence is synthesized
NDEICYFPGVCWKSG

SEQ ID NO: 84 - protein, 15 aa, Artificial Sequence
  sequence is synthesized
RDTVCYFPGICWMAS

SEQ ID NO: 85 - protein, 15 aa, Artificial Sequence
  sequence is synthesized
VRDMCYFPGICWKSE

SEQ ID NO: 86 - protein, 15 aa, Artificial Sequence
  sequence is synthesized
ASEICYFPGICWMVE

SEQ ID NO: 87 - protein, 15 aa, Artificial Sequence
  sequence is synthesized
QTELCYFPGICWNES

SEQ ID NO: 88 - protein, 15 aa, Artificial Sequence
  sequence is synthesized
TTEMCYFPGICWKTE

SEQ ID NO: 89 - protein, 15 aa, Artificial Sequence
  sequence is synthesized
KTEICYFPGICWMSG

SEQ ID NO: 90 - protein, 8 aa, Artificial Sequence
  sequence is synthesized
QCFPGWVK

SEQ ID NO: 91 - protein, 15 aa, Artificial Sequence
  sequence is synthesized
IVEMCYYPGICWISP

SEQ ID NO: 92 - protein, 15 aa, Artificial Sequence
  sequence is synthesized
SGAICYVPGICWTHA

SEQ ID NO: 93 - protein, 20 aa, Artificial Sequence
  sequence is synthesized
QRHPEDICLPRWGCLWGDDD

SEQ ID NO: 94 - protein, 20 aa, Artificial Sequence
  sequence is synthesized
NRQMEDICLPQWGCLWGDDF

SEQ ID NO: 95 - protein, 20 aa, Artificial Sequence
  sequence is synthesized
QRLMEDICLPRWGCLWGDRF

SEQ ID NO: 96 - protein, 20 aa, Artificial Sequence
  sequence is synthesized
QWHMEDICLPQWGCLWGDVL

SEQ ID NO: 97 - protein, 20 aa, Artificial Sequence
  sequence is synthesized
QWQMENVCLPKWGCLWEELD

SEQ ID NO: 98 - protein, 20 aa, Artificial Sequence
  sequence is synthesized
LWAMEDICLPKWGCLWEDDF

SEQ ID NO: 99 - protein, 20 aa, Artificial Sequence
  sequence is synthesized
LRLMDNICLPRWGCLWDDGF

SEQ ID NO: 100 - protein, 20 aa, Artificial Sequence
  sequence is synthesized
HSQMEDICLPRWGCLWGDEL

SEQ ID NO: 101 - protein, 20 aa, Artificial Sequence
  sequence is synthesized
QWQVMDICLPRWGCLWADEY

SEQ ID NO: 102 - protein, 20 aa, Artificial Sequence
  sequence is synthesized
HRLVEDICLPRWGCLWGNDF

SEQ ID NO: 103 - protein, 20 aa, Artificial Sequence
  sequence is synthesized
QMHMMDICLPKWGCLWGDTS

SEQ ID NO: 104 - protein, 20 aa, Artificial Sequence
  sequence is synthesized
LRIFEDICLPKWGCLWGEGF

SEQ ID NO: 105 - protein, 20 aa, Artificial Sequence
  sequence is synthesized
QSYMEDICLPRWGCLSDDAS

SEQ ID NO: 106 - protein, 20 aa, Artificial Sequence
  sequence is synthesized
QGDFWDICLPRWGCLSGEGY

SEQ ID NO: 107 - protein, 20 aa, Artificial Sequence
  sequence is synthesized
RWQTEDVCLPKWGCLFGDGV

SEQ ID NO: 108 - protein, 20 aa, Artificial Sequence
  sequence is synthesized
LIFMEDVCLPQWGCLWEDGV

SEQ ID NO: 109 - protein, 20 aa, Artificial Sequence
  sequence is synthesized
QRDMGDICLPRWGCLWEDGV

SEQ ID NO: 110 - protein, 20 aa, Artificial Sequence
  sequence is synthesized
QRHMMDFCLPKWGCLWGDGY

SEQ ID NO: 111 - protein, 20 aa, Artificial Sequence
  sequence is synthesized
QRPIMDFCLPKWGCLWEDGF

SEQ ID NO: 112 - protein, 20 aa, Artificial Sequence
  sequence is synthesized
ERQMVDFCLPKWGCLWGDGF

SEQ ID NO: 113 - protein, 20 aa, Artificial Sequence
  sequence is synthesized
QGYMVDFCLPRWGCLWGDAN

SEQ ID NO: 114 - protein, 20 aa, Artificial Sequence
  sequence is synthesized
KMGRVDFCLPKWGCLWGDEL

SEQ ID NO: 115 - protein, 20 aa, Artificial Sequence
  sequence is synthesized
QSQLEDFCLPKWGCLWGDGF

SEQ ID NO: 116 - protein, 20 aa, Artificial Sequence
  sequence is synthesized
QGGMGDFCLPQWGCLWGEDL

SEQ ID NO: 117 - protein, 20 aa, Artificial Sequence
  sequence is synthesized
QRLMWEICLPLWGCLWGDGL

SEQ ID NO: 118 - protein, 20 aa, Artificial Sequence
  sequence is synthesized
QRQIMDFCLPHWGCLWGDGF

SEQ ID NO: 119 - protein, 20 aa, Artificial Sequence
  sequence is synthesized
GRQVVDFCLPKWGCLWEEGL

SEQ ID NO: 120 - protein, 20 aa, Artificial Sequence
  sequence is synthesized
QMQMSDFCLPQWGCLWGDGY

SEQ ID NO: 121 - protein, 20 aa, Artificial Sequence
  sequence is synthesized
KSRMGDFCLPEWGCLWGDEL

SEQ ID NO: 122 - protein, 20 aa, Artificial Sequence
  sequence is synthesized
ERQMEDFCLPQWGCLWGDGV

SEQ ID NO: 123 - protein, 20 aa, Artificial Sequence
  sequence is synthesized
QRQVVDFCLPQWGCLWGDGS

SEQ ID NO: 124 - protein, 11 aa, Artificial Sequence
  sequence is synthesized
DICLPEWGCLW

SEQ ID NO: 125 - protein, 11 aa, Artificial Sequence
  sequence is synthesized
DICLPVWGCLW

SEQ ID NO: 126 - protein, 11 aa, Artificial Sequence
  sequence is synthesized
DLCLPEWGCLW

SEQ ID NO: 127 - protein, 11 aa, Artificial Sequence
  sequence is synthesized
DLCLPKWGCLW

SEQ ID NO: 128 - protein, 11 aa, Artificial Sequence
  sequence is synthesized
DLCLPVWGCLW

SEQ ID NO: 129 - protein, 11 aa, Artificial Sequence
  sequence is synthesized
DICLPAWGCLW

SEQ ID NO: 130 - protein, 11 aa, Artificial Sequence
  sequence is synthesized
DICLPDWGCLW

SEQ ID NO: 131 - protein, 11 aa, Artificial Sequence
  sequence is synthesized
DICLERWGCLW

SEQ ID NO: 132 - protein, 15 aa, Artificial Sequence
  sequence is synthesized
EWDVCLPHWGCLWDG

SEQ ID NO: 133 - protein, 15 aa, Artificial Sequence
  sequence is synthesized
WDDICFRDWGCLWGS

SEQ ID NO: 134 - protein, 15 aa, Artificial Sequence
  sequence is synthesized
MDDICLHHWGCLWDE

SEQ ID NO: 135 - protein, 15 aa, Artificial Sequence
  sequence is synthesized
MDDLCLPNWGCLWGD

SEQ ID NO: 136 - protein, 15 aa, Artificial Sequence
  sequence is synthesized
FEDFCLPNWGCLWGS

SEQ ID NO: 137 - protein, 15 aa, Artificial Sequence
  sequence is synthesized
FEDLCVVRWGCLWGD

SEQ ID NO: 138 - protein, 15 aa, Artificial Sequence
  sequence is synthesized
WEDLCLPDWGCLWED

SEQ ID NO: 139 - protein, 15 aa, Artificial Sequence
  sequence is synthesized
SEDFCLPVWGCLWED

SEQ ID NO: 140 - protein, 15 aa, Artificial Sequence
  sequence is synthesized
DFDLCLPDWGCLWDD

SEQ ID NO: 141 - protein, 15 aa, Artificial Sequence
  sequence is synthesized
NWDLCFPDWGCLWDD

SEQ ID NO: 142 - protein, 15 aa, Artificial Sequence
  sequence is synthesized
EEDLCLPVWGCLWGA

SEQ ID NO: 143 - protein, 15 aa, Artificial Sequence
  sequence is synthesized
EEDVCLPVWGCLWEG

SEQ ID NO: 144 - protein, 15 aa, Artificial Sequence
  sequence is synthesized
MFDLCLPKWGCLWGN

SEQ ID NO: 145 - protein, 15 aa, Artificial Sequence
  sequence is synthesized
EFDLCLPTWGCLWED

SEQ ID NO: 146 - protein, 15 aa, Artificial Sequence
  sequence is synthesized
MWDVCFPDWGCLWDV

SEQ ID NO: 147 - protein, 15 aa, Artificial Sequence
  sequence is synthesized
EWDVCFPAWGCLWDQ

SEQ ID NO: 148 - protein, 15 aa, Artificial Sequence
  sequence is synthesized
VWDLCLPQWGCLWDE

SEQ ID NO: 149 - protein, 15 aa, Artificial Sequence
  sequence is synthesized
DTCADLVRLGLECWA

SEQ ID NO: 150 - protein, 15 aa, Artificial Sequence
  sequence is synthesized
NTCADLVRLGLECWA

SEQ ID NO: 151 - protein, 15 aa, Artificial Sequence
  sequence is synthesized
DTCDDLVQLGLECWA

SEQ ID NO: 152 - protein, 15 aa, Artificial Sequence
  sequence is synthesized
DTCEDLVRLGLECWA

SEQ ID NO: 153 - protein, 15 aa, Artificial Sequence
  sequence is synthesized
DSCGDLLRLGLECWA

SEQ ID NO: 154 - protein, 15 aa, Artificial Sequence
  sequence is synthesized
DTCSDLVGLGLECWA

SEQ ID NO: 155 - protein, 13 aa, Artificial Sequence
  sequence is synthesized
XDXCLPXWGCLWX

SEQ ID NO: 156 - protein, 13 aa, Artificial Sequence
  sequence is synthesized
XDXCLPXWGCLWX

SEQ ID NO: 157 - protein, 20 aa, Artificial Sequence
  sequence is synthesized
AAQVGDICLPRWGCLWSEYA

SEQ ID NO: 158 - protein, 20 aa, Artificial Sequence
  sequence is synthesized
AGWAADVCLPRWGCLWEEDV

SEQ ID NO: 159 - protein, 20 aa, Artificial Sequence
  sequence is synthesized
ASVVDDICLPVWGCLWGEDI

SEQ ID NO: 160 - protein, 20 aa, Artificial Sequence
  sequence is synthesized
ATMEDDICLPRWGCLWGAEE

SEQ ID NO: 161 - protein, 20 aa, Artificial Sequence
  sequence is synthesized
DEDFEDYCLPPWGCLWGSSM

SEQ ID NO: 162 - protein, 20 aa, Artificial Sequence
  sequence is synthesized
EGTWDDFCLPRWGCLWLGER

SEQ ID NO: 163 - protein, 20 aa, Artificial Sequence
  sequence is synthesized
ERWEGDVCLPRWGCLWGESG

SEQ ID NO: 164 - protein, 20 aa, Artificial Sequence
  sequence is synthesized
GDWMHDICLPKWGCLWDEKA

SEQ ID NO: 165 - protein, 20 aa, Artificial Sequence
  sequence is synthesized
GIEWGDTCLPKWGCLWRVEG

SEQ ID NO: 166 - protein, 20 aa, Artificial Sequence
  sequence is synthesized
GQQGEDVCLPVWGCLWDTSS

SEQ ID NO: 167 - protein, 20 aa, Artificial Sequence
  sequence is synthesized
GRYPMDLCLPRWGCLWEDSA

SEQ ID NO: 168 - protein, 20 aa, Artificial Sequence
  sequence is synthesized
GSAGDDLCLPRWGCLWERGA

SEQ ID NO: 169 - protein, 20 aa, Artificial Sequence
  sequence is synthesized
HASDWDVCLPGWGCLWEEDD

SEQ ID NO: 170 - protein, 20 aa, Artificial Sequence
  sequence is synthesized
LGVTHDTCLPRWGCLWDEVG

SEQ ID NO: 171 - protein, 20 aa, Artificial Sequence
  sequence is synthesized
LVWEEDFCLPKWGCLWGAED

SEQ ID NO: 172 - protein, 20 aa, Artificial Sequence
  sequence is synthesized
NVGWNDICLPRWGCLWAQES

SEQ ID NO: 173 - protein, 20 aa, Artificial Sequence
  sequence is synthesized
QGVEWDVCLPQWGCLWTREV

SEQ ID NO: 174 - protein, 20 aa, Artificial Sequence
  sequence is synthesized
RLDAWDICLPQWGCLWEEPS

SEQ ID NO: 175 - protein, 20 aa, Artificial Sequence
  sequence is synthesized
SEAPGDYCLPRWGCLWAQEK

SEQ ID NO: 176 - protein, 20 aa, Artificial Sequence
  sequence is synthesized
TAMDEDVCLPRWGCLWGSGS

SEQ ID NO: 177 - protein, 20 aa, Artificial Sequence
  sequence is synthesized
TEIGQDFCLPRWGCLWVPGT

SEQ ID NO: 178 - protein, 20 aa, Artificial Sequence
  sequence is synthesized
TLGWPDFCLPKWGCLWRESD

SEQ ID NO: 179 - protein, 20 aa, Artificial Sequence
  sequence is synthesized
TLSNQDICLPGWGCLWGGIN

SEQ ID NO: 180 - protein, 20 aa, Artificial Sequence
  sequence is synthesized
TSTGGDLCLPRWGCLWDSSE

SEQ ID NO: 181 - protein, 20 aa, Artificial Sequence
  sequence is synthesized
VSEMDDICLPLWGCLWADAP

SEQ ID NO: 182 - protein, 20 aa, Artificial Sequence
  sequence is synthesized
VSEWEDICLPSWGCLWETQD

SEQ ID NO: 183 - protein, 20 aa, Artificial Sequence
  sequence is synthesized
VVGDGDFCLPKWGCLWDQAR

SEQ ID NO: 184 - protein, 20 aa, Artificial Sequence
  sequence is synthesized
VVWDDDVCLPRWGCLWEEYG

SEQ ID NO: 185 - protein, 20 aa, Artificial Sequence
  sequence is synthesized
WSDSDDVCLPRWGCLWGNVA

SEQ ID NO: 186 - protein, 20 aa, Artificial Sequence
  sequence is synthesized
WVEEGDICLPRWGCLWESVE

SEQ ID NO: 187 - protein, 20 aa, Artificial Sequence
  sequence is synthesized
AQAMGDICLPRWGCLWEAEI

SEQ ID NO: 188 - protein, 20 aa, Artificial Sequence
  sequence is synthesized
ASDRGDLCLPYWGCLWGPDG

SEQ ID NO: 189 - protein, 20 aa, Artificial Sequence
  sequence is synthesized
ASDPGDVCLPRWGCLWGESF

SEQ ID NO: 190 - protein, 20 aa, Artificial Sequence
  sequence is synthesized
ASNWEDVCLPRWGCLWGERN

SEQ ID NO: 191 - protein, 20 aa, Artificial Sequence
  sequence is synthesized
ASTPRDICLPRWGCLWSEDA

SEQ ID NO: 192 - protein, 20 aa, Artificial Sequence
  sequence is synthesized
DGEEGDLCLPRWGCLWALEH

SEQ ID NO: 193 - protein, 20 aa, Artificial Sequence
  sequence is synthesized
EGEEVDICLPQWGCLWGYPV

SEQ ID NO: 194 - protein, 20 aa, Artificial Sequence
  sequence is synthesized
EVGDLDLCLPRWGCLWGNDK

SEQ ID NO: 195 - protein, 20 aa, Artificial Sequence
  sequence is synthesized
FRDGEDFCLPQWGCLWADTS

SEQ ID NO: 196 - protein, 20 aa, Artificial Sequence
  sequence is synthesized
GDMVNDFCLPRWGCLWGSEN

SEQ ID NO: 197 - protein, 20 aa, Artificial Sequence
  sequence is synthesized
GRMGTDLCLPRWGCLWGEVE

SEQ ID NO: 198 - protein, 20 aa, Artificial Sequence
  sequence is synthesized
HEWERDICLPRWGCLWRDGD

SEQ ID NO: 199 - protein, 20 aa, Artificial Sequence
  sequence is synthesized
KKVSGDICLPIWGCLWDNDY

SEQ ID NO: 200 - protein, 20 aa, Artificial Sequence
  sequence is synthesized
LLESDDICLPRWGCLWHEDG

SEQ ID NO: 201 - protein, 20 aa, Artificial Sequence
  sequence is synthesized
MQAESDFCLPHWGCLWDEGT

SEQ ID NO: 202 - protein, 20 aa, Artificial Sequence
  sequence is synthesized
MQGPLDICLPRWGCLWGGVD

SEQ ID NO: 203 - protein, 20 aa, Artificial Sequence
  sequence is synthesized
QMPLEDICLPRWGCLWEGRE

SEQ ID NO: 204 - protein, 20 aa, Artificial Sequence
  sequence is synthesized
REEWGDLCLPTWGCLWETKK

SEQ ID NO: 205 - protein, 20 aa, Artificial Sequence
  sequence is synthesized
RVWTEDVCLPRWGCLWSEGN

SEQ ID NO: 206 - protein, 20 aa, Artificial Sequence
  sequence is synthesized
SIREYDVCLPKWGCLWEPSA

SEQ ID NO: 207 - protein, 20 aa, Artificial Sequence
  sequence is synthesized
SPTEWDMCLPKWGCLWGDAL

SEQ ID NO: 208 - protein, 20 aa, Artificial Sequence
  sequence is synthesized
SSGLEDICLPNWGCLWADGS

SEQ ID NO: 209 - protein, 20 aa, Artificial Sequence
  sequence is synthesized
SVGWGDICLPVWGCLWGEGG

SEQ ID NO: 210 - protein, 20 aa, Artificial Sequence
  sequence is synthesized
TEENWDLCLPRWGCLWGDDW

SEQ ID NO: 211 - protein, 20 aa, Artificial Sequence
  sequence is synthesized
TSGSDDICLPVWGCLWGEDS

SEQ ID NO: 212 - protein, 19 aa, Artificial Sequence
  sequence is synthesized
TWPGDLCLPRWGCLWEAES

SEQ ID NO: 213 - protein, 20 aa, Artificial Sequence
  sequence is synthesized
WDHELDFCLPVWGCLWAEDV

SEQ ID NO: 214 - protein, 20 aa, Artificial Sequence
  sequence is synthesized
WTESEDICLPGWGCLWGPEV

SEQ ID NO: 215 - protein, 20 aa, Artificial Sequence
  sequence is synthesized
WVPFEDVCLPRWGCLWSSYQ

SEQ ID NO: 216 - protein, 18 aa, Artificial Sequence
  sequence is synthesized
EEDSDICLPRWGCLWNTS

SEQ ID NO: 217 - protein, 18 aa, Artificial Sequence
  sequence is synthesized
EGYWDLCLPRWGCLWELE

SEQ ID NO: 218 - protein, 18 aa, Artificial Sequence
  sequence is synthesized
ELGEDLCLPRWGCLWGSE

SEQ ID NO: 219 - protein, 18 aa, Artificial Sequence
  sequence is synthesized
ETWSDVCLPRWGCLWGAS

SEQ ID NO: 220 - protein, 18 aa, Artificial Sequence
  sequence is synthesized
GDYVDLCLPGWGCLWEDG

SEQ ID NO: 221 - protein, 18 aa, Artificial Sequence
  sequence is synthesized
GVLDDICLPRWGCLWGPK

SEQ ID NO: 222 - protein, 18 aa, Artificial Sequence
  sequence is synthesized
HMMDDVCLPGWGCLWASE

SEQ ID NO: 223 - protein, 18 aa, Artificial Sequence
  sequence is synthesized
IDYTDLCLPAWGCLWELE

SEQ ID NO: 224 - protein, 18 aa, Artificial Sequence
  sequence is synthesized
IEHEDLCLPRWGCLWAVD

SEQ ID NO: 225 - protein, 18 aa, Artificial Sequence
  sequence is synthesized
ISEWDLCLPRWGCLWDRS

SEQ ID NO: 226 - protein, 18 aa, Artificial Sequence
  sequence is synthesized
ISWADVCLPKWGCLWGKD

SEQ ID NO: 227 - protein, 18 aa, Artificial Sequence
  sequence is synthesized
ISWGDLCLPRWGCLWEGS

SEQ ID NO: 228 - protein, 18 aa, Artificial Sequence
  sequence is synthesized
KLWDDICLPRWGCLWSPL

SEQ ID NO: 229 - protein, 18 aa, Artificial Sequence
  sequence is synthesized
LAWPDVCLPRWGCLWGGM

SEQ ID NO: 230 - protein, 18 aa, Artificial Sequence
  sequence is synthesized
LNESDICLPTWGCLWGVD

SEQ ID NO: 231 - protein, 18 aa, Artificial Sequence
  sequence is synthesized
LPEQDVCLPVWGCLWDAN

SEQ ID NO: 232 - protein, 18 aa, Artificial Sequence
  sequence is synthesized
MAWGDVCLPRWGCLWAGG

SEQ ID NO: 233 - protein, 18 aa, Artificial Sequence
  sequence is synthesized
NEEWDVCLPRWGCLWGGV

SEQ ID NO: 234 - protein, 18 aa, Artificial Sequence
  sequence is synthesized
QELQDFCLPRWGCLWGVG

SEQ ID NO: 235 - protein, 18 aa, Artificial Sequence
  sequence is synthesized
QREWDVCLPRWGCLWSDV

SEQ ID NO: 236 - protein, 18 aa, Artificial Sequence
  sequence is synthesized
QRFWDTCLPRWGCLWGGD

SEQ ID NO: 237 - protein, 18 aa, Artificial Sequence
  sequence is synthesized
RVFTDVCLPRWGCLWDLG

SEQ ID NO: 238 - protein, 18 aa, Artificial Sequence
  sequence is synthesized
SGWDDVCLPVWGCLWGPS

SEQ ID NO: 239 - protein, 18 aa, Artificial Sequence
  sequence is synthesized
SSASDYCLPRWGCLWGDL

SEQ ID NO: 240 - protein, 18 aa, Artificial Sequence
  sequence is synthesized
SWQGDICLPRWGCLWGVD

SEQ ID NO: 241 - protein, 18 aa, Artificial Sequence
  sequence is synthesized
SYETDVCLPYWGCLWEDA

SEQ ID NO: 242 - protein, 18 aa, Artificial Sequence
  sequence is synthesized
SYWGDVCLPRWGCLWSEA

SEQ ID NO: 243 - protein, 18 aa, Artificial Sequence
  sequence is synthesized
TLEWDMCLPRWGCLWTEQ

SEQ ID NO: 244 - protein, 18 aa, Artificial Sequence
  sequence is synthesized
VGEFDICLPRWGCLWDAE

SEQ ID NO: 245 - protein, 18 aa, Artificial Sequence
  sequence is synthesized
VTSWDVCLPRWGCLWEED

SEQ ID NO: 246 - protein, 18 aa, Artificial Sequence
  sequence is synthesized
WLWEDLCLPKWGCLWEED

SEQ ID NO: 247 - protein, 18 aa, Artificial Sequence
  sequence is synthesized
ALFEDVCLPVWGCLWGGE

SEQ ID NO: 248 - protein, 18 aa, Artificial Sequence
  sequence is synthesized
ASEWDVCLPTWGCLWMEG

SEQ ID NO: 249 - protein, 18 aa, Artificial Sequence
  sequence is synthesized
AYSADICLPRWGCLWMSE

SEQ ID NO: 250 - protein, 18 aa, Artificial Sequence
  sequence is synthesized
EDWEDICLPQWGCLWEGM

SEQ ID NO: 251 - protein, 18 aa, Artificial Sequence
  sequence is synthesized
EDWTDLCLPAWGCLWDTE

SEQ ID NO: 252 - protein, 18 aa, Artificial Sequence
  sequence is synthesized
EEWEDLCLPRWGCLWSAE

SEQ ID NO: 253 - protein, 18 aa, Artificial Sequence
  sequence is synthesized
EFWQDICLPNWGCLWAES

SEQ ID NO: 254 - protein, 18 aa, Artificial Sequence
  sequence is synthesized
EGFSDICLPRWGCLWSQE

SEQ ID NO: 255 - protein, 18 aa, Artificial Sequence
  sequence is synthesized
ETWEDLCLPNWGCLWDLE

SEQ ID NO: 256 - protein, 18 aa, Artificial Sequence
  sequence is synthesized
GEVNDFCLPRWGCLWEGD

SEQ ID NO: 257 - protein, 18 aa, Artificial Sequence
  sequence is synthesized
GGEWDVCLPAWGCLWGEE

SEQ ID NO: 258 - protein, 18 aa, Artificial Sequence
  sequence is synthesized
KDWYDICLPRWGCLWGGE

SEQ ID NO: 259 - protein, 18 aa, Artificial Sequence
  sequence is synthesized
KLGQDICLPRWGCLWDFA

SEQ ID NO: 260 - protein, 18 aa, Artificial Sequence
  sequence is synthesized
LEEWDICLPQWGCLWREG

SEQ ID NO: 261 - protein, 18 aa, Artificial Sequence
  sequence is synthesized
LVLPDICLPKWGCLWGDT

SEQ ID NO: 262 - protein, 18 aa, Artificial Sequence
  sequence is synthesized
MDLADICLPKWGCLWESD

SEQ ID NO: 263 - protein, 18 aa, Artificial Sequence
  sequence is synthesized
MVLDDICLPRWGCLWSEK

SEQ ID NO: 264 - protein, 18 aa, Artificial Sequence
  sequence is synthesized
MWSGDLCLPRWGCLWGET

SEQ ID NO: 265 - protein, 18 aa, Artificial Sequence
  sequence is synthesized
NRMGDICLPRWGCLWDGH

SEQ ID NO: 266 - protein, 18 aa, Artificial Sequence
  sequence is synthesized
RDWEDLCLPNWGCLWELS

SEQ ID NO: 267 - protein, 18 aa, Artificial Sequence
  sequence is synthesized
RGDWDLCLPKWGCLWEGV

SEQ ID NO: 268 - protein, 18 aa, Artificial Sequence
  sequence is synthesized
RQWEDICLPRWGCLWGVG

SEQ ID NO: 269 - protein, 18 aa, Artificial Sequence
  sequence is synthesized
RVEYDLCLPRWGCLWEPP

SEQ ID NO: 270 - protein, 18 aa, Artificial Sequence
  sequence is synthesized
SIWSDICLPRWGCLWESD

SEQ ID NO: 271 - protein, 18 aa, Artificial Sequence
  sequence is synthesized
TDEWDICLPNWGCLWEAG

SEQ ID NO: 272 - protein, 18 aa, Artificial Sequence
  sequence is synthesized
TEDVDFCLPLWGCLWEEP

SEQ ID NO: 273 - protein, 18 aa, Artificial Sequence
  sequence is synthesized
VKEEDFCLPRWGCLWEAG

SEQ ID NO: 274 - protein, 18 aa, Artificial Sequence
  sequence is synthesized
WDFEDICLPRWGCLWADM

SEQ ID NO: 275 - protein, 18 aa, Artificial Sequence
  sequence is synthesized
WEDWDVCLPRWGCLWGGG

SEQ ID NO: 276 - protein, 18 aa, Artificial Sequence
  sequence is synthesized
YEDIDICLPRWGCLWDLS

SEQ ID NO: 277 - protein, 20 aa, Artificial Sequence
  sequence is synthesized
AGLDEDICLPRWGCLWGKEA

SEQ ID NO: 278 - protein, 20 aa, Artificial Sequence
  sequence is synthesized
AGMMGDICLPRWGCLWQGEP

SEQ ID NO: 279 - protein, 20 aa, Artificial Sequence
  sequence is synthesized
APGDWDFCLPKWGCLWDDDA

SEQ ID NO: 280 - protein, 20 aa, Artificial Sequence
  sequence is synthesized
AQLFDDICLPRWGCLWSDGY

SEQ ID NO: 281 - protein, 20 aa, Artificial Sequence
  sequence is synthesized
ARTMGDICLPRWGCLWGASD

SEQ ID NO: 282 - protein, 20 aa, Artificial Sequence
  sequence is synthesized
AWQDFDVCLPRWGCLWEPES

SEQ ID NO: 283 - protein, 20 aa, Artificial Sequence
  sequence is synthesized
DTTWGDICLPRWGCLWSEEA

SEQ ID NO: 284 - protein, 20 aa, Artificial Sequence
  sequence is synthesized
EGFLGDICLPRWGCLWGHQA

SEQ ID NO: 285 - protein, 20 aa, Artificial Sequence
  sequence is synthesized
EQWLHDICLPKWGCLWDDTD

SEQ ID NO: 286 - protein, 20 aa, Artificial Sequence
  sequence is synthesized
ETGWPDICLPRWGCLWEEGE

SEQ ID NO: 287 - protein, 20 aa, Artificial Sequence
  sequence is synthesized
FELGEDICLPRWGCLWEEHN

SEQ ID NO: 288 - protein, 20 aa, Artificial Sequence
  sequence is synthesized
GASLGDICLPRWGCLWGPED

SEQ ID NO: 289 - protein, 20 aa, Artificial Sequence
  sequence is synthesized
GEWWEDICLPRWGCLWGSSS

SEQ ID NO: 290 - protein, 20 aa, Artificial Sequence
  sequence is synthesized
GSLESDICLPRWGCLWGIDE

SEQ ID NO: 291 - protein, 20 aa, Artificial Sequence
  sequence is synthesized
GWLEEDICLPKWGCLWGADN

SEQ ID NO: 292 - protein, 20 aa, Artificial Sequence
  sequence is synthesized
HEQWDDICLPRWGCLWGGSY

SEQ ID NO: 293 - protein, 20 aa, Artificial Sequence
  sequence is synthesized
QRVDDDICLPRWGCLWGENS

SEQ ID NO: 294 - protein, 20 aa, Artificial Sequence
  sequence is synthesized
SVGWGDICLPKWGCLWAESD

SEQ ID NO: 295 - protein, 20 aa, Artificial Sequence
  sequence is synthesized
TLMSNDICLPRWGCLWDEPK

SEQ ID NO: 296 - protein, 20 aa, Artificial Sequence
  sequence is synthesized
TLVLDDICLPRWGCLWDMTD

SEQ ID NO: 297 - protein, 20 aa, Artificial Sequence
  sequence is synthesized
TWQGEDICLPRWGCLWDTEV

SEQ ID NO: 298 - protein, 20 aa, Artificial Sequence
  sequence is synthesized
VGVFDDICLPRWGCLWEQPV

SEQ ID NO: 299 - protein, 20 aa, Artificial Sequence
  sequence is synthesized
VPAMGDICLPRWGCLWEARN

SEQ ID NO: 300 - protein, 20 aa, Artificial Sequence
  sequence is synthesized
VSLGDDICLPKWGCLWEPEA

SEQ ID NO: 301 - protein, 20 aa, Artificial Sequence
  sequence is synthesized
VWIDRDICLPRWGCLWDTEN

SEQ ID NO: 302 - protein, 20 aa, Artificial Sequence
  sequence is synthesized
WRWNEDICLPRWGCLWEEEA

SEQ ID NO: 303 - protein, 20 aa, Artificial Sequence
  sequence is synthesized
AVSWADICLPRWGCLWERAD

SEQ ID NO: 304 - protein, 20 aa, Artificial Sequence
  sequence is synthesized
AWLDEDICLPKWGCLWNTGV

SEQ ID NO: 305 - protein, 20 aa, Artificial Sequence
  sequence is synthesized
FSLDEDICLPKWGCLWGAEK

SEQ ID NO: 306 - protein, 20 aa, Artificial Sequence
  sequence is synthesized
GDLGDDICLPRWGCLWDEYP

SEQ ID NO: 307 - protein, 20 aa, Artificial Sequence
  sequence is synthesized
GEGWSDICLPRWGCLWAEDE

SEQ ID NO: 308 - protein, 20 aa, Artificial Sequence
  sequence is synthesized
GLMGEDICLPRWGCLWKGDI

SEQ ID NO: 309 - protein, 20 aa, Artificial Sequence
  sequence is synthesized
GWHDRDICLPRWGCLWEQND

SEQ ID NO: 310 - protein, 20 aa, Artificial Sequence
  sequence is synthesized
LLGGHDICLPRWGCLWGGDV

SEQ ID NO: 311 - protein, 20 aa, Artificial Sequence
  sequence is synthesized
MRWSSDICLPKWGCLWGDEE

SEQ ID NO: 312 - protein, 20 aa, Artificial Sequence
  sequence is synthesized
QFEWDDICLPRWGCLWEVEV

SEQ ID NO: 313 - protein, 20 aa, Artificial Sequence
  sequence is synthesized
QGWWHDICLPRWGCLWEEGE

SEQ ID NO: 314 - protein, 20 aa, Artificial Sequence
  sequence is synthesized
REGWPDICLPRWGCLWSETG

SEQ ID NO: 315 - protein, 20 aa, Artificial Sequence
  sequence is synthesized
RELWGDICLPRWGCLWEHAT

SEQ ID NO: 316 - protein, 20 aa, Artificial Sequence
  sequence is synthesized
RLELMDICLPRWGCLWDPQD

SEQ ID NO: 317 - protein, 20 aa, Artificial Sequence
  sequence is synthesized
SGVLGDICLPRWGCLWEEAG

SEQ ID NO: 318 - protein, 20 aa, Artificial Sequence
  sequence is synthesized
SLGLTDLCLPRWGCLWEEEQ

SEQ ID NO: 319 - protein, 20 aa, Artificial Sequence
  sequence is synthesized
SSLEQDICLPRWGCLWGQDA

SEQ ID NO: 320 - protein, 20 aa, Artificial Sequence
  sequence is synthesized
SVLSDDICLPRWGCLWWDFS

SEQ ID NO: 321 - protein, 20 aa, Artificial Sequence
  sequence is synthesized
TSLLDDICLPRWGCLWYEEG

SEQ ID NO: 322 - protein, 20 aa, Artificial Sequence
  sequence is synthesized
TSLADDICLPRWGCLWSEDG

SEQ ID NO: 323 - protein, 20 aa, Artificial Sequence
  sequence is synthesized
VEMWHDICLPRWGCLWDSNA

SEQ ID NO: 324 - protein, 20 aa, Artificial Sequence
  sequence is synthesized
WDLASDICLPRWGCLWEEEA

SEQ ID NO: 325 - protein, 18 aa, Artificial Sequence
  sequence is synthesized
FITQDICLPRWGCLWGEN

SEQ ID NO: 326 - protein, 18 aa, Artificial Sequence
  sequence is synthesized
FLWRDICLPRWGCLWSEG

SEQ ID NO: 327 - protein, 18 aa, Artificial Sequence
  sequence is synthesized
FVHEDICLPRWGCLWGEG

SEQ ID NO: 328 - protein, 18 aa, Artificial Sequence
  sequence is synthesized
GLGDDICLPRWGCLWGRD

SEQ ID NO: 329 - protein, 18 aa, Artificial Sequence
  sequence is synthesized
GMFDDICLPKWGCLWGLG

SEQ ID NO: 330 - protein, 18 aa, Artificial Sequence
  sequence is synthesized
GPGWDICLPRWGCLWGEE

SEQ ID NO: 331 - protein, 18 aa, Artificial Sequence
  sequence is synthesized
GPWYDICLPRWGCLWDGV

SEQ ID NO: 332 - protein, 18 aa, Artificial Sequence
  sequence is synthesized
GWDDDICLPRWGCLWGDG

SEQ ID NO: 333 - protein, 18 aa, Artificial Sequence
  sequence is synthesized
LEYEDICLPKWGCLWGGE

SEQ ID NO: 334 - protein, 18 aa, Artificial Sequence
  sequence is synthesized
LLDEDICLPRWGCLWGVR

SEQ ID NO: 335 - protein, 18 aa, Artificial Sequence
  sequence is synthesized
LMSPDICLPKWGCLWEGD

SEQ ID NO: 336 - protein, 18 aa, Artificial Sequence
  sequence is synthesized
LVLGDICLPRWGCLWESD

SEQ ID NO: 337 - protein, 18 aa, Artificial Sequence
  sequence is synthesized
MLSRDICLPRWGCLWEEE

SEQ ID NO: 338 - protein, 18 aa, Artificial Sequence
  sequence is synthesized
MPWTDICLPRWGCLWSES

SEQ ID NO: 339 - protein, 18 aa, Artificial Sequence
  sequence is synthesized
RLGSDICLPRWGCLWGAG

SEQ ID NO: 340 - protein, 18 aa, Artificial Sequence
  sequence is synthesized
RLGSDICLPRWGCLWDYQ

SEQ ID NO: 341 - protein, 18 aa, Artificial Sequence
  sequence is synthesized
SPWMDICLPRWGCLWESG

SEQ ID NO: 342 - protein, 18 aa, Artificial Sequence
  sequence is synthesized
STFTDICLPRWGCLWELE

SEQ ID NO: 343 - protein, 18 aa, Artificial Sequence
  sequence is synthesized
SVLSDICLPRWGCLWEES

SEQ ID NO: 344 - protein, 18 aa, Artificial Sequence
  sequence is synthesized
TWFSDICLPRWGCLWEPG

SEQ ID NO: 345 - protein, 18 aa, Artificial Sequence
  sequence is synthesized
VHQADICLPRWGCLWGDT

SEQ ID NO: 346 - protein, 18 aa, Artificial Sequence
  sequence is synthesized
VLLGDICLPLWGCLWGED

SEQ ID NO: 347 - protein, 18 aa, Artificial Sequence
  sequence is synthesized
VNWGDICLPRWGCLWGES

SEQ ID NO: 348 - protein, 18 aa, Artificial Sequence
  sequence is synthesized
VVWSDICLPRWGCLWDKE

SEQ ID NO: 349 - protein, 18 aa, Artificial Sequence
  sequence is synthesized
VWYKDICLPRWGCLWEAE

SEQ ID NO: 350 - protein, 18 aa, Artificial Sequence
  sequence is synthesized
WDYGDICLPRWGCLWEEG

SEQ ID NO: 351 - protein, 18 aa, Artificial Sequence
  sequence is synthesized
WEVQDICLPRWGCLWGDD

SEQ ID NO: 352 - protein, 18 aa, Artificial Sequence
  sequence is synthesized
YIWRDICLPRWGCLWEGE

SEQ ID NO: 353 - protein, 18 aa, Artificial Sequence
  sequence is synthesized
YRDYDICLPRWGCLWDER

SEQ ID NO: 354 - protein, 18 aa, Artificial Sequence
  sequence is synthesized
AFWSDICLPRWGCLWEED

SEQ ID NO: 355 - protein, 18 aa, Artificial Sequence
  sequence is synthesized
DWGRDICLPRWGCLWDEE

SEQ ID NO: 356 - protein, 18 aa, Artificial Sequence
  sequence is synthesized
EAWGDICLPRWGCLWELE

SEQ ID NO: 357 - protein, 18 aa, Artificial Sequence
  sequence is synthesized
LILSDICLPRWGCLWDDT

SEQ ID NO: 358 - protein, 18 aa, Artificial Sequence
  sequence is synthesized
LKLEDICLPRWGCLWGES

SEQ ID NO: 359 - protein, 18 aa, Artificial Sequence
  sequence is synthesized
LLTRDICLPKWGCLWGSD

SEQ ID NO: 360 - protein, 18 aa, Artificial Sequence
  sequence is synthesized
LRWSDICLPRWGCLWEET

SEQ ID NO: 361 - protein, 18 aa, Artificial Sequence
  sequence is synthesized
LYLRDICLPKWGCLWEAD

SEQ ID NO: 362 - protein, 18 aa, Artificial Sequence
  sequence is synthesized
NWYDDICLPRWGCLWDVE

SEQ ID NO: 363 - protein, 17 aa, Artificial Sequence
  sequence is synthesized
QDWEDICLPRWGCLWGD

SEQ ID NO: 364 - protein, 18 aa, Artificial Sequence
  sequence is synthesized
QSWPDICLPKWGCLWGEG

SEQ ID NO: 365 - protein, 18 aa, Artificial Sequence
  sequence is synthesized
TLLQDICLPRWGCLWESD

SEQ ID NO: 366 - protein, 18 aa, Artificial Sequence
  sequence is synthesized
VRLMDICLPRWGCLWGEE

SEQ ID NO: 367 - protein, 18 aa, Artificial Sequence
  sequence is synthesized
VRWEDICLPRWGCLWGEE

SEQ ID NO: 368 - protein, 18 aa, Artificial Sequence
  sequence is synthesized
WDVADICLPRWGCLWAED

SEQ ID NO: 369 - protein, 18 aa, Artificial Sequence
  sequence is synthesized
WHMGDICLPRWGCLWSEV

SEQ ID NO: 370 - protein, 18 aa, Artificial Sequence
  sequence is synthesized
WKDFDICLPRWGCLWDDH

SEQ ID NO: 371 - protein, 18 aa, Artificial Sequence
  sequence is synthesized
WLSEDICLPQWGCLWEES

SEQ ID NO: 372 - protein, 18 aa, Artificial Sequence
  sequence is synthesized
WLSEDICLPRWGCLWAAD

SEQ ID NO: 373 - protein, 18 aa, Artificial Sequence
  sequence is synthesized
WLSDDICLPRWGCLWDDL

SEQ ID NO: 374 - protein, 20 aa, Artificial Sequence
  sequence is synthesized
EVREWDICLPRWGCLWENWR

SEQ ID NO: 375 - protein, 20 aa, Artificial Sequence
  sequence is synthesized
FGQEWDICLPRWGCLWGNEQ

SEQ ID NO: 376 - protein, 20 aa, Artificial Sequence
  sequence is synthesized
IWQLEDICLPRWGCLWEDGL

SEQ ID NO: 377 - protein, 20 aa, Artificial Sequence
  sequence is synthesized
NTPTYDICLPRWGCLWGDVP

SEQ ID NO: 378 - protein, 20 aa, Artificial Sequence
  sequence is synthesized
QPVWSDICLPRWGCLWGEDH

SEQ ID NO: 379 - protein, 19 aa, Artificial Sequence
  sequence is synthesized
SWYGGDICLPWGCLWSEES

SEQ ID NO: 380 - protein, 20 aa, Artificial Sequence
  sequence is synthesized
WGMARDWCLPMWGCLWRGGG

SEQ ID NO: 381 - protein, 20 aa, Artificial Sequence
  sequence is synthesized
WHLTDDICLPRWGCLWGDEQ

SEQ ID NO: 382 - protein, 20 aa, Artificial Sequence
  sequence is synthesized
NWAENDICLPRWGCLWGDEN

SEQ ID NO: 383 - protein, 20 aa, Artificial Sequence
  sequence is synthesized
SAREWDICLPTWGCLWEKDI

SEQ ID NO: 384 - protein, 18 aa, Artificial Sequence
  sequence is synthesized
AGEWDICLPRWGCLWDVE

SEQ ID NO: 385 - protein, 18 aa, Artificial Sequence
  sequence is synthesized
EIRWDFCLPRWGCLWDED

SEQ ID NO: 386 - protein, 18 aa, Artificial Sequence
  sequence is synthesized
ESLGDICLPRWGCLWGSG

SEQ ID NO: 387 - protein, 18 aa, Artificial Sequence
  sequence is synthesized
EYWGDICLPRWGCLWDWQ

SEQ ID NO: 388 - protein, 18 aa, Artificial Sequence
  sequence is synthesized
KMWSDICLPRWGCLWEEE

SEQ ID NO: 389 - protein, 18 aa, Artificial Sequence
  sequence is synthesized
MGTKDICLPRWGCLWAEA

SEQ ID NO: 390 - protein, 18 aa, Artificial Sequence
  sequence is synthesized
MHEWDICLPRWGCLWESS

SEQ ID NO: 391 - protein, 18 aa, Artificial Sequence
  sequence is synthesized
RGLHDACLPWWGCLWAGS

SEQ ID NO: 392 - protein, 18 aa, Artificial Sequence
  sequence is synthesized
RLFGDICLPRWGCLWQGE

SEQ ID NO: 393 - protein, 18 aa, Artificial Sequence
  sequence is synthesized
SGEWDICLPRWGCLWGEG

SEQ ID NO: 394 - protein, 18 aa, Artificial Sequence
  sequence is synthesized
SMFFDHCLPMWGCLWAEQ

SEQ ID NO: 395 - protein, 18 aa, Artificial Sequence
  sequence is synthesized
VGEWDICLPNWGCLWERE

SEQ ID NO: 396 - protein, 18 aa, Artificial Sequence
  sequence is synthesized
WWMADRCLPLWGCLWRGD

SEQ ID NO: 397 - protein, 18 aa, Artificial Sequence
  sequence is synthesized
WWVRDLCLPTWGCLWSGK

SEQ ID NO: 398 - protein, 18 aa, Artificial Sequence
  sequence is synthesized
YFDGDICLPRWGCLWGSD

SEQ ID NO: 399 - protein, 18 aa, Artificial Sequence
  sequence is synthesized
TLFQDICLPRWGCLWEES

SEQ ID NO: 400 - protein, 18 aa, Artificial Sequence
  sequence is synthesized
WFPKDRCLPVWGCLWERH

SEQ ID NO: 401 - protein, 20 aa, Artificial Sequence
  sequence is synthesized
QRLMEDICLPRWGCLWEDDF

SEQ ID NO: 402 - protein, 18 aa, Artificial Sequence
  sequence is synthesized
RLIEDICLPRWGCLWEDD

SEQ ID NO: 403 - protein, 17 aa, Artificial Sequence
  sequence is synthesized
QRLMEDICLPRWGCLWE

SEQ ID NO: 404 - protein, 20 aa, Artificial Sequence
  sequence is synthesized
GEWWEDICLPRWGCLWEEED

SEQ ID NO: 405 - protein, 20 aa, Artificial Sequence
  sequence is synthesized
QRLIEDICLPRWGCLWEDDF

SEQ ID NO: 406 - protein, 17 aa, Artificial Sequence
  sequence is synthesized
RLIEDICLPRWGCLWED

SEQ ID NO: 407 - protein, 16 aa, Artificial Sequence
  sequence is synthesized
RLIEDICLPRWGCLWE

SEQ ID NO: 408 - protein, 15 aa, Artificial Sequence
  sequence is synthesized
RLIEDICLPRWGCLW

SEQ ID NO: 409 - protein, 14 aa, Artificial Sequence
  sequence is synthesized
RLIEDICLPRWGCL

SEQ ID NO: 410 - protein, 13 aa, Artificial Sequence
  sequence is synthesized
RLIEDICLPRWGC

SEQ ID NO: 411 - protein, 16 aa, Artificial Sequence
  sequence is synthesized
LIEDICLPRWGCLWED

SEQ ID NO: 412 - protein, 15 aa, Artificial Sequence
  sequence is synthesized
IEDICLPRWGCLWED

SEQ ID NO: 413 - protein, 14 aa, Artificial Sequence
  sequence is synthesized
EDICLPRWGCLWED

SEQ ID NO: 414 - protein, 13 aa, Artificial Sequence
  sequence is synthesized
DICLPRWGCLWED

SEQ ID NO: 415 - protein, 12 aa, Artificial Sequence
  sequence is synthesized
ICLPRWGCLWED

SEQ ID NO: 416 - protein, 11 aa, Artificial Sequence
  sequence is synthesized
CLPRWGCLWED

SEQ ID NO: 417 - protein, 14 aa, Artificial Sequence
  sequence is synthesized
IEDICLPRWGCLWE

SEQ ID NO: 418 - protein, 12 aa, Artificial Sequence
  sequence is synthesized
EDICLPRWGCLW

SEQ ID NO: 419 - protein, 10 aa, Artificial Sequence
  sequence is synthesized
DICLPRWGCL

SEQ ID NO: 420 - protein, 10 aa, Artificial Sequence
  sequence is synthesized
ICLPRWGCLW

SEQ ID NO: 421 - protein, 8 aa, Artificial Sequence
  sequence is synthesized
ICLPRWGC

SEQ ID NO: 422 - protein, 4 aa, Artificial Sequence
  sequence is synthesized
GGGS

SEQ ID NO: 423 - protein, 11 aa, Artificial Sequence
  sequence is synthesized
DXCLPXWGCLW

SEQ ID NO: 424 - protein, 13 aa, Artificial Sequence
  sequence is synthesized
XDICLPRWGCLWX

SEQ ID NO: 425 - protein, 13 aa, Artificial Sequence
  sequence is synthesized
XDICLPRWGCLWX

SEQ ID NO: 426 - protein, 15 aa, Artificial Sequence
  sequence is synthesized
XXEMCYFPGICWMXX

SEQ ID NO: 427 - protein, 15 aa, Artificial Sequence
  sequence is synthesized
XXDLCLRDWGCLWXX

SEQ ID NO: 428 - protein, 107 aa, Artificial sequence [sequence omitted]

SEQ ID NO: 429 - protein, 120 aa, Artificial sequence [sequence omitted]

(19) United States
(12) Patent Application Publication
(10) Pub. No.: US 2006/0073152 A1
(43) Pub. Date: Apr. 6, 2006

(54) Therapeutic agents with decreased toxicity

(75) Inventor: Mark S. Dennis, San Carlos, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/233,256

(22) Filed: Sep. 22, 2005

Related U.S. Application Data
(60) Provisional application No. 60/641,534, filed on Jan. 5, 2005.
(60) Provisional application No. 60/616,507, filed on Oct. 5, 2004.

(51) Int. Cl. A61K 39/395; C07K 16/46; C07H 21/04; C12P 21/04

(52) U.S. Cl. 424/155.1; 424/178.1; 530/391.1; 435/69.7; 435/320.1; 435/328; 536/23.53

(57) ABSTRACT

The present invention relates to therapeutic agents with reduced toxicity comprising a serum albumin binding peptide (SABP), a targeting agent and a cytotoxic agent. The present invention also relates to methods for reducing the toxicity of therapeutic agents and methods of treatment using the therapeutic agents with reduced toxicity.

This application is a non-provisional application filed under 37 CFR 1.53(b)(1), claiming priority under 35 USC 119(e) to provisional application No. 60/641,534 filed on Jan. 5, 2005 and 60/616,507 filed on Oct. 5, 2004, the contents of which are incorporated herein by reference.

### FIELD OF THE INVENTION

This invention relates to novel therapeutic agents with decreased toxicity in vivo, compositions comprising the same, methods for decreasing the toxicity of therapeutic agents in vivo and methods for treating patients comprising administering the novel therapeutic agents.

### BACKGROUND OF THE INVENTION

Attempts have been made to use antibody-drug conjugates (ADC), to locally deliver cytotoxic or cytostatic agents, i.e. drugs that kill or inhibit tumor cells in the treatment of cancer (Payne, G. (2003) Cancer Cell 3:207-212; Syrigos and Epenetos (1999) Anticancer Research 19:605-614; Niculescu-Duvaz and Springer (1997) Adv. Drug Del. Rev. 26:151-172; U.S. Pat. No. 4,975,278). Theoretically, the drug moiety will be targeted to the tumors and be internalized, wherein systemic administration of these unconjugated drug agents may result in unacceptable levels of toxicity to normal cells (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies '84: Biological And Clinical Applications, A. Pinchera et al. (ed.s), pp. 475-506).

Both polyclonal antibodies and monoclonal antibodies have been used in to make ADCs (Rowland et al., (1986) Cancer Immunol. Immunother., 21:183-87). Drugs used in these methods include daunomycin, doxorubicin, methotrexate and vindesine (Rowland et al., (1986) supra). Toxins used in antibody-toxin conjugates include bacterial toxins such as diphtheria toxin, plant toxins such as ricin, small molecule toxins such as geldanamycin (Mandler et al (2000) J. of the Nat. Cancer Inst. 92(19):1573-1581; Mandler et al (2000) Bioorganic & Med. Chem. Letters 10:1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342). More recently, auristatin peptides, auristatin E (AE) and monomethylauristatin (MMAE) and synthetic analogs of dolastatin (WO 02/088172), have been conjugated to full length antibodies (e.g., Klussman, et al (2004), Bioconjugate Chemistry 15(4):765-773; Doronina et al (2003) Nature Biotechnology 21(7):778-784; Francisco et al (2003) Blood 102(4):1458-1465; US 2004/0018194; WO 04/032828; Mao, et al (2004) Cancer Res. 64(3):781-788; Bhaskar et al (2003) Cancer Res. 63:6387-6394; WO 03/043583; Mao et al (2004) Cancer Res. 64:781-788). Variants of auristatin E are also disclosed in U.S. Pat. No. 5,767,237; U.S. Pat. No. 6124431.

ZEVALIN® (ibritumomab tiuxetan, Biogen Idec Inc.) is an antibody-radioisotope conjugate composed of a murine IgG1 kappa monoclonal antibody directed against the CD20 antigen found on the surface of normal and malignant B lymphocytes and ¹¹¹In or ⁹⁰Y radioisotope bound by a thiourea linker-chelator (Wiseman et al (2000) Eur. J. Nucl. Med. 27(7):766-77; Wiseman et al (2002) Blood 99(12): 4336-42; Witzig et al (2002) J. Clin. Oncol. 20(10):2453-63; Witzig et al (2002) J. Clin. Oncol. 20(15):3262-69). Although ZEVALIN® has activity against B-cell non-Hodgkin's Lymphoma (NHL), administration results in severe and prolonged cytopenias in most patients. MYLOTARG™ (gemtuzumab ozogamicin, Wyeth Pharmaceuticals), an antibody drug conjugate composed of a CD33 antibody linked to calicheamicin, was approved in 2000 for the treatment of acute myeloid leukemia by injection (Drugs of the Future (2000) 25(7):686; U.S. Pat. Nos. 4,970,198; 5,079,233; 5,585,089; 5,606,040; 5,693,762; 5,739,116; 5,767,285; 5,773,001). Cantuzumab mertansine (Immunogen, Inc.), an antibody drug conjugate composed of the huC242 antibody linked via the disulfide linker SPP to the maytansinoid drug moiety, DM1 (Xie et al (2004) J. of Pharm. and Exp. Ther. 308(3):1073-1082), is advancing into Phase II trials for the treatment of cancers that express CanAg, such as colon, pancreatic, gastric, and others. MLN-2704 (Millennium Pharm., BZL Biologics, Immunogen Inc.), an antibody drug conjugate composed of the anti-prostate specific membrane antigen (PSMA) monoclonal antibody linked to the maytansinoid drug moiety, DM 1, is under development for the potential treatment of prostate tumors.

Various methods have been tried to improve the half life of small molecule or biological therapeutics. For example, glycosylation sites have been introduced to the molecules (Keyt et. al., 1994, PNAS USA 91:3670-74), and molecules have been conjugated with PEG (Clark et.al., 1996, J. Biol. Chem., 271: 21969-77; Lee et. al, 1999, Bioconjugate Chem. 10:973-981; Tanaka et. al., 1991, Cancer Res. 51:3710-14) to increase size and increase elimination half-times. Some have attempted to use human serum albumin to improve the therapeutic use of the drug. For example, albumin has been attached to small molecules (Syed et. al., 1997, Blood 89:3243-3252; Burger et. al., 2001 Int. J. Cancer 92:718-724; Wosikowski K, et al., Clin Cancer Res. 2003 May 9(5):1917-26); CD4 (Yeh et. al., 1992, PNAS USA 89:1904-1908); the Fc portion of an IgG (Ashkenazi et. al. (1997) Curr. Opin in Immunol. 9:195-200), IL-2 (Yao, Z et al., (2004 May) Cancer Immunol Immunother. 53(5):404-10) and the bridge between an anti-gp72 antibody and a methotrexate molecule (Affleck, K et al., (1992) Br J Cancer.65(6):838-44).

The use of albumin binding polypeptides have also been investigated. Extended in vivo half-times of human soluble complement receptor type 1 (sCR1) fused to the albumin binding domains from Streptococcal protein G have been reported (Makrides et al. 1996 J. Phannacol. Exptl. Ther. 277:532-541). Labelled albumin binding domains of protein G have been described (EP 0 486,525). Several phage diplay-derived albumin binding peptides have been described by applicant. See WO 01/45746, United States Patent Publication No. 2004/0001827, and Dennis, M S, et al., (2002) JBC 277(38):35035-43. In theory, serum albumin binding peptides associate with serum albumin non-covalently in vivo. As such, the serum albumin binding peptides are necessarily a step removed from the in vivo cycling mechanism of serum albumin itself.

The invention described below addresses the unexpectedly advantageous utility of albumin binding peptides in the context of a conjugate with a targeting agent/cytoxic agent.

### SUMMARY OF THE INVENTION

The present invention relates to a conjugate molecule comprising a covalently linked combination of at least one serum albumin-binding moiety (SABM), targeting agent (TA) and cytotoxic agent (CA). According to one embodiment, the conjugate molecule comprises 2 or more CAs. According to embodiment, the conjugate molecule comprises 2 or more TAs.

According to one embodiment of this invention, the SABM comprises an amino acid sequence that is at least 50% identical to the sequence of DICLPRWGCLW (SEQ ID NO:8) and wherein the amino acid sequence has two Cys residues with five amino acid residues in between the Cys residues. According to one embodiment, the amino acid sequence has a percent identity to SEQ ID NO:8 that is selected from the group consisting of at least 60% identity, at least 70% identity, at least 80% identity, at least 85% identity, at least 90% identity, at least 95% identity, at least 98% identity and at least 99% identity.

According to another embodiment, the SABM comprises a variant of the amino acid sequence of DICLPRWGCLW (SEQ ID NO:8), wherein between 1-5 residues of any of one of the residues of SEQ ID NO:8 is substituted with a different amino acid residue, except for the Cys residues.

According to another embodiment, the SABM comprises a linear or a cyclic amino acid sequence selected from the group consisting of:

| | |
|---|---|
| Xaa-Xaa-Cys-Xaa-Xaa-Xaa-Xaa-Xaa- | [SEQ ID NO: 1] |
| Cys-Xaa-Xaa-Phe-Cys-Xaa-Asp-Trp- | |
| Pro-Xaa-Xaa-Xaa-Ser-Cys | |
| Val-Cys-Tyr-Xaa-Xaa-Xaa-Ile-Cys-Phe | [SEQ ID NO: 2] |
| Cys-Tyr-Xaa1-Pro-GIy-Xaa-Cys | [SEQ ID NO: 3] |
| Asp-Xaa-Cys-Leu-Pro-Xaa-Trp-Gly- | [SEQ ID NO: 4] |
| Cys-Leu-Trp | |
| Trp-Cys-Asp-Xaa-Xaa-Leu-Xaa-Ala-; | [SEQ ID NO: 5] |
| Xaa-Asp-Leu-Cys | |
| Asp-Leu-Val-Xaa-Leu-Gly-Leu-Glu-; | [SEQ ID NO: 6] |
| Cys-Trp | |
| CXXGPXXXXC | [SEQ ID NO:21] |
| XXXXCXXGPXXXXCXXXX | [SEQ ID NO:22] |
| CXXXXXXCXXXXXXCCXXXCXXXXXXC | [SEQ ID NO:23] |
| CCXXXCXXXXXXC | [SEQ ID NO:24] |
| CCXXXXXCXXXXCXXXXCC | [SEQ ID NO:25] |
| CXCXXXXXXXCXXXCXXXXXX | [SEQ ID NO:26] |
| XXXXXDXCLPXWGCLWXXXX | [SEQ ID NO:155] |
| XXXXDXCLPXWGCLWXXX | [SEQ ID NO:156] |
| D X C L P X W G C L W | [SEQ ID NO:423] |
| X X X X D I C L P R W G C L W X , | [SEQ ID NO:424] |
| X X, | |
| X X X X X D I C L P R W G C L W X | [SEQ ID NO:425] |
| X X X | |
| X X E M C Y F P G I C W M X X | [SEQ ID NO:426] |
| X X D L C L R D W G C L W X X | [SEQ ID NO:427] |

wherein X is any amino acid residue.

According to one preferred embodiment, SABM sequence of the above general formulae, particularly SEQ ID NO: 1, SEQ ID NO: 2, SEQ ID NO: 3and SEQ ID NO: 4, comprise additional amino acids at the N-terminus (Xaa)ₓ and additional amino acids at the C-terminus (Xaa)_{z}, wherein Xaa is an amino acid and x and z are a whole number greater or equal to 0 (zero), generally less than 100, preferably less than 10 and more preferably 0, 1, 2, 3, 4 or 5 and more preferably 4 or 5 and Xaa₁ is selected from the group consisting of Ile, Phe, Tyr, and Val. In one embodiment, the invention relates to the use of an albumin binding peptide comprising the sequence DICLPRWGCLW [SEQ ID NO: 8]. According to one embodiment, the SABM comprises any one of the amino acid sequences selected from the group consisting of SEQ ID NOs: 7-20,27-154 and 157-421. According one preferred embodiment, the SABM comprises the amino acid sequence selected from the group consisting of: SEQ ID NOs: 7, 8, 9, 10, 11, 12, 13, 14, 15, 16, 17, 18, 19 and 20.

According to another embodiment, the SABM comprises the following amino acid sequence:
Xaaᵢ-Cys-Xaaⱼ-Cys-Xaaₖ, wherein the sum of i, j, and k is about 25 or less and Xaa is any amino acid residue. According to one preferred embodiment, the sum of i, j, and k is about 18 residues or less. According to another preferred embodiment, the the sum of i, j, and k is about 11 residues or less.

According to another embodiment, the SABM comprises any one of the peptide sequences described in Tables 1-9.

According to one embodiment of this invention, all the above-mentioned SABM sequences bind to serum albumin with a K_{d} that is about 100 μM or less. According to another embodiment, the K_{d} is selected from the group consisting of about 10 μM or less, about 1 μM or less, about 500 nM or less, about 100 nM or less, about 50 nM or less and about 10 nM or less.

According to another embodiment, the TA is a polypeptide comprising an amino acid sequence that can bind to a target cell surface protein, wherein the TA comprises an amino acid sequence that is a ligand for the cell surface protein, an adhesion or an antibody, or a fragment of any one of the above that can bind to the cell surface protein. According to one embodiment, the cell surface protein to be targeted is a B cell surface marker. According to another embodiment, the receptor to be targeted is selected from the group consisting of HER2, CD20, EGFR, PDGFR, BR3, Flt-1, KDR and EphB2. According to another embodiment, the TA is an antibody directed against any one of those receptors. According to a preferred embodiment, the antibody is in the form of any one of the following: a Fab, F(ab)₂, scFv and a diabody. According to another embodiment, the TA comprises a VH or VL sequence described herein (e.g., an anti-HER2 antibody comprising the antigen-binding portions of SEQ ID NO:428 and 429).

According to one embodiment, the anti-HER2 antibody comprises the variable regions of SEQ ID NO:428 and 429. According to one embodiment, the anti-HER2 antibody comprises a variant of the light chain variable sequence of SEQ ID NO:428, wherein at least one or more of the amino acids selected from the group consisting of Q27(V_{L}); D28(V_{L}), N30(V_{L}), T31(V_{L}), A32(V_{L}), Y49(V_{L}), F53(V_{L}), Y55(V_{L}), R66(V_{L}), H91(V_{L}), Y92((V_{L}), and T94(V_{L}), numbered according to the Kabat numbering system, are substituted with any amino acid other than alanine. According to one embodiment, the anti-HER2 antibody comprises a variant of the light chain variable sequence of SEQ ID NO:428, wherein at least one or more amino acids of the variable region have a substitution selected from the group consisting of D28(V_{L})Q; D28(V_{L})G; N30(V_{L})S; T31(V_{L})S; A32(V_{L})G; Y49(V_{L})W, Y49(V_{L})D, Y49(V_{L})V; F53(V_{L})W; F53(V_{L})V, F53(V_{L})Q, Y55(V_{L})W, R66(V_{L})N, H91(V_{L})F, H91(V_{L})Y, Y92(V_{L})W, and T94(V_{L})S. According to one embodiment, the anti-HER2 antibody comprises a variant of the light chain variable sequence of SEQ ID NO:428, wherein the variable region comprises at least three substitutions Y49(V_{L})D, F53(V_{L})W, and Y55(V_{L})W. According to one embodiment, the anti-HER2 antibody comprises a variant of the light chain variable sequence of SEQ ID NO:428, wherein the variable region comprises at least three substitutions N30(V_{L})S, H91(V_{L})F, and Y92(V_{L})W.

According to one embodiment, the anti-HER2 antibody comprises a variant of the heavy chain variable sequence of SEQ ID NO:429, wherein at least one or more of the amino acids selected from the group consisting of W95(V_{H}), D98(V_{H}), F100(V_{H}), Y100a(V_{H}), and Y102(V_{H}), numbered according to the Kabat numbering system, are substituted with any amino acid other than alanine. According to one embodiment, the anti-HER2 antibody comprises a variant of the heavy chain variable sequence of SEQ ID NO:429, wherein the variable region comprises at least one or more substitutions selected from the group consisting of W95(V_{H})Y, D98(V_{H})W, D98(V_{H})R, D98(V_{H})K, D98(V_{H})H, F100(V_{H})P, F100(V_{H})L, F100(V_{H})M, F100(V_{H})W, Y100a(V_{H})F, Y102(V_{H})V, Y102(V_{H})K, and Y102(V_{H})L. According to one embodiment, the anti-HER2 antibody comprises a variant of the heavy chain variable sequence of SEQ ID NO:429, wherein the variable region comprises at least the substitutions F100(V_{H})P and Y102(V_{H})K. According to one embodiment, the anti-HER2 antibody comprises a variant of the heavy chain variable sequence of SEQ ID NO:429, wherein the variable region comprises at least the substitutions of F100(V_{H})P and Y102(V_{H})L.

According to one embodiment, the anti-HER2 antibody comprises variants of the light chain variable sequence SEQ ID NO:428 and heavy chain variable sequence SEQ ID NO:429, wherein at least one or more of the amino acids selected from the group consisting of D28(V_{L}), N30(V_{L}), T31(V_{L}), A32(V_{L}), Y49(V_{L}), F53(V_{L}), Y55(V_{L}), R66(V_{L}), H91(V_{L}), Y92(V_{L}), T94(V_{L}), W95(V_{H}), D98(V_{H}), F100(V_{H}); Y100a(V_{H}), and Y102(V_{H}), numbered according to the Kabat numbering system, are substituted with any amino acid other than alanine. According to one embodiment, the anti-HER2 antibody comprises variants of the light chain variable sequence SEQ ID NO:428 and heavy chain variable sequence SEQ ID NO:429 comprising at least one or more of the following substitutions D28(V_{L})Q; D28(V_{L})G; N30(V_{L})S; T31(V_{L})S; A32(V_{L})G; Y49(V_{L})W, Y49(V_{L})D, Y49(V_{L})V; F53(V_{L})W, F53(V_{L})V, F53(V_{L})Q, Y55(V_{L})W, R66(V_{L})N, H91(V_{L})F, H91(V_{L})Y, Y92(V_{L})W, T94(V_{L})S, W95(V_{H})Y, D98(V_{H})W, D98(V_{H})R, D98(V_{H})K, D98(V_{H})H, F100(V_{H})P, F100(V_{H})L, F100(V_{H})M, Y100a(V_{H})F, Y102(V_{H})V, Y102(V_{H})K, and Y102(V_{H})L. According to one embodiment, the anti-HER2 antibody comprises variants of the light chain variable sequence SEQ ID NO:428 and heavy chain variable sequence SEQ ID NO:429 comprising at least the following substitutions Y49(V_{L})D, F53(V_{L})W, Y55(V_{L})W, F100(V_{H})P, and Y102(V_{H})K. According to one embodiment, the anti-HER2 antibody comprises variants of the light chain variable sequence SEQ ID NO:428 and heavy chain variable sequence SEQ ID NO:429 comprising at least the following substitutions Y49(V_{L})D, F53(V_{L})W, Y55(V_{L})W, F100(V_{H})P, and Y102(V_{H})L. According to another embodiment, the anti-HER2 antibody is any anti-HER2 antibody disclosed in United States Patent Publication No. 2003/0228663 A1, filed Apr. 9, 2003; WO 03/087131; Carter et al., (1992) PNAS 89:4285-4289 which publications are expressly incorporated by reference herein.

According to one embodiment, the TA has an additional bioactivity other than the ability to bind to a protein on the outer surface of a cell. According to another embodiment, the other bioactivity is the ability to block ligand-mediated cellular signaling through the cell. According to another embodiment, the other bioactivity is the ability to induce apoptosis of the targeted cell. According to another embodiment, the TA is a polypeptide that binds to a protein on a cell of interest with a Kd selected from the group consisting of 10 uM or less, 1 uM or less, 500 nm or less, 100 nm or less and 10 nm or less.

According to one embodiment, the protein on the cell of interest to which the TA binds is overexpressed in cancer cells as compared to normal cells. According to another embodiment, the cell being targeted by the TA is a pathogenic cell, such as a tumor cell.

According to one preferred embodiment, the cytotoxic agent is monomethylauristatin (MMAE).

According to another preferred embodiment, the conjugate molecule comprises a linker moiety located between said SABM and targeting agent or cytotoxic agent. In one embodiment, the linker moiety comprises the amino acid sequence: GGGS (SEQ ID NO:422).

According to another embodiment the SABM binds to human albumin. According to another embodiment, the SABM is conjugated to the N- or C-terminal region of a variable heavy or variable light chain of a TA.

The present invention provides compositions comprising the conjugate molecule admixed with a pharmaceutical carrier. The present invention also provides a the use of the conjugate molecule in the manufacture of a medicament.

The present invention also provides methods for reducing the toxicity of a therapeutic agent comprising the step of producing a therapeutic agent with a serum albumin binding moiety (SABM) conjugated to the therapeutic agent. The method can further comprise the step of comparing the toxicity of the therapeutic agent having the SABM with the therapeutic agent without the SABM. According to one embodiment, the method further comprises the step of measuring the toxicity of the therapeutic agent:SABM conjugate.

The present invention provides methods of reducing the toxicity of a therapeutic agent in a mammal comprising administering to the mammal a therapeutically effective amount of the conjugate molecule according to this invention. According to one embodiment, the method further comprises the step of measuring the toxicity of the therapeutic agent:SABM conjugate. According to one preferred embodiment, the mammal is suffering from an autoimmune disease or a cancer.

The present invention provides methods of treating a tumor in a mammal comprising the step of treating a mammal having the tumor with a therapeutically effective amount of a conjugate molecule of this invention that binds to the tumor cells or vasculature surrounding the tumor. The present invention also provides methods of treating an autoimmune disorder in a mammal comprising the step of treating a mammal having the autoimmune disorder with a therapeutically effective amount of a conjugate molecule of this invention. According to one preferred embodiment, the conjugate molecules bind to B-cells that contribute to or cause the autoimmune disorder. The present invention also provides methods of treating a cell proliferative disorder in a mammal comprising the step of treating a mammal having the autoimmune disorder with a therapeutically effective amount of a conjugate molecule of this invention. According to another embodiment, the present invention provides a method for depleting B cells in a mammal comprising the step of treating the mammal with a therapeutically effective amount of a conjugate molecule of this invention that binds to the B cell.

According to one embodiment, the methods of treatment of this invention further comprises the step of measuring the toxicity of the conjugate molecule in a mammal.

According to one embodiment, toxicity is manifested as any one of the group consisting of weight loss, hematopoietic toxicity, renal toxicity, liver toxicity, gastrointestinal toxicity, decreased mobilization of hematopoietic progenitor cells from bone marrow into the peripheral blood, anemia, myelosuppression, pancytopenia, thrombocytopenia, neutropenia, lymphopenia, leukopenia, stomatitis, alopecia, headache, and muscle pain.

The present invention also provides articles of manufacture comprising a container, a composition within the container comprising a conjugate molecule of this invention, a package insert containing instructions to administer a therapeutically effective dose.

### BRIEF DESCRIPTION OF THE DRAWINGS

FIG. 1 shows the tumor volume over time post injection of a control vehicle (circles), Herceptin®-vc-Pab-MMAE (squares), Ab.Fab4D5-H-vc-PAB-MMAE (diamonds), Fab3D4-vc-PAB-MMAE (triangles) and Ab.FabControl-vc-PAB-MMAE (empty circles).

FIG. 2 shows the group change in body weight post administration of Herceptin®-vc-MMAE (squares), Herceptin®-F(ab′)₂4D5-vc-MMAE (crosses), free MMAE (circles).

FIG. 3 shows the group change in body weight post administration of Herceptin®-vc-MMAE (diamonds), Fab4D5-vc-MMAE (triangles), AB.Fab4D5-H-vc-MMAE (circles) and PBS (squares).

FIG. 4 shows the amino acid sequence of a light chain variable domain of a humanized anti-HER2 antibody [SEQ ID NO:428] and a heavy chain variable domain of a humanized anti-HER2 antibody [SEQ ID NO:429].

### DETAILED DESCRIPTION OF THE PREFERRED EMBODIMENTS

### I. DEFINITIONS

The term “serum albumin binding peptide” or “serum albumin binding moiety” (“SABM”) refers to a compound or a polypeptide comprising an amino acid sequence that binds to serum albumin. According to one preferred embodiment, the SABM binds to a human serum albumin. According to one embodiment, the SABM comprises at least one of any one of the sequences recited in the Listing of Sequences that binds to rabbit, rat, mouse or human serum albumin. According to another embodiment, the SABM comprises at least one of any one of the sequences recited in the Listing of Sequences that binds to multiple species of serum albumin. According to one embodiment, the SABM comprises at least one of any one of the sequences recited in Tables 1-9 that binds to any one or combination of rabbit, rat, mouse and human serum albumin. According to another embodiment, the SABM comprises at least one of any one of the sequences recited in the Tables 1-9 that binds to multiple species of serum albumin. Examples of multispecies binders include those SABM's that bind at least human and rat serum albumin; those that bind at least human, rat and rabbit serum albumin; those that bind at least human and rabbit serum albumin; and those that bind at least human and mouse serum albumin.

According to one preferred embodiment, the SABM peptide is a non-naturally occurring amino acid sequence that can bind albumin. SABMs within the context of the present invention can be constrained (that is, having some element of structure as, for example, the presence of amino acids which initiate a beta-turn or beta- pleated sheet, or for example, cyclized by the presence of disulfide-bonded Cys residues) or unconstrained (linear) amino acid sequences of less than about 50 amino acid residues, and preferably less than about 40 amino acids residues. Of the SABMs less than about 40 amino acid residues, preferred are the SABMs of between about 10 and about 30 amino acid residues and especially the SABMs of about 20 amino acid residues. However, upon reading the instant disclosure, the skilled artisan will recognize that it is not the length of a particular SABM but its ability to bind an albumin that distinguishes the SABM of the present invention.

A “targeting agent” or “TA” of the present invention will bind a target molecule on the surface of a cell with sufficient affinity and specificity if the TA “homes” to, “binds” or “targets” a target molecule such as a specific cell type bearing the target molecule in vitro and preferably in vivo (see, for example, the use of the term “homes to,” “homing,” and “targets” in Pasqualini and Ruoslahti, 1996 Nature, 380:364-366 and Arap et al., 1998 Science, 279:377-380). In general, the TA will bind a target molecule with an affinity characterized by a dissociation constant, K_{d}, of less than about 10 microM, preferably less than about 100 nM and less than about 10 nM. However, polypeptides or small molecules having an affinity for a target molecule of less than about 1 nM and preferably between about 1 pM and 1 nM are equally likely to be TAs within the context of the present invention. Preferably, the TA is a polypeptide (e.g., an antibody). In general, a TA that binds a particular target molecule as described above can be isolated and identified by any of a number of techniques known in the art.

TAs are amino acid sequences as described above that may contain naturally as well as non-naturally occurring amino acid residues, such as phage-display derived antibodies. So-called “peptide mimetics” and “peptide analogs”, that include non-amino acid chemical structures that mimic the structure of a particular amino acid or peptide, can be TAs within the context of the invention. Such mimetics or analogs are characterized generally as exhibiting similar physical characteristics such as size, charge or hydrophobicity present in the appropriate spatial orientation as found in their peptide counterparts. A specific example of a peptide mimetic compound is a compound in which the amide bond between one or more of the amino acids is replaced by, for example, a carbon-carbon bond or other bond as is well known in the art (see, for example Sawyer, 1995, In: Peptide Based Drug Design pp. 378-422, ACS, Wash. DC).

A “B cell surface marker” or “B cell surface antigen” herein is an antigen expressed on the surface of a B cell which can be targeted with an antagonist which binds thereto. Exemplary B cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers (for descriptions, see The Leukocyte Antigen Facts Book, 2nd Edition. 1997, ed. Barclay et al. Academic Press, Harcourt Brace & Co., New York). Other B cell surface markers include RP105, FcRH2, CD79A, C79B, CR2, CCR6, CD72, P2X5, HLA-DOB, CXCR5, FCER2, BR3, BTLA, NAG14 (aka LRRC4), SLGC16270 (ala LOC283663), FcRH1, IRTA2, ATWD578 (aka MGC15619), FcRH3, IRTA1, FcRH6 (aka LOC343413) and BCMA (aka TNFRSF17).

The B cell surface marker of particular interest is preferentially expressed on B cells compared to other non-B cell tissues of a mammal and may be expressed on both precursor B cells and mature B cells. The preferred B cell surface markers herein are CD20 and CD22.

The “CD20” antigen is non-glycosylated phosphoprotein found on the surface of greater than 90% of B cells from peripheral blood or lymphoid organs. CD20 is expressed during early pre-B cell development and remains until plasma cell differentiation. CD20 is present on both normal B cells as well as malignant B cells. Other names for CD20 in the literature include “B-lymphocyte-restricted antigen” B1 and “Bp35”. The CD20 antigen is described in Clark et al. PNAS (USA) 82:1766 (1985), for example. The amino acid sequence of human CD20 is shown in The Leukocyte Antigen Facts Book, Barclay et al. supra, page 182, and also EMBL Genbank accession no. X12530 and Swissprot P11836.

The “CD22” antigen, also known as BL-CAM or Lyb8, is a type 1 integral membrane glycoprotein with molecular weight of about 130 (reduced) to 140 kD (unreduced). It is expressed in both the cytoplasm and cell membrane of B-lymphocytes. CD22 antigen appears early in B-cell lymphocyte differentiation at approximately the same stage as the CD19 antigen. Unlike other B-cell markers, CD22 membrane expression is limited to the late differentiation stages comprised between mature B cells (CD22+) and plasma cells (CD22−). The CD22 antigen is described, for example, in Wilson et al. J. Exp. Med. 173:137 (1991) and Wilson et al. J. Immunol. 150:5013 (1993).

The “CD19” antigen refers to an antigen identified, for example, by the HD237-CD19 or B4 antibody (Kiesel et al. Leukemia Research II, 12: 1119 (1987)). CD19 is found on Pro-B, pre-B, immature and mature, activated and memory B cells, up to a point just prior to terminal differentiation into plasma cells. Neither CD19 nor CD20 is expressed on hematopoietic stem cell or plasma cell. Binding of an antagonist to CD19 may cause internalization of the CD19 antigen. The amino acid sequence of human CD19 is shown in The Leukocyte Antigen Facts Book, Barclay et al. supra, page 180, and also EMBL Genbank accession no. M28170 and Swissprot P11836.

As used herein, “B cell depletion” refers to a reduction in B cell levels in an animal or human after drug or antibody treatment, as compared to the level before treatment. B cell levels are measurable using well known assays such as by getting a complete blood count, by FACS analysis staining for known B cell markers, and by methods such as described in the Experimental Examples. B cell depletion can be partial or complete. In one embodiment, the depletion of CD20 expressing B cells is at least 25%. In a patient receiving a B cell depleting drug, B cells are generally depleted for the duration of time when the drug is circulating in the patient's body and the time for recovery of B cells.

Therefore, the term “amino acid” within the scope of the present invention is used in its broadest sense and is meant to include naturally occurring L alpha-amino acids or residues. The commonly used one and three letter abbreviations for naturally occurring amino acids are used herein (Lehninger, A. L., 1975, Biochemistry, 2d ed., pp. 71-92, Worth Publishers, New York). The correspondence between the standard single letter codes and the standard three letter codes is well known to the skilled artisan, and is reproduced here: A=Ala; C=Cys; D=Asp; E=Glu; F=Phe; G=Gly; H=His; I=lie; K=Lys; L=Leu; M=Met; N=Asn; P=Pro; Q=Gln; R=Arg; S=Ser; T=Thr; V=Val; W=Trp; Y=Tyr. The term includes D-amino acids as well as chemically modified amino acids such as amino acid analogs, naturally occurring amino acids that are not usually incorporated into proteins such as norleucine, and chemically synthesized compounds having properties known in the art to be characteristic of an amino acid. For example, analogs or mimetics of phenylalanine or proline, that allow the same conformational restriction of the peptide compounds as natural Phe or Pro, are included within the definition of amino acid. Such analogs and mimetics are referred to herein as “functional equivalents” of an amino acid. Other examples of amino acids are listed by Roberts and Vellaccio, 1983, In: The Peptides: Analysis, Synthesis, Biology, Gross and Meiehofer, eds., Vol. 5 p. 341, Academic Press, Inc., N.Y., which is incorporated herein by reference.

SABMs and TAs synthesized, for example, by standard solid phase synthesis techniques, are not limited to amino acids encoded by genes. Commonly encountered amino acids which are not encoded by the genetic code, include, for example, those described in International Publication No. WO 90/01940 such as, for example, 2-amino adipic acid (Aad) for Glu and Asp; 2-aminopimelic acid (Apm) for Glu and Asp; 2-aminobutyric (Abu) acid for Met, Leu, and other aliphatic amino acids; 2-aminoheptanoic acid (Ahe) for Met, Leu and other aliphatic amino acids; 2-aminoisobutyric acid (Aib) for Gly; cyclohexylalanine (Cha) for Val, and Leu and Ile; homoarginine (Har) for Arg and Lys; 2,3-diaminopropionic acid (Dpr) for Lys, Arg and His; N-ethylglycine (EtGly) for Gly, Pro, and Ala; N-ethylglycine (EtGly) for Gly, Pro, and Ala; N-ethylasparigine (EtAsn) for Asn, and Gln; Hydroxyllysine (Hyl) for Lys; allohydroxyllysine (AHyl) for Lys; 3-(and 4)-hydoxyproline (3Hyp, 4Hyp) for Pro, Ser, and Thr; allo-isoleucine (Alle) for Ile, Leu, and Val; ρ-amidinophenylalanine for Ala; N-methylglycine (MeGly, sarcosine) for Gly, Pro, and Ala; N-methylisoleucine (MeIle) for Ile; Norvaline (Nva) for Met and other aliphatic amino acids; Norleucine (Nle) for Met and other aliphatic amino acids; Ornithine (Orn) for Lys, Arg and His; Citrulline (Cit) and methionine sulfoxide (MSO) for Thr, Asn and Gln; N-methylphenylalanine (MePhe), trimethylphenylalanine, halo (F, Cl, Br, and I) phenylalanine, trifluorylphenylalanine, for Phe.

SABMs and TAs within the context of the present invention may be “engineered”, i.e., can be non-native or non-naturally occurring TAs. By “non-native” or “non-naturally occurring” is meant that the amino acid sequence of the particular SABM is not found in nature. That is to say, amino acid sequences of non-native or non-naturally occurring TAs or SABMs need not correspond to an amino acid sequence of a naturally occurring protein or polypeptide. TAs or SABMs of this variety may be produced or selected using a variety of techniques, including those well known to the skilled artisan. For example, constrained or unconstrained peptide libraries may be randomly generated and displayed on phage utilizing art standard techniques, for example, Lowman et al., 1998, Biochemistry 37:8870-8878.

SABMs and TAs and cytotoxic agents, when used within the context of the present invention, can be “conjugated” to eachother. The term “conjugated” is used in its broadest sense to encompass all methods of covalent attachment or joining that are known in the art. For example, in a typical embodiment, the SABM is a protein and the TA is an amino acid extension C- or N-terminus to the SABM. In addition, a short amino acid linker sequence may lie between the protein therapeutic and the SABM. In this scenario, the SABM, optional linker and TA will be encoded by a nucleic acid comprising a sequence encoding SABM operably linked (in the sense that the DNA sequences are contiguous and in reading frame) to an optional linker sequence encoding a short polypeptide as described below, and a sequence encoding the TA. In this typical scenario, the SABM is considered to be “conjugated” to the TA optionally via a linker sequence. In a related embodiment, the SABM amino acid sequence may interrupt or replace a section of the TA amino acid sequence, provided, of course, that the insertion of the SABM amino acid sequence does not interfere with the function of the protein therapeutic. In a further typical embodiment, the SABM will be linked, e.g., by chemical conjugation to the TA or other therapeutic optionally via a linker sequence. Typically, according to this embodiment, the SABM will be linked to the TA via a side chain of an amino acid somewhere in the middle of the TA that doesn't interfere with TA's ability to recognize the target activity. Here again, the SABM is considered to be “conjugated” to the TA.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

“Functional fragments”, of the antibodies of the invention comprise a portion of an intact antibody, generally including the antigen binding or variable region of the intact antibody or the Fc region of an antibody which retains FcR binding capability. Examples of antibody fragments include linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, each being directed against one or two antigenic site(s), typically one site. Furthermore, in contrast to conventional (polyclonal) antibody preparations which are derived from animals against an antigen so that several different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Methods of making chimeric antibodies are known in the art.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED® antibody wherein the antigen-binding region of the antibody is derived from an antibody produced by, e.g., immunizing macaque monkeys with the antigen of interest. Methods of making humanized antibodies are known in the art.

Human antibodies can also be produced using various techniques known in the art, including phage-display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985); Boerner et al., J. Immunol., 147(1):86-95 (1991).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD or IgM.

A “fusion protein” and a “fusion polypeptide” refer to a polypeptide having at least two portions covalently linked together, where each of the portions is a polypeptide having a different property. The property may be a biological property, such as activity in vitro or in vivo. The property may also be a simple chemical or physical property, such as binding to a target molecule, catalysis of a reaction, etc. The portions may be linked directly by a single peptide bond or through a peptide linker containing one or more amino acid residues. Generally, the portions and the linker will be in reading frame with each other.

An “isolate” polypeptide or antibody is one which has been identified and separated and/or recovered from a component of its natural environment. Contaminant components of its natural environment are materials which would interfere with diagnostic or therapeutic uses for the polypeptide or antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Humanized anti-ErbB2 (HER2) antibodies include huMAb4D5-1, huMAb4D5-2, huMAb4D5-3, huMAb4D5-4, huMAb4D5-5, huMAb4D5-6, huMAb4D5-7 and huMAb4D5-8 (HERCEPTIN7) as described in Table 3 of U.S. Pat. No. 5,821,337 expressly incorporated herein by reference; humanized 520C9 (WO93/21319) and humanized 2C4 antibodies as described in copending application Ser. No. 09/811115, and antibodies comprising the variable regions of anti-HER2 variants disclosed in WO 03/087131 and U.S. Patent Publication No. 2003/0228663, incorporated herein by reference. Throughout the disclosure, the terms “huMAb4D5-8” and “hu4D5-8” are used interchangeably.

The term “cytotoxic agent” as used herein refers to a substance that inhibits or prevents the function of cells and/or causes destruction of cells. The cytotoxic agent should be capable of being internalized and/or capable of inhibiting cell growth from outside the cell without necessarily binding to the cell surface. According to one preferred embodiment, the agent is a small molecule. According to another embodiment, the active portion of the cytotoxic agent is 1100 KD or less. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², B²¹³, P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin, or other intercalating agents, enzymes and fragments thereof such as nucleolytic enzymes, antibiotics, and toxins such as small molecule toxins or enzymatically active toxins of bacterial, fungal, plant or animal origin, (e.g., MMAE) including fragments and/or variants thereof, and the various antitumor or anticancer agents or grow inhibitory agents disclosed below. Other cytotoxic agents are described below. According to one preferred embodiment, the cytotoxic agent is not a radioisotope.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti- adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2- ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANETM Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluorometlhylomithine (DMFO); retinoids such as retinoic acid; capecitabine; oxaliplatin, including the oxaliplatin treatment regimen (FOLFOX); inhibitors of PKC-alpha, Raf, H-Ras, and EGFR (e.g., erlotinib (Tarceva™)) and pharmaceutically acceptable salts, acids or derivatives of any of the above.

A “growth inhibitory agent” when used herein refers to a compound or composition which inhibits growth of a cell in vitro and/or in vivo. Thus, the growth inhibitory agent may be one that significantly reduces the percentage of cells in S phase. Examples of growth inhibitory agents include agents that block cell cycle progression (at a place other than S phase), such as agents that induce GI arrest and M-phase arrest. Classical M-phase blockers include the vincas (vincristine and vinblastine), TAXOL® paclitaxel, and topo II inhibitors such as doxorubicin, epirubicin, daunorubicin, etoposide, and bleomycin. Those agents that arrest GI also spill over into S-phase arrest, for example, DNA alkylating agents such as tanoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antieioplastic drugs” by Murakaini et al. (W B Saunders: Philadelphia, 1995), especially p. 13.

Examples of “growth inhibitory” agents include an epidermal growth factor receptor (EGFR) antagonist (e.g., a tyrosine kinase inhibitor), HER1/EGFR inhibitor (e.g., erlotinib (Tarceva™), platelet derived growth factor inhibitors (e.g., Gleevec™ (Imatinib Mesylate)), a COX-2 inhibitor (e.g., celecoxib), and other bioactive and organic chemical agents, etc.

The term “therapeutically effective amount” refers to an amount of a conjugate molecule effective to “alleviate” or “treat” a disease or disorder in a subject. To the extent the conjugate molecule may prevent growth and/or kill existing cancer cells, it may be cytostatic and/or cytotoxic.

“Treatment” refers to amelioration or alleviation of a disease or disorder. Those in need of treatment include those already with the disorder as well as those in which the disorder is to be prevented. A subject is successfully “treated” for a cancer or an autoimmune disease if, after receiving a therapeutic amount of a conjugate according to the methods of the present invention, the subject shows observable and/or measurable reduction in or absence of one or more signs and symptoms of the particular disease. For example, for cancer, reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the specific cancer; reduced morbidity and mortality, and improvement in quality of life issues. Reduction of the signs or symptoms of a disease may also be felt by the patient. Treatment can achieve a complete response, defined as disappearance of all signs of cancer, or a partial response, wherein the size of the tumor is decreased, preferably by more than 50 percent, more preferably by 75%. A patient is also considered treated if the patient experiences stable disease. In a preferred embodiment, the cancer patients are still progression-free in the cancer after one year, preferably after 15 months. These parameters for assessing successful treatment and improvement in the disease are readily measurable by routine procedures familiar to a physician of appropriate skill in the art. In a preferred embodiment, the subject shows improvement from the illness while experiencing less side effects than a subject who may be treated received with the same conjugate molecule lacking the SABM.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain, as well as head and neck cancer, and associated metastases.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues.

B-cell regulated autoimmune diseases include arthritis (rheumatoid arthritis, juvenile rheumatoid arthritis, osteoarthritis, psoriatic arthritis), psoriasis, dermatitis including atopic dermatitis; chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), respiratory distress syndrome, adult respiratory distress syndrome (ARDS), meningitis, allergic rhinitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), lupus (including nephritis, non-renal, discoid, alopecia), juvenile onset diabetes, multiple sclerosis, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including ANCA), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, etc), graft versus host disease (GVHD), pemphigoid bullous, pemphigus (all including vulgaris, foliaceus), autoimmune polyendocrinopathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), autoimmune thrombocytopenia, autoimmune disease of the testis and ovary including autoimune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, Lymphoid interstitial pneumonitis (HIV), bronchiolitis obliterans (non-transplant) vs NSIP, Guillain-Barre' Syndrome, Large Vessel Vasculitis (including Polymyalgia Rheumatica and Giant Cell (Takayasu's) Arteritis), Medium Vessel Vasculitis (including Kawasaki's Disease and Polyarteritis Nodosa), ankylosing spondylitis, Berger's Disease (IgA nephropathy), Rapidly Progressive Glomerulonephritis, Primary biliary cirrhosis, Celiac sprue (gluten enteropathy), Cryoglobulinemia, ALS, coronary artery disease.

B cell neoplasms include CD20-positive Hodgkin's disease including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); Hairy cell leukemia. The non-Hodgkins lymphoma include low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS- related lymphoma and Waldenstrom's macroglobulinemia. Treatment of relapses of these cancers are also contemplated. LPHD is a type of Hodgkin's disease that tends to relapse frequently despite radiation or chemotherapy treatment and is characterized by CD20-positive malignant cells. CLL is one of four major types of leukemia. A cancer of mature B-cells called lymphocytes, CLL is manifested by progressive accumulation of cells in blood, bone marrow and lymphatic tissues. Indolent lymphoma is a slow-growing, incurable disease in which the average patient survives between six and 10 years following numerous periods of remission and relapse.

“Mammal” for purposes of treatment refers to any animal classified as a mammal, including humans, domestic and farm animals, and zoo, sports, or pet animals, such as dogs, horses, cats, cows, etc. Preferably, the mammal is human. The subject to be treated according to this invention is a mammal.

A “disorder” is any condition that would benefit from treatment with the compositions comprising the conjugate molecules of the invention. This includes chronic and acute disorders or diseases including those pathological conditions which predispose the mammal to the disorder in question.

“Elimination half-time” is used in its ordinary sense, as is described in Goodman and Gillman's The Pharmaceutical Basis of Therapeutics, pp. 21-25 Alfred Goodman Gilman, Louis S. Goodman, and Alfred Gilman, eds., 6th ed. 1980. Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2}, the time required for 50% completion of the process. The units of these two constants are time⁻¹ and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e. after drug absorption and distribution are complete). The half-time for drug elimination can be accurately determined from such a graph. According to one preferred embodiment of this invention, the conjugate molecules of this invention have a longer half-life and lower toxicity than conjugate molecules that lack the SABM.

“Transfection” refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell.

“Transformation” means introducing DNA into an organism so that the DNA is replicable, either as an extrachromosomal element or by chromosornal integrant. Depending on the host cell used, transformation is done using standard techniques appropriate to such cells. The calcium treatment employing calcium chloride, as described in section 1.82 of Sambrook et al., 1989, Molecular Cloning (2nd ed.), Cold Spring Harbor Laboratory, NY, is generally used for prokaryotes or other cells that contain substantial cell-wall barriers. Infection with Agrobacterium tumefaciens is used for transformation of certain plant cells, as described by Shaw et al., 1983 Gene, 23:315 and WO 89/05859, published 29 Jun. 1989. For mammalian cells without such cell walls, the calcium phosphate precipitation method described in sections 16.30-16.37 of Sambrook et al., supra, is preferred. General aspects of mammalian cell host system transformations have been described by Axel in U.S. Pat. No. 4,399,216, issued 16 Aug. 1983. Transformations into yeast are typically carried out according to the method of Van Solingen et al., 1977, J. Bact., 130:946 and Hsiao et al., 1979, Proc. Natl. Acad. Sci. (USA), 76:3829. However, other methods for introducing DNA into cells such as by nuclear injection, electroporation, or by protoplast fusion may also be used.

As used herein, the term “pulmonary administration” refers to administration of a formulation of the invention through the lungs by inhalation. As used herein, the term “inhalation” refers to intake of air to the alveoli. In specific examples, intake can occur by self-administration of a formulation of the invention while inhaling, or by administration via a respirator, e.g., to an patient on a respirator. The term “inhalation” used with respect to a formulation of the invention is synonymous with “pulmonary administration.”

As used herein, the term “parenteral” refers to introduction of a compound of the invention into the body by other than the intestines, and in particular, intravenous (i.v.), intraarterial (i.a.), intraperitoneal (i.p.), intrarnuscular (i.m.), intraventricular, and subcutaneous (s.c.) routes.

As used herein, the term “aerosol” refers to suspension in the air. In particular, aerosol refers to the particlization of a formulation of the invention and its suspension in the air. According to the present invention, an aerosol formulation is a formulation comprising a compound of the present invention that is suitable for aerosolization, i.e., particlization and suspension in the air, for inhalation or pulmonary administration.

II. MODES FOR CARRYING OUT THE INVENTION

A. SABM

SABMs within the context of the present invention bind albumin. Preferred SABMs that bind serum albumin include linear and cyclic peptides, preferably cyclic peptide compounds comprising the following formulae or are peptides that compete for binding serum albumin of a particular mammalian species with peptides of the following formulae:

| | |
|---|---|
| Xaa-Xaa-Cys-Xaa-Xaa-Xaa-Xaa-Xaa-Cys- | [SEQ ID NO: 1] |
| Xaa-Xaa-Phe-Cys-Xaa-Asp-Trp- | |
| Pro-Xaa-Xaa-Xaa-Ser-Cys | |
| Val-Cys-Tyr-Xaa-Xaa-Xaa-IIe-Cys-Phe | [SEQ ID NO: 2] |
| Cys-Tyr-Xaa1-Pro-Gly-Xaa-Cys | [SEQ ID NO: 3] |
| and | |
| Asp-Xaa-Cys-Leu-Pro-Xaa-Trp-Gly- | [SEQ ID NO: 4] |
| Cys-Leu-Trp | |

Preferred are peptide compounds of the foregoing general formulae comprising additional amino acids at the N-terminus (Xaa)ₓ and additional amino acids at the C-terminus (Xaa)_{z}, wherein Xaa is an amino acid and x and z are a whole number greater or equal to 0 (zero), generally less than 100, preferably less than 10 and more preferably 0, 1, 2, 3, 4 or 5 and more preferably 4 or 5 and wherein Xaa₁ is selected from the group consisting of Ile, Phe, Tyr and Val.

Further preferred SABMs that bind a serum albumin are identified as described herein in the context of the following general formulae:
Trp-Cys-Asp-Xaa-Xaa-Leu-Xaa-Ala-Xaa-Asp-Leu-Cys (SEQ ID NO: 5) and Asp-Leu-Val-Xaa-Leu-Gly-Leu-Glu-Cys-Trp [SEQ ID NO: 6]
where additional amino acids may be present at the N-terminal end (Xaa)ₓ and additional amino acids may be present at the C-terminal end (Xaa)_{z}, and where Xaa is an amino acid and x and z are a whole number greater or equal to zero, generally less than 100, preferably less than 10 and more preferably 0, 1, 2, 3, 4 or 5 and more preferably 4 or 5.

According to this aspect of the invention reference is made to the Examples below and particularly the Tables contained therein showing especially exemplary peptides and appropriate amino acids for selecting peptides ligands that bind a mammalian serum albumin. In a preferred aspect, reference is made to Table 7 for selecting SABMs that bind across several species of serum albumin.

Preferred compounds according to this aspect of the invention include:

| | |
|---|---|
| DLCLRDWGCLW | (SEQ ID NO:7) |
| DICLPRWGCLW | (SEQ ID NO:8) |
| MEDICLPRWGCLWGD | (SEQ ID NO:9) |
| QRLMEDICLPRWGCLWEDDE | (SEQ ID NO:10) |
| QGLIGDICLPRWGCLWGRSV | (SEQ ID NO:11) |
| QGLIGDICLPRWGCLWGRSVK | (SEQ ID NO:12) |
| EDICLPRWGCLWEDD | (SEQ ID NO:13) |
| RLMEDICLPRWGCLWEDD | (SEQ ID NO:14) |
| MEDICLPRWGCLWEDD | (SEQ ID NO:15) |
| MEDICLPRWGCLWED | (SEQ ID NO:16) |
| RLMEDICLARWGCLWEDD | (SEQ ID NO:17) |
| EVRSFCTRWPAEKSCKPLRG | (SEQ ID NO:18) |
| RAPESFVCYWETICFERSEQ | (SEQ ID NO:19) |
| EMCYFPGICWM | (SEQ ID NO:20) |

In a preferred embodiment, SABMs of the present invention bind human serum albumin and can be identified by their ability to compete for binding of human serum albumin in an in vitro assay with SABMs having the general formulae shown below, where additional amino acids may be present at the N-terminal end (Xaa)ₓ and at the C-terminal end (Xaa)_{z}:

| | |
|---|---|
| D X C L P X W G C L W | (SEQ ID NO:4) |
| F C X D W P X X X S C | (SEQ ID NO:1) |
| V C Y X X X I C F | (SEQ ID NO:2) |
| C Y X₁ P G X C X | (SEQ ID NO:3) |

where Xaa is an amino acid, x and z are preferably 4 or 5, and Xaa₁ is selected from the group consisting of Ile, Phe, Tyr, and Val.

In particular embodiments, the SABMs of the present invention will compete with any of the SABMs represented in SEQ ID NO: 7-20 described herein above and preferably will compete with SEQ ID NO: 10 for binding human serum albumin.

As will be appreciated from the foregoing, the term “compete” and “ability to compete” are relative terms. Thus the terms, when used to describe the SABMs of the present invention, refer to SABMs that produce a 50% inhibition of binding of, for example the peptide represented by SEQ ID NO: 10, when present at 50 μM, preferably when present at 1 μM, more preferably 100 nM, and preferably when present at 1 nM or less in a standard competition assay as described herein. However, SABMs having an affinity for a serum albumin of less than about 1 nM and preferably between about 1 pM and 1 nM are equally likely to be SABMs within the context of the present invention.

For in vitro assay systems to determine whether a peptide or other compound has the “ability” to compete with a SABM for binding to serum albumin as noted herein, the skilled artisan can employ any of a number of standard competition assays. Competitive binding assays rely on the ability of a labeled standard to compete with the test sample analyte for binding with a limited amount of ligand. The amount of analyte in the test sample is inversely proportional to the amount of standard that becomes bound to the ligand.

Thus, the skilled artisan may determine whether a peptide or other compound has the ability to compete with a SABM for binding to albumin employing procedures that include, but are not limited to, competitive assay systems using techniques such as radioimmunoassays (RIA), enzyme immunoassays (EIA), preferably the enzyme linked immunosorbent assay (ELISA), “sandwich” immunoassays, immunoradiometric assays, fluorescent immunoassays, and immunoelectrophoresis assays, to name but a few.

For these purposes, the selected SABM will be labeled with a detectable moiety (the detectably labeled SABM hereafter called the “tracer”) and used in a competition assay with a candidate compound for binding albumin. Numerous detectable labels are available that can be preferably grouped into the following categories:

(a) Radioisotopes, such as ³⁵S, ¹⁴C, ¹²⁵I, ³H, and ¹³¹I. The SABM can be labeled with the radioisotope using techniques described in Coligen et al., 1991, eds., Current Protocols in Immunology, Volumes 1 and 2, Wiley-Interscience, New York, N.Y., for example. Radioactivity can be measured using scintillation counting.

(b) Fluorescent labels such as rare earth chelates (europium chelates) or fluorescein and its derivatives, rhodamine and its derivatives, dansyl, lissamine, phycoerythrin, and Texas Red are available. The fluorescent labels can be conjugated to the peptide compounds using the techniques disclosed in Current Protocols in Immunology, supra, for example. Fluorescence can be quantified using a fluorimeter.

(c) Various enzyme-substrate labels are available and U.S. Pat. No. 4,275,149 provides a review of some of these. The enzyme preferably catalyzes a chemical alteration of the chromogenic substrate that can be measured using various techniques. For example, the enzyme may catalyze a color change in a substrate, that can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example:

(i) Horseradish peroxidase (HRP) with hydrogen peroxidase as a substrate, where the hydrogen peroxidase oxidizes a dye precursor (e.g. ABTS, orthophenylene diamine (OPD) or 3,3′,5,5′-tetramethyl benzidine hydrochloride (TMB));

(ii) alkaline phosphatase (AP) with para-nitrophenyl phosphate as chromogenic substrate; and

(iii) β-D-galactosidase (β-D-Gal) with a chromogenic substrate (e.g. p-nitrophenyl-⊖-D-galactosidase) or fluorogenic substrate 4-methylumbelliferyl-β-D-galactosidase.

According to a particular assay, the tracer is incubated with immobilized target in the presence of varying concentrations of unlabeled candidate compound. Increasing concentrations of successful candidate compound effectively compete with binding of the tracer to immobilized target. The concentration of unlabeled candidate compound at which 50% of the maximally-bound tracer is displaced is referred to as the “IC₅₀” and reflects the IgG binding affinity of the candidate compound. Therefore a candidate compound with an IC₅₀ of 1 mM displays a substantially weaker interaction with the target than a candidate compound with an IC₅₀ of 1 iM.

In some phage display ELISA assays, binding affinity of a mutated (“mut”) sequence was directly compared of a control (“con”) peptide using methods described in Cunningham et al., 1994, EMBO J. 13:2508, and characterized by the parameter EC₅₀. Assays were performed under conditions where EC₅₀(con)/EC₅₀(mut) will approximate K_{d}(con)/K_{d}(mut).

Accordingly, the invention provides compounds “having the ability to compete” for albumin such as human serum albumin binding in an in vitro assay as described. Preferably the compound has an IC₅₀ for the target such as human serum albumin of less than 1 iM. Preferred among these compound are compounds having an IC₅₀ of less than about 100 nM, and preferably less than about 10 nM or less than about 1 nM. In further preferred embodiments according to this aspect of the invention the compounds display an IC₅₀ for the target molecule such as or human serum albumin of less than about 100 pM and more preferably less than about 10 pM.

A preferred in vitro assay for the determination of a candidate compound's ability to compete with a SABM described herein is as follows and is described more fully in the Examples. In preferred embodiments the candidate compound is a peptide. The ability of a candidate compound to compete with a labeled SABM tracer for binding to human serum albumin is monitored using an ELISA. Dilutions of a candidate compound in buffer are added to microtiter plates coated with human serum albumin (as described in the Example Sections) along with tracer for 1 hour. The microtiter plate is washed with wash buffer and the amount of tracer bound to human serum albumin measured.

B. SABM:TA:Cvtotoxic Agent Combinations

The SABM is linked to a TA:cytotoxic agent to form a conjugate molecule that comprises at least one of each component (i.e., at least three different components). Each component can be optionally joined to each other via a flexible linker domain.

Depending on the type of linkage and its method of production, the SABM domain may be joined via its N- or C-terminus to the N- or C-terminus of the TA. For example, when preparing the conjugate molecules of the present invention via recombinant techniques, nucleic acid encoding a SABM will be operably linked to nucleic acid encoding the TA sequence, optionally via a linker domain. Typically the construct encodes a fusion protein wherein the C-terminus of the SABM is joined to the N-terminus of the TA. However, especially when synthetic techniques are employed, fusions where, for example, the N-terminus of the SABM is joined to the N- or C-terminus of the TA also are possible.

In some instances, the SABM domain may be inserted within the TAs molecule rather than being joined to the TAs at its N-or C-terminus. This configuration may be used to practice the invention so long as the functions of the SABM domain and the TAs are preserved. For example, a SABM may be inserted into a non-binding light chain CDR of an immunoglobulin without interfering with the ability of the immunoglobulin to bind to its target. Regions of TAs molecules that can accommodate SABM domain insertions may be identified empirically (i.e., by selecting an insertion site, randomly, and assaying the resulting conjugate for the function of the TAs), or by sequence comparisons amongst a family of related TAs molecules (e.g., for TAs s that are proteins) to locate regions of low sequence homology. Low sequence homology regions are more likely to tolerate insertions of SABMs domains than are regions that are well-conserved. For TAs whose three-dimensional structures are known (e.g. from X-ray crystallographic or NMR studies), the three-dimensional structure may provide guidance as to SABM insertion sites. For example, loops or regions with high mobility (i.e., large temperature or “B” factors) are more likely to accommodate SABM domain insertions than are highly ordered regions of the structure, or regions involved in ligand binding or catalysis.

C. Linker Domains

The SABM domain is optionally linked to the TAs via a linker. The linker component of the conjugate molecule of the invention does not necessarily participate, but may contribute to the function of the conjugate molecule. Therefore, the linker domain is defined as any group of molecules that provides a spatial bridge between the TAs and the SABM domain.

The linker domain can be of variable length and makeup, however, it is the length of the linker domain and not its structure that is important for creating the spatial bridge. The linker domain preferably allows for the SABM of the conjugate molecule to bind, substantially free of steric and/or conformational restrictions to the target molecule. Therefore, the length of the linker domain is dependent upon the character of the two “functional” domains of the conjugate molecule, ie., the SABM and the TAs.

One skilled in the art will recognize that various combinations of atoms provide for variable length molecules based upon known distances between various bonds. See, for example, Morrison and Boyd, 1997, Organic Chemistry, 3rd Ed., Allyn and Bacon, Inc., Boston, Mass. The linker domain may be a polypeptide of variable length. The amino acid composition of the polypeptide determines the character and length of the linker. In a preferred embodiment, the linker molecule comprises a flexible, hydrophilic polypeptide chain. Exemplary linker domains comprise one or more Gly and/or Ser residues, such as those described in the Example sections below.

D. Recombinant Synthesis

The present invention encompasses a composition of matter comprising an isolated nucleic acid, preferably DNA, encoding a SABM or a conjugate molecule comprising a SABM and a polypeptide TAs as described herein. DNAs encoding the peptides of the invention can be prepared by a variety of methods known in the art. These methods include, but are not limited to, chemical synthesis by any of the methods described in Engels et al. 1989, Agnew. Chem. Int. Ed. Engl. 28:716-734 (the entire disclosure of which is incorporated herein by reference) such as the triester, phosphite, phosphoramidite, and H-phosphonate chemical synthesis methods. In one embodiment, codons preferred by the expression host cell are used in the design of the encoding DNA. Alternatively, DNA encoding the peptides can be altered to encode one or more variants by using recombinant DNA techniques, such as site specific mutagenesis (Kunkel et al., 1991, Methods Enzymol., 204:125-139; Carter et al. 1986, Nucl. Acids Res. 13:4331; Zoller et al. 1982, Nucl. Acids Res. 10:6487), cassette mutagenesis (Wells et al. 1985, Gene 34:315), restriction selection mutagenesis (Carter, 1991, In: Directed Mutagenesis: A Practical Approach, M. J. McPherson, ed., IRL Press, Oxford), and the like.

According to preferred aspects described above, the nucleic acid encodes a SABM capable of binding a target molecule. Target molecules include, for example, extracellular molecules such as various serum factors, including but not limited to, plasma proteins such as serum albumin, immunoglobulins, apolipoproteins or transferrin, or proteins found on the surface of erythrocytes or lymphocytes, provided, of course, that binding of the SABM to the cell surface protein does not substantially interfere with the normal function of the cell. Preferred for use in the present invention are SABMs that bind serum albumin with a desired affinity, for example, with high affinity, or with an affinity that facilitates useful tissue uptake and diffusion of a bioactive molecule that is fused to the SABM.

According to another preferred aspect of the invention, the nucleic acid encodes a conjugate molecule comprising a SABM sequence and an TAs. In this aspect of the invention, the TAs may comprise any polypeptide compound useful as a therapeutic or diagnostic agent, e.g., enzymes, hormones, cytokines, antibodies, or antibody fragments. The nucleic acid molecule according to this aspect of the present invention encodes a conjugate molecule and the nucleic acid encoding the SABM sequence is operably linked to (in the sense that the DNA sequences are contiguous and in reading frame) the nucleic acid encoding the biologically active agent. Optionally these DNA sequences may be linked through a nucleic acid sequence encoding a linker domain amino acid sequence.

According to this aspect, the invention further comprises an expression control sequence operably linked to the DNA molecule encoding a peptide of the invention, an expression vector, such as a plasmid, comprising the DNA molecule, where the control sequence is recognized by a host cell transformed with the vector, and a host cell transformed with the vector. In general, plasmid vectors contain replication and control sequences derived from species compatible with the host cell. The vector ordinarily carries a replication site, as well as sequences that encode proteins capable of providing phenotypic selection in transformed cells.

For expression in prokaryotic hosts, suitable vectors include pBR322 (ATCC No. 37,017), phGH107 (ATCC No. 40,011), pBO475, pS0132, pRIT5, any vector in the pRIT20 or pRIT30 series (Nilsson and Abrahmsen 1990, Meth. Enzymol. 185:144-161), pRIT2T, pKK233-2, pDR540, and pPL-lambda. Prokaryotic host cells containing the expression vectors of the present invention include E. coli K12 strain 294 (ATCC NO. 31,446), E. coli strain JM11 (Messing et al. 1981, Nucl. Acid Res. 9:309), E. coli strain B, E. coli Strain_{—}1776 (ATCC No. 31537), E. coli c600, E. coli W3110 (F-, gamma-, prototrophic, ATCC No. 27,325), E. coli strain 27C7 (W3110, tonA, phoA E15, (argF-lac)169, ptr3, degP41, ompT, kan^{r}) (U.S. Pat. No. 5,288,931, ATCC No. 55,244), Bacillus subtilis, Salmonella typhimurium, Serratia marcesans, and Pseudomonas species.

In addition to prokaryotes, eukaryotic organisms, such as yeasts, or cells derived from multicellular organisms can be used as host cells. For expression in yeast host cells, such as common baker's yeast or Saccharomyces cerevisiae, suitable vectors include episomally-replicating vectors based on the 2-micron plasmid, integration vectors, and yeast artificial chromosome (YAC) vectors. For expression in insect host cells, such as Sf9 cells, suitable vectors include baculoviral vectors. For expression in plant host cells, particularly dicotyledonous plant hosts, such as tobacco, suitable expression vectors include vectors derived from the Ti plasmid of Agrobacterium tumefaciens.

Examples of useful mammalian host cells include monkey kidney CV1 line transformed by SV40 (COS-7, ATCC.CRL 1651); human embryonic kidney line (293 or 293 cells subcloned for growth in suspension culture, Graham et al. 1977, J. Gen Virol. 36:59); baby hamster kidney cells (BHK, ATCC CCL 10); Chinese hamster ovary cells/-DHFR (CHO, Urlaub and Chasin 1980, Proc. Natl. Acad. Sci. USA, 77:4216); mouse sertoli cells (TM4, Mather 1980, Biol. Reprod. 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al 1982, Annals N.Y. Acad. Sci. 383:44-68); MRC 5 cells; FS4 cells; and a human hepatoma cell line (Hep G2). For expression in mammalian host cells, useful vectors include vectors derived from SV40, vectors derived from cytomegalovirus such as the pRK vectors, including pRK5 and pRK7 (Suva et al. 1987, Science 237:893-896; EP 307,247 (Mar. 15, 1989), EP 278,776 (Aug. 17, 1988)) vectors derived from vaccinia viruses or other pox viruses, and retroviral vectors such as vectors derived from Moloney's murine leukemia virus (MoMLV).

Optionally, DNA encoding the peptide of interest is operably linked to a secretory leader sequence resulting in secretion of the expression product by the host cell into the culture medium. Examples of secretory leader sequences include STII, ecotin, lamB, herpes GD, 1 pp, alkaline phosphatase, invertase, and alpha factor. Also suitable for use herein is the 36 amino acid leader sequence of protein A (Abrahmsen et al. 1985, EMBO J. 4:3901).

Host cells are transfected and preferably transformed with the above-described expression or cloning vectors of this invention and cultured in conventional nutrient media modified as appropriate for inducing promoters, selecting transformants, or amplifying the genes encoding the desired sequences.

Prokaryotic host cells used to produce the present peptides can be cultured as described generally in Sambrook et al., supra.

The mammalian host cells used to produce peptides of the invention can be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in the art (for example, Ham and Wallace, 1979, Meth. Enz. 58:44; Barnes and Sato 1980, Anal. Biochem. 102:255, U.S. Pat. Nos. 4,767,704; 4,657,866; 4,927,762; or 4,560,655; WO 90/03430; WO 87/00195; U.S. Pat. Re. 30,985; or U.S. Pat. No. 5,122,469, the disclosure of each is incorporated herein by reference) may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The host cells referred to in this disclosure encompass cells in in vitro culture as well as cells that are within a host animal.

E. Chemical Synthesis Another method of producing the SABMs of the invention involves chemical synthesis. This can be accomplished by using methodologies well known in the art (see Kelley and Winkler, 1990, In: Genetic Engineering Principles and Methods, Setlow, J. K, ed., Plenum Press, N.Y., Vol. 12, pp 1-19; Stewart, et al., 1984, J. M. Young, J. D., Solid Phase Peptide Synthesis, Pierce Chemical Co., Rockford, Ill. See also U.S. Pat. Nos. 4,105,603; 3,972,859; 3,842,067; and 3,862,925).

SABMs of the invention can be prepared conveniently using solid-phase peptide synthesis. Merrifield, 1964, J. Am. Chem. Soc. 85:2149; Houghten, 1985, Proc. Natl. Acad. Sci. USA 82:5132. Solid-phase peptide synthesis also can be used to prepare the conjugate molecule compositions of the invention if the TAs is or comprises a polypeptide.

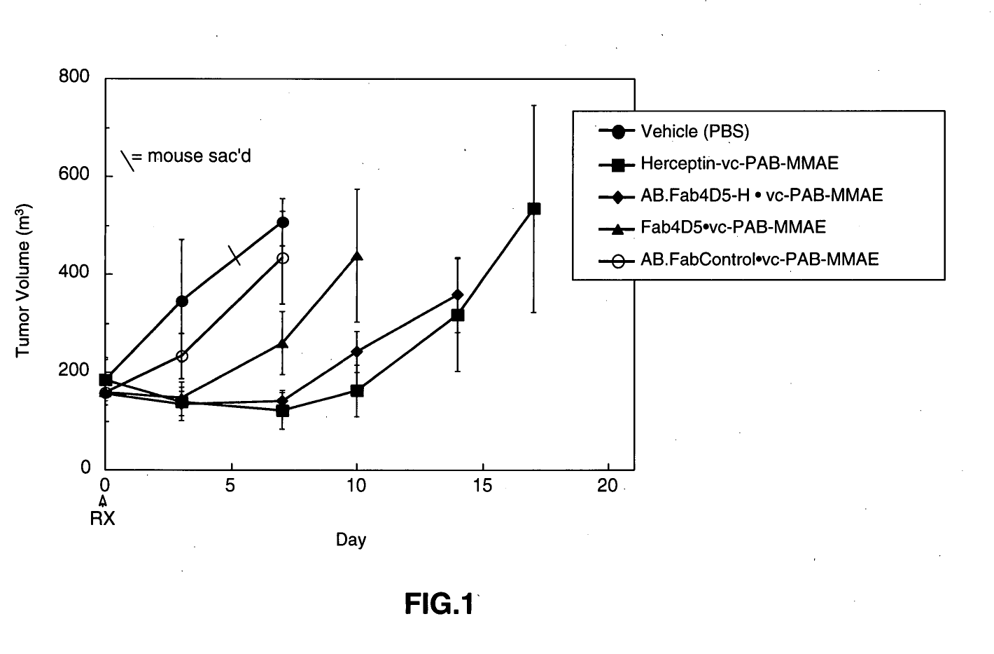

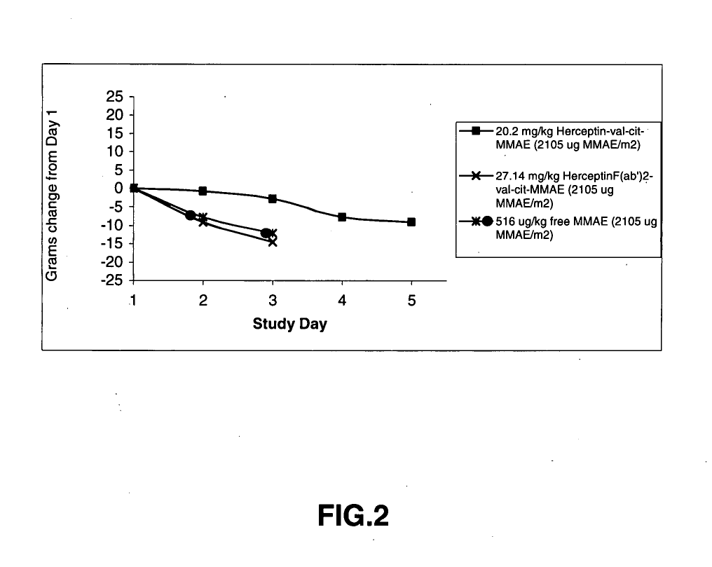

Solid-phase synthesis begins at the carboxy terminus of the nascent peptide by coupling a protected amino acid to an inert solid support. The inert solid support can be any macromolecule capable of serving as an anchor for the C-terminus of the initial amino acid. Typically, the macromolecular support is a cross-linked polymeric resin (e.g., a polyamide or polystyrene resin) as shown in FIGS. 1-1 and 1-2, on pages 2 and 4 of Stewart and Young, supra. In one embodiment, the C-terminal amino acid is coupled to a polystyrene resin to form a benzyl ester. A macromolecular support is selected such that the peptide anchor link is stable under the conditions used to deprotect the alpha-amino group of the blocked amino acids in peptide synthesis. If a base-labile alpha-protecting group is used, then it is desirable to use an acid-labile link between the peptide and the solid support. For example, an acid-labile ether resin is effective for base-labile Fmoc-amino acid peptide synthesis as described on page 16 of Stewart and Young, supra. Alternatively, a peptide anchor link and α-protecting group that are differentially labile to acidolysis can be used. For example, an aminomethyl resin such as the phenylacetamidomethyl (Pam) resin works well in conjunction with Boc-amino acid peptide synthesis as described on pages 11-12 of Stewart and Young, supra.

After the initial amino acid is coupled to an inert solid support, the alpha-amino protecting group of the initial amino acid is removed with, for example, trifluoroacetic acid (TFA) in methylene chloride and neutralized in, for example, triethylamine (TEA). Following deprotection of the initial amino acid's alpha-amino group, the next alpha-amino and side chain protected amino acid in the synthesis is added. The remaining alpha-amino and, if necessary, side chain protected amino acids are then coupled sequentially in the desired order by condensation to obtain an intermediate compound connected to the solid support. Alternatively, some amino acids may be coupled to one another to form a fragment of the desired peptide followed by addition of the peptide fragment to the growing solid phase peptide chain.

The condensation reaction between two amino acids, or an amino acid and a peptide, or a peptide and a peptide can be carried out according to the usual condensation methods such as the axide method, mixed acid anhydride method, DCC (N,N′-dicyclohexylcarbodiimide) or DIC (N,N′-diisopropylcarbodiimide) methods, active ester method, p-nitrophenyl ester method, BOP (benzotriazole-1-yl-oxy-tris [dimethylamino] phosphonium hexafluorophosphate) method, N-hydroxysuccinic acid imido ester method, etc., and Woodward reagent K method.

It is common in the chemical synthesis of peptides to protect any reactive side chain groups of the amino acids with suitable protecting groups. Ultimately, these protecting groups are removed after the desired polypeptide chain has been sequentially assembled. Also common is the protection of the alpha-amino group on an amino acid or peptide fragment while the C-terminal carboxy group of the amino acid or peptide fragment reacts with the free N-terminal amino group of the growing solid phase polypeptide chain, followed by the selective removal of the alpha-amino group to permit the addition of the next amino acid or peptide fragment to the solid phase polypeptide chain. Accordingly, it is common in polypeptide synthesis that an intermediate compound is produced that contains each of the amino acid residues located in the desired sequence in the peptide chain wherein individual residues still carry side-chain protecting groups. These protecting groups can be removed substantially at the same time to produce the desired polypeptide product following removal from the solid phase.

Alpha- and epsilon-amino side chains can be protected with benzyloxycarbonyl (abbreviated Z), isonicotinyloxycarbonyl (iNOC), o-chlorobenzyloxycarbonyl [Z(2Cl)], p-nitrobenzyloxycarbonyl [Z(NO₂)], p-methoxybenzyloxycarbonyl [Z(OMe)], t-butoxycarbonyl (Boc), t-amyloxycarbonyl (Aoc), isobornyloxycarbonyl, adamantyloxycarbonyl, 2-(4-biphenyl)-2-propyloxycarbonyl (Bpoc), 9-fluorenylmethoxycarbonyl (Fmoc), methylsulfonyethoxycarbonyl (Msc), trifluoroacetyl, phthalyl, formyl, 2-nitrophenylsulphenyl (NPS), diphenylphosphinothioyl (Ppt), and dimethylphosphinothioyl (Mpt) groups, and the like.

Protective groups for the carboxy functional group are exemplified by benzyl ester (OBzl), cyclohexyl ester (Chx), 4-nitrobenzyl ester (ONb), t-butyl ester (Obut), 4-pyridylmethyl ester (OPic), and the like. It is often desirable that specific amino acids such as arginine, cysteine, and serine possessing a functional group other than amino and carboxyl groups are protected by a suitable protective group. For example, the guanidino group of arginine may be protected with nitro, p-toluenesulfonyl, benzyloxycarbonyl, adamantyloxycarbonyl, p-methoxybenzesulfonyl, 4-methoxy-2,6-dimethylbenzenesulfonyl (Nds), 1,3,5-trimethylphenysulfonyl (Mts), and the like. The thiol group of cysteine can be protected with p-methoxybenzyl, trityl, and the like.

Many of the blocked amino acids described above can be obtained from commercial sources such as Novabiochem (San Diego, Calif.), Bachem C A (Torrence, Calif.) or Peninsula Labs (Belmont, Calif.).

Stewart and Young, supra, provides detailed information regarding procedures for preparing peptides. Protection of alpha-amino groups is described on pages 14-18, and side chain blockage is described on pages 18-28. A table of protecting groups for amine, hydroxyl, and sulfhydryl functions is provided on pages 149-151.

After the desired amino acid sequence has been completed, the peptide can be cleaved away from the solid support, recovered, and purified. The peptide is removed from the solid support by a reagent capable of disrupting the peptide-solid phase link, and optionally deprotects blocked side chain functional groups on the peptide. In one embodiment, the peptide is cleaved away from the solid phase by acidolysis with liquid hydrofluoric acid (HF), which also removes any remaining side chain protective groups. Preferably, in order to avoid alkylation of residues in the peptide (for example, alkylation of methionine, cysteine, and tyrosine residues), the acidolysis reaction mixture contains thio-cresol and cresol scavengers. Following HF cleavage, the resin is washed with ether, and the free peptide is extracted from the solid phase with sequential washes of acetic acid solutions. The combined washes are lyophilized, and the peptide is purified.

F. Chemical Conjugation of Conjugate Molecules

In certain embodiments, the conjugate molecules may comprise TAs that are organic compounds having diagnostic or therapeutic utility, or alternatively, fusions between a SABM and a polypeptide TAs in configurations that cannot be encoded in a single nucleic acid. Examples of the latter embodiment include fusions between the amino terminus of a SABM and the amino terminus of the TAs, or fusions between the carboxy-terminus of a SABM and the carboxy-terminus of the TAs.

Chemical conjugation may be employed to prepare these embodiments of the conjugate molecule, using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene, 2,6-diisocyanate), and bis-active fluorine compounds (such as 1,5-difluoro-2,4-dinitrobenzene). Methods useful for conjugating cytotoxic agentss to polypeptides such as antibodies are known.

G. Disulfide-Linked Peptides

As described above, some embodiments of the invention include cyclized SABMs. SABMs may be cyclized by formation of a disulfide bond between cysteine residues. Such peptides can be made by chemical synthesis as described above and then cyclized by any convenient method used in the formation of disulfide linkages. For example, peptides can be recovered from solid phase synthesis with sulfhydryls in reduced form, dissolved in a dilute solution wherein the intramolecular cysteine concentration exceeds the intermolecular cysteine concentration in order to optimize intramolecular disulfide bond formation, such as a peptide concentration of 25 mM to 1 uM, and preferably 500 uM to 1 uM, and more preferably 25 uM to 1 uM, and then oxidized by exposing the free sulfhydryl groups to a mild oxidizing agent that is sufficient to generate intramolecular disulfide bonds, e.g., molecular oxygen with or without catalysts such as metal cations, potassium ferricyanide, sodium tetrathionate, and the like. Alternatively, the peptides can be cyclized as described in Pelton et al., 1986, J. Med. Chem. 29:2370-2375.

Cyclization can be achieved by the formation, for example, of a disulfide bond or a lactam bond between a first and a second residue capable of forming a disulfide bond, for example, Cys, Pen, Mpr, and Mpp and its 2-amino group-containing equivalents. Residues capable of forming a lactam bridge include, for example, Asp, Glu, Lys, Orn, αβ-diaminobutyric acid, diaminoacetic acid, aminobenzoic acid, and mercaptobenzoic acid. The compounds herein can be cyclized for example via a lactam bond that can utilize the side chain group of a non-adjacent residue to form a covalent attachment to the N-terminus amino group of Cys or other amino acid. Alternative bridge structures also can be used to cyclize the compounds of the invention, including for example, peptides and peptidomimetics, that can cyclize via S—S, CH₂—S, CH₂—O—CH₂, lactam ester or other linkages.

H. Pharmaceutical Compositions

Pharmaceutical compositions which comprising the conjugate molecules of the invention may be administered in any suitable manner, including parental, topical, oral, or local (such as aerosol or transdermal), or any combination thereof.

Other suitable compositions of the present invention comprise any of the conjugate molecules noted above with a pharmaceutically acceptable carrier. The nature of the carrier differs with the mode of administration. For example, for oral administration, a solid carrier is preferred; for i.v. administration, a liquid salt solution carrier is generally used.

The compositions of the present invention include pharmaceutically acceptable components that are compatible with the subject and the protein of the invention. These generally include suspensions, solutions, and elixirs, and most especially biological buffers, such as phosphate buffered saline, saline, Dulbecco's Media, and the like. Aerosols may also be used, or carriers such as starches, sugars, microcrystalline cellulose, diluents, granulating agents, lubricants, binders, disintegrating agents, and the like (in the case of oral solid preparations, such as powders, capsules, and tablets).

As used herein, the term “pharmaceutically acceptable” generally means approved by a regulatory agency of the Federal or a state government or listed in the U.S. Pharmacopeia or other generally recognized pharmacopeia for use in animals, and more particularly in humans.

The formulation of choice can be accomplished using a variety of the aforementioned buffers, or even excipients including, for example, pharmaceutical grades of mannitol, lactose, starch, magnesium stearate, sodium saccharin cellulose, magnesium carbonate, and the like. “PEGylation” of the compositions may be achieved using techniques known to the art (see for example International Patent Publication No. WO92/16555, U.S. Pat. No. 5,122,614 to Enzon, and International Patent Publication No. WO92/00748).

A preferred route of administration of the present invention is in the aerosol or inhaled form. The compounds of the present invention, combined with a dispersing agent or dispersant, can be administered in an aerosol formulation as a dry powder or in a solution or suspension with a diluent.

As used herein, the term “dispersant” refers to an agent that assists aerosolization of the compound or absorption of the protein in lung tissue, or both. Preferably the dispersant is pharmaceutically acceptable. Suitable dispersing agents are well known in the art, and include but are not limited to surfactants and the like. For example, surfactants that are generally used in the art to reduce surface induced aggregation of a compound, especially a peptide compound, caused by atomization of the solution forming the liquid aerosol, may be used. Nonlimiting examples of such surfactants are surfactants such as polyoxyethylene fatty acid esters and alcohols, and polyoxyethylene sorbitan fatty acid esters. Amounts of surfactants used will vary, being generally within the range of from about 0.001% to about 4% by weight of the formulation. In a specific aspect, the surfactant is polyoxyethylene sorbitan monooleate or sorbitan trioleate. Suitable stirfactants are well known in the art, and can be selected on the basis of desired properties, depending on the specific formulation, concentration of the compound, diluent (in a liquid formulation) or form of powder (in a dry powder formulation), and the like.

Moreover, depending on the choice of the conjugate molecule, the desired therapeutic effect, the quality of the lung tissue (e.g., diseased or healthy lungs), and numerous other factors, the liquid or dry formulations can comprise additional components, as discussed further below.

The liquid aerosol formulations generally contain the conjugate molecules and a dispersing agent in a physiologically acceptable diluent. The dry powder aerosol formulations of the present invention consist of a finely divided solid form of the conjugate molecule and a dispersing agent. With either the liquid or dry powder aerosol formulation, the formulation must be aerosolized. That is, it must be broken down into liquid or solid particles in order to ensure that the aerosolized dose actually reaches the alveoli. In general the mass median dynamic diameter will be 5 micrometers or less in order to ensure that the drug particles reach the lung alveoli (Wearley, 1991, Crit. Rev. in Ther. Drug Carrier Systems 8:333). The term “aerosol particle” is used herein to describe the liquid or solid particle suitable for pulmonary administration, i.e., that will reach the alveoli. Other considerations such as construction of the delivery device, additional components in the formulation and particle characteristics are important. These aspects of pulmonary administration of a drug are well known in the art, and manipulation of formulations, aerosolization means and construction of a delivery device require at most routine experimentation by one of ordinary skill in the art.

With regard to construction of the delivery device, any form of aerosolization known in the art, including but not limited to nebulization, atomization or pump aerosolization of a liquid formulation, and aerosolization of a dry powder formulation, can be used in the practice of the invention. A delivery device that is uniquely designed for administration of solid formulations is envisioned. Often, the aerosolization of a liquid or a dry powder formulation will require a propellant. The propellant may be any propellant generally used in the art. Specific nonlimiting examples of such useful propellants are a chloroflourocarbon, a hydrofluorocarbon, a hydochlorofluorocarbon, or a hydrocarbon, including triflouromethane, dichlorodiflouromethane, dichlorotetrafuoroethanol, and 1,1,1,2-tetraflouroethane, or combinations thereof.

In a preferred aspect of the invention, the device for aerosolization is a metered dose inhaler. A metered dose inhaler provides a specific dosage when administered, rather than a variable dose depending on administration. Such a metered dose inhaler can be used with either a liquid or a dry powder aerosol formulation. Metered dose inhalers are well known in the art.

Once the conjugate molecule reaches the lung, a number of formulation-dependent factors affect the drug absorption. It will be appreciated that in treating a disease or disorder that requires circulatory levels of the compound, such factors as aerosol particle size, aerosol particle shape, the presence or absence of infection, lung disease or emboli may affect the absorption of the compounds. For each of the formulations described herein, certain lubricators, absorption enhancers, protein stabilizers or suspending agents may be appropriate. The choice of these additional agents will vary depending on the goal. It will be appreciated that in instances where local delivery of the compounds is desired or sought, such variables as absorption enhancement will be less critical.

I. Liquid Aerosol Formulations

The liquid aerosol formulations of the present invention will typically be used with a nebulizer. The nebulizer can be either compressed air driven or ultrasonic. Any nebulizer known in the art can be used in conjunction with the present invention such as but not limited to: Ultravent, Mallinckrodt, Inc. (St. Louis, Mo.); the Acorn II nebulizer (Marquest Medical Products, Englewood Colo.). Other nebulizers useful in conjunction with the present invention are described in U.S. Pat. No. 4,624,251 issued Nov. 25, 1986; U.S. Pat. No. 3,703,173 issued Nov. 21, 1972; U.S. Pat. No. 3,561,444 issued Feb. 9, 1971 and U.S. Pat. No. 4,635,627 issued Jan. 13, 1971.

The formulation may include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable elimination half-time. Such macromolecules include but are not limited to soya lecithin, oleic acid, and sorbetan trioleate, with sorbitan trioleate preferred.

The formulations of the present embodiment may also include other agents useful for protein stabilization or for the regulation of osmotic pressure. Examples of the agents include but are not limited to salts, such as sodium chloride, or potassium chloride, and carbohydrates, such as glucose, galactose, or mannose, and the like.

J. Aerosol Dry Powder Formulations

It is also contemplated that the present pharmaceutical formulation will be used as a dry powder inhaler formulation comprising a finely divided powder form of the SABM and a dispersant. The form of the compound will generally be a lyophilized powder. Lyophilized forms of conjugate molecule can be obtained through standard techniques.

In another embodiment, the dry powder formulation will comprise a finely divided dry powder containing one or more compounds of the present invention, a dispersing agent and also a bulking agent. Bulking agents useful in conjunction with the present formulation include such agents as lactose, sorbitol, sucrose, or mannitol, in amounts that facilitate the dispersal of the powder from the device.

All publications (including patents and patent applications) cited herein are hereby incorporated in their entirety by reference.

Throughout this specification and claims, the word “comprise,” or variations such as “comprises” or “comprising,” will be understood to imply the inclusion of a stated integer or group of integers but not the exclusion of any other integer or group of integers.

The following examples are offered by way of illustration and not by way of limitation. The disclosures of all citations in the specification are expressly incorporated herein by reference.

### EXAMPLES

### Example 1

### Materials

For these studies, a Fab of a humanized antibody that binds to the extracellular domain of p185^{HER2} (HER2) was recombinantly engineered to include an albumin binding peptide (AB). The sequence of the variable region of the antibody used in this study, humAb4D5-8, can be found in Carter et al., (1992) PNAS 89:4285-4289. Previously,the humanized Fab had been derived from the murine monoclonal antibody muMAb4D5 (herein 4D5), which monoclonal antibody was produced by a hybridoma deposited with the American Type Culture Collection in Manassas, Va., and has ATCC accession number CRL 10463. Methods of making humanized anti-Her-2 antibodies and the identity of example variable domain sequences are provided in, e.g., U.S. Pat. Nos. 5,821,337 and 6,054,297 and in Carter et al., (1992) PNAS 89:4285-4289.

The nucleic acid sequence encoding the albumin binding peptide (“AB”), QRLMEDICLPRWGCLWEDDF (SEQ ID NO:1), was joined via a nucleic acid sequence encoding a linker sequence, GGGS (SEQ ID NO:422), to a nucleic acid sequence encoding the Fab. The nucleic acid sequence encoding the linker was joined to the heavy chain C-terminal KTHT residues of the Fab. As a control, an anti-tissue factor Fab containing the variable region of the D3H44 antibody fused to an AB through its light chain was constructed by recombinant DNA engineering. See Presta, L., et al., (2001) Thromb. Haemost. 85:379-389 for D3H44 amino acid sequence.

The resulting construct was expressed and secreted from E. coli as a fusion protein (“AB.Fab4D5-H” or “rhuABFabATFL”), then isolated and purified. Next, the fusion proteins were conjugated to monomethylauristatin (MMAE). For the tumor efficacy study, the fusion proteins were attached to MMAE via a valine-citrulline (val-cit or vc) dipeptide Linker reagent having a maleimide moiety and a para-aminobenzylcarbamoyl (PAB) spacer. See Klussman, K et al., (2004) Bioconjugate Chem. 15:765-773 for an example of methods for attaching MMAE to antibodies. For the toxicity study, the fusion proteins were attached to MMAE via, for example, conjugation with an MMAE modified with an activated derivative of maleimidocaproyl through their lysines using succinimidyl acetylthioacetate (Sata) to generate free thiols followed by conjugation to valine-citrulline-MMAE (“vc-MMAE”). The ratio of MMAE on the resulting AB.Fab4D5-H was generally an average around 1:1 with ratios as high as 4:1 and as low as 0:1 such that between 0-4 MMAE moieties were randomly distributed on exposed lysines, the overall average being about 1 MMAE per AB.Fab4D5-H.

### Example 2

### Efficacy Studies with MMAE Conjugates

AB.Fab-4D5-H-MMAE conjugates were tested against established MMTV-HER2 transgenic mammary tumors (Fo5). This tumor line is non-responsive to Herceptin® but responds well to a Herceptin®-MC-vc-PAB-MMAE conjugate.

A single intravenous dose of 1650 μg MMAE/m² rhuAB.Fab-4D5-H-vc-MMAE (i.e., with AB), rhuFab4D5vcMMAE (i.e., without AB), or rhuABFabATFL-vc-MMAE (negative control) was given to mMMTV-HER2 Fo5 tumor bearing mice. Each treated mouse had a mean tumor volume between 100 and 200 mm³. The MMAE-conjugated molecules or a phosphate buffer saline (“vehicle”) were administered on day 0 of the study and tumor measurements were performed twice weekly for 17 days. A known efficacious MMAE conjugate, Herceptin®-MC-vc-PAB-MMAE was run for comparison at a dose of 1245 μg/m2 MMAE. Log Cell Kill analyses based on tumor doubling times were conducted. The Log Cell Kill analysis uses a mathematical computation of tumor growth delay based on the time it takes for tumors to double in size after treatment begins compared to controls. The mathematical equation is:

### Tumor Doubling Time—Mean Doubling Time for Control

### 3.32× Mean Doubling Time for Control

FIG. 1 shows no significant difference between rhuAB.Fab4D5-H-vc-MMAE and the Herceptin®-MC-vc-PAB-MMAE group (p=0.0001) whereas the negative MMAE control Fab, rhuABFabATFL-vc-MMAE, was not significantly different from Vehicle control (p=0.6) by Fisher's PSLD.

### Example 3

### Toxicity of IgG-MMAE, F(ab′))-MMAE Conjugates and Free MMAE

Female Spraque-Dawley (SD) rats weighing between 75-80 grams (Charles River Laboratories, Hollister, Calif.) were used in the following studies to compare the toxicity of free MMAE, Fab-MMAE and F(ab′)₂-MMAE conjugates.

Dosing Groups:

| | | | μg | | Linkage | |
|---|---|---|---|---|---|---|
| Group | Administered | mg/kg | MMAE/m² | MMAE/MAb | site | #/sex |
| 1 | Vehicle (PBS) | 0 | 0 | 0 | NA | 6/F |
| 2 | Herceptin ®-val-cit-MMAE | 20.2 | 2105 | 5.3 | cysteine | 6/F |
| 3 | Herceptin ® F(ab′)2-val-cit-MMAE | 10.83 | 840 | 2.7 | lysine | 6/F |
| 4 | Herceptin ® F(ab′)2-val-cit-MMAE E | 27.14 | 2105 | 2.7 | lysine | 6/F |
| 5 | free MMAE | 0.516 | 2105 | | NA | 6/F |

Dosing Groups:

For the Herceptin®-val-cit-MMAE, the μg MMAE/m2 was calculated using 718 as the MW of MMAE and 145167 as the MW of Herceptin®. For the Herceptin® F(ab′)2-val-cit-MMAE, the μg MMAE/m2 was calculated using 718 as the MW of MMAE and 100000 as the MW of Herceptin® F(ab′)2. The body surface area was calculated as follows: [{(body weight in grams to 0.667 power)×11.8}/10000]. (Guidance for Industry and Reviewers. Estimating the Safety Starting Dose in Clinical Trials for Therapeutics in Adult Healthy Volunteers. U. S. Department of Health and Human Services Food and Drug Administration, Center for Drug Evaluation and Research (CDER), Center for Biologics Evaluation and research (CBER), December 2002).

The dose solutions were administered by a single intravenous bolus tail-vein injection on Study Day 1 at a dose volume of 10 mL/kg (diluted in PBS). General clinical observations were performed daily. Morbidity and mortality checks were performed twice daily (AM and PM). The body weights of the animals were measured pre-dose on Study Day 1 and daily thereafter. Whole blood was collected into EDTA containing tubes for hematology parameters (e.g., mean serum AST levels, ALT levels, GGT levels and billirubin levels) and differential cell counts (e.g., mean white blood cell counts and platlet counts). Whole blood was collected into serum separator tubes for clinical chemistry parameters. Blood samples were collected pre-dose on Study Day-4, on Study Day 3 and on Day 5 at necropsy. Whole blood was also collected into lithium heparin containing tubes at necropsy and the plasma was frozen at −70° C. for later analysis. The following tissues were collected at necropsy: liver, kidneys, heart, thymus, spleen, brain, sternum and sections of the GI tract, including stomach, large and small intestine. At necropsy, only spleen and thymus were weighed. All statistical analyses were done on Day 5 body weights using a One Way ANOVA, Tukey's test, (SigmaStat 2.03 Software).

On Study Day 3 animals on dose groups 4 and 5 (27.14 mg/kg Herceptin® F(ab′)2-val-cit-MMAE and 516 ug/kg free MMAE, respectively) were moribund. All group 4 and 5 animals were severely lethargic and most had a yellow discharge in the urogenital area. The animals in these dose groups were necropsied on Day 3. On study day 5 animals in dose group 3 (10.83 mg/kg Herceptin® F(ab′)2-val-cit-MMAE) also had yellow discharges in the urogenital area.

A complete set (baseline, days 3 & 5) of clinical chemistry and hematology data is only available for groups 1-3; day 5 data are not available for groups 4 & 5 as these animals were necropsied on day 3 due to significant morbidity or weight loss. This should also be considered in the interpretation of the histologic changes of these animals when comparing findings with those observed in animals terminated on day 5.

On day 3 animals of group 4 (high dose of the Herceptin® F(ab′)2 )and group 5 (free MMAE) showed the highest elevations in liver associated serum enzymes ALT, AST and GGT and the lowest platelet and white blood cell counts. The elevations in AST and ALT were similar in the two groups, however, group 4 showed significantly higher elevations in GGT and total bilirubin than group 5. Elevations in GGT and total bilirubin may indicate problems with the excretory liver function or the biliary system. The histologic evaluation did not show a morphologic correlate for this observation and it is unclear, whether differences in the PK characteristics of free MMAE vs immunoconjugate can account for this finding. The low dose Herceptin® F(ab′)2-vc-MMAE group 3 showed transient elevations of liver function tests on day 3, which returned to baseline levels by day 5. However, platelet and white blood cell counts remained decreased on day 5 without sign of recovery. Animals treated with the full length immunoconjugate Herceptin®-vc-MMAE (dose of MMAE matches groups 4 & 5) showed the typical toxicology profile of increased liver function tests and leuko- and thrombocytopenia. With the exception of bilirubin, all parameters showed progression over the five-day period; however, on day 3 changes were less severe in group 2 than in groups 4 and 5. Morphologically, the pattern of toxicity observed in groups 2-5 was identical and matched that seen in previous studies. Although only a limited number of organs was evaluated, clinical pathology data does not suggest significant organ-specific damage at other sites. The morphologic changes were least severe in group 3 (low dose Herceptin® F(ab′)2) and most severe in groups 4 and 5 (high dose Herceptin® F(ab′)2 and free MMAE). The changes included bone marrow hypocellularity, thymic atrophy with marked apoptotic activity, increased numbers of mitotic and apoptotic cells in intestinal mucosa with variable extent of mucosal degeneration and atrophy and increased numbers of mitotic and apoptotic cells among hepatocytes and biliary epithelium. Animals of groups 2, 4 and 5 also showed evidence of hepatocyte dropout and occasional areas of hepatic necrosis.

Animals in dose groups 4 and 5 (27.14 mg/kg Herceptin® F(ab′)2-val-cit-MMAE and 516 ug/kg free MMAE, respectively) lost 14.5 and 12 grams body weight, respectively, by Day 3 compared with Day 1 weights. The decrease in body weight in Group 2 animals administered a comparable amount of MMAE (2105 ug MMAE/m2) was not as severe as in group 4 and 5 animals. As shown in FIG. 2, the regimen of free MMAE resulted in a similar weight loss profile as the Fa(b′)₂ regimen (without a serum albumn binding protein). Weight loss is an indicator of toxicity.

In sum, Herceptin® F(ab′)2-val-cit-MMAE (lysine) caused acute toxicity in a dose-dependent fashion. Animals in the high dose Herceptin® F(ab′)2-val-cit-MMAE showed a significantly greater weight loss compared with animals receiving the same amount of drug as Herceptin®-val-cit-MMAE. Animals administered free MMAE at a comparable dose (2150 ug/m2) to the high dose of Herceptin® F(ab′)2-val-cit-MMAE had comparable weight loss and changes in liver associated serum enzyme levels and white blood cell and platelet counts. The findings are consistent with the administration of agents that inhibit tubulin formation (Wood K W, Cornwell W D, and Jackson J R. Past and future of the mitotic spindle as an oncology target. Current Opinion in Pharmacology, 1:370-377. 2001).

### Example 4

### Toxicity Studies with MMAE-Fab Conjugates

The toxicity of Herceptin®-monomethylauristatin (MMAE) immunoconjugates, Fab4D5-MMAE and AB.Fab4D5-H-MMAE immunoconjugates were compared in female Sprague-Dawley rats (80-100 grams).

Female rats were administered equivalent doses (2105 ug MMAE/m²) via tail-vein injections at a dose volume of 10 ml/kg (diluted in PBS). All dose solutions were administered as a single bolus injection.

Dosing Groups:
1=PBS, 6 females
2=20.2 mg/kg Herceptin®-val-cit-MMAE, 6 females
3=5.7 mg/kg Fab4D5-vc-MMAE
4=14.24 mg/kg Fab4D5-vc-MMAE, 6 females
5=7.85 mg/kg AB.Fab4D5-H-vc-MMAE
6=19.62 mg/kg AB.Fab4D5-H-vc-MMAE, 6 females

In a previous study, the 2105 ug MMAE/m² dose of Herceptin®-val-cit-MMAE resulted in changes in liver associated serum enzyme levels and hematology parameters that were moderately severe. Doses of 5.7 mg/kg rhuFab4D5-val-cit-MMAE and 7.85 mg/kg rhuFab4D5-H-val-cit-MMAE were also administered in the present study to give an MMAE exposure of approximately 840 ug MMAE/m².

In these assays, blood samples (approximately 500 ul) were generally collected via the retro-orbital sinus under isofluorane anesthesia on Study Days-3 (pre-dose) and-Day 3 for clinical chemistry and hematology. Blood was also collected on Day 5 at necropsy via the inferior vena cava under ketamine anesthesia. Clinical observations and body weight recordings were performed once daily and cageside mortality checks were conducted twice daily (am/pm). Animals which were moribund were euthanized.

During necropsy on Study Day 5, the blood of the rats were collected via the abdominal aorta for clinical chemistry and hematology. The following tissues were collected: heart, lung, trachea, liver, kidney, thymus, spleen, brain, axillary lymph nodes, entire gastrointestinal tract, skin, urinary bladder, and bone marrow. Additionally, organ weights will be recorded for liver, thymus, spleen, and brain. At necropsy, the liver, spleen and thymus were weighed. The tests included the test for group mean change in animal body weight, white blood cell count, platelet counts, AST levels, ALT levels, GGT levels, and serum Billirubin levels.

On Study Day 4 animals in dose groups 3 and 4 (5.7 and 14.25 mg/kg rhuFab4D5-val-cit-MMAE) had moderate pilo-erection and many of the animals were lethargic. Two animals in the 14.25 mg/kg rhuFab4D5-val-cit-MMAE dose group were moribund on day 4 and were necropsied.

The auristatin E conjugated full length antibody (Herceptin®-MC-val-cit-PAB-MMAE, group 2) showed the same toxicity profile as seen in previous studies: Liver function tests were elevated on days 3 and 5 and showed, with the exception of GGT, evidence of recovery by day 5. The same animals showed progressive neutro- and thrombocytopenia during the 5-day study. Animals treated with the two types of antibody fragments (rhuFab4D5 and rhuFab 4D5-H ) showed dose-dependent toxicity. The liver associated serum enzyme levels on days 3 and 5 are essentially identical to vehicle-treated animals for groups 3 and 5 (low doses of rhuFab 4D5 and rhuFab 4D5-H, respectively). There was a very mild elevation of AST and ALT in animals of group 5 (rhuFab4D5-H-val-cit-MMAE), however, whether this change is statistically significant and whether it actually represents hepatotoxicity is unclear. Animals in group 3 (low dose rhuFab4D5-val-cit-MMAE ), showed a very mild thrombocytopenia and a 50% decrease of leukocytes on day 5, whereas animals in group 5 showed normal leukocyte counts on day 5 (after a mild transitory decline on day 3) and a mild thrombocytosis on day 5. Animals in groups 4 and 6 (high doses of rhuFab 4D5 and rhuFab 4D5-H, respectively) showed clear signs of toxicity on days 3 and 5. Toxicity appeared more severe in group 4, two animals were euthanized on day 4 based on signs of morbidity. Levels of AST, ALT and GGT are higher in animals of group 4 than group 2 or 6 (comparable drug doses of Herceptin®-val-cit-MMAE and rhuFab4D5-H-val-cit-MMAE), at both time points. The levels are slightly lower in animals of group 6 than group 2. Animals in group 4 and 6 showed profound thrombo- and leukocytopenia on day 5. The levels are lower than those seen in animals of group 2 and animals in group 6 seem to do slightly better than animals in group 4.

The results of the histopathological evaluation correlate very well with the clinical observations (body weight measurements) and the clinical pathology data. Animals in groups 2, 3, 4 and 6 show a markedly hypocellular bone marrow; signs of regeneration are only present in animals of group 3. In contrast, animals in group 5 show bone marrows of near normal cellularity. Similar findings are observed in the thymus: Animals of groups 2, 4 and 6 show marked atrophy and apoptotic activity, whereas animals of groups 3 and 5 show mild atrophy without significant apoptotic activity. The changes in liver, small and large intestine are more difficult to quantify, however, the number of mitotic and/or apoptotic cells in these organs appears greater in animals of groups 2, 4 and 6 than those in groups 3 and 5. Small areas of necrosis are only observed in two animals of group 4. Interestingly, only animals in group 5 (in addition to vehicle-treated animals) retain small foci of extramedullary hematopoiesis in the liver suggesting lower levels of free drug in these animals. Clinical pathology or histopathology data do not show any evidence of a different pattern of toxicity in animals treated with Fab immunoconjugates compared with animals treated with full length antibody conjugate.

Animals treated with the two types of antibody fragments showed dose-dependent toxicity. Animals administered high doses of rhuFab 4D5-val-cit-MMAE and rhuFab 4D5-H-val-cit-MMAE (containing albumin binding peptide) showed clear signs of toxicity on days 3 and 5. Toxicity appeared more severe in the rhuFab 4D5 group, two animals were euthanized on day 4 because they were moribund. The results of the histopathological evaluation correlate very well with the clinical observations (body weight measurements) and the clinical pathology data. Clinical pathology or histopathology data did not show any evidence of a different pattern of toxicity in animals treated with Fab immunoconjugates compared with animals treated with full length antibody conjugate. The findings are consistent with the administration of agents that inhibit tubulin formation (Wood et al, 2001).

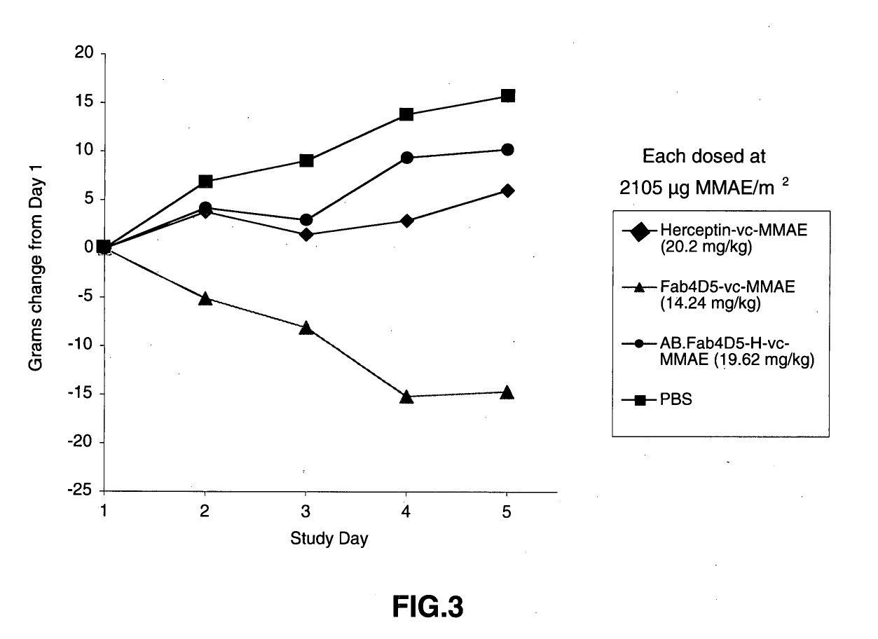

FIG. 3 indicates that the albumin binding peptide can alter the toxicity of a drug conjugate. Ab.Fab4D5-H-vc-MMAE (containing the albumin binding peptide) was significantly less toxic in rats than Fab4D5-vc-MMAE at Study Day 5. The group average change in body weight in animals administered 5.7 mg/kg rhuFab4D5-val-cit-MMAE and 7.85 mglkg rhuFab4D5-H-val-cit-MMAE (Groups 3 and 5) were not significantly different from each other. Dose groups 2, 4, and 6 (20.2 mg/kg Herceptin®-val-cit-MMAE, 14.24 mg/kg rhuFab4D5-val-cit-MMAE, and 19.62 mg/kg rhuFab4D5-H-val-cit-MMAE, respectively) all received 2105 ug/M2 MMAE. The group average decrease in body weight in Group 2 and 4 animals was more severe than group 6 animals.

### Example 5

### Affinity Measurements by Surface Plasmon Resonance

Binding affinities between SA peptides and album were obtained using a BIAcore 3000 (BIAcore, Inc., Piscataway, N.J.). Albumin was captured in a CM5 chip using amine coupling at approximately 5000 resonance units (RU). SA peptides (0, 0.625, 1.25, 2.5, 5, and 10 μM were injected at a flow rate of 20 μl/minute for 30 seconds. The bound peptides were allowed to disassociate for 5 minutes before matrix regeneration using 10 mM glycine, pH 3.

The signal from an injection passing over an uncoupled cell was subtracted from that of an immobilized cell to generate sensongrams corresponding to the amount of peptide bound as a function of time. The running buffer, PBS containing 0.05% TWEEN-20T, was used for all sample dilutions. BIAcore kinetic evaluation software (v 3.1) was used to determine the dissociation constant (K_{d}) from the association and dissociation rates, using a one to one binding model.

The affinity of selected peptides for binding human (HAS), rabbit (BuSA), rat (RSA), and mouse (MSA) albumin was assessed by the BlAcore assay as well as SA08 peptide competition assay. The data, shown below in Table 8, demonstrate that the IC₅₀ values obtained in the competition assay compared favorably with the Kd values obtained in the BlAcore assay. Peptide SA15, representing the consensus peptide for binding rabbit albumin, had the lowest IC₅₀ value in the competition assay and the highest affinity by surface plasmon resonance for rabbit albumin. A linear peptide, identical to SA06, but having both Cys residues altered to Ala, had an IC₅₀ that was greater than 50 μM, demonstrating the importance of the disulfide.

### Example 6

### Determination of Relative Kd

Introduction:

In assessing the binding capacity between proteins, ELISA has been the method of choice. The ease of developing a highly specific and quantitative assay has resulted in ELISA wide application. However, in the format where protein is immobilized directly on the solid surface, the potential artifact due to denaturing or obscuring binding epitope can occur.

To determine the affinity of albumin binding peptide conjugated to Fab molecules (Fab-H) to Albumin, we developed two types of ELISA. The first format involved the adsorption of albumin to the well surface and the bound Fab is detected with goat-anti-huFab-HRP. The second format involves the binding of Fab-H with albumin in solution and determines the dissociation constant (Kd). The basic principle of the second assay is to allow the binding, of a constant concentration of Fab-H to varying amount of albumin, to reach equilibrium in solution, and determine the un-bound Fab-H in ELISA well coated with albumin. The Kd value can be determined by analyzing the data using Scatchard Analysis (Munson et al., 1980, Anal. Biochem., 107: 220)

Materials & Methods:

Material:

Mouse Albumin—Lyophilized form, Cat. No. A3139

Rat Albumin—Lyophilized form, Cat. No. A6414

Rabbit Albumin—Lyophilized form, Cat. No. A0639

1 mg/ml Albumin solution was prepared by dissolving 10 mg in 10 ml of PBS. The solution is stored at 4° C.

Assay Buffer: PBS+0.5% Chicken Egg Albumin (Sigma #A5503)+)0.5% Tween 20, PH 7.4)

Direct Binding ELISA Assay

Mouse, Rat, or Rabbit albumin (Sigma) was immobilized onto NUNC Maxisorp 96-well plates at 2 μg/ml overnight at 4° C. After removal of the coating solution, the plates were blocked with binding buffer (PBS, 0.5% ovalbumin and 0.05% Tween 20) for 1 hour at 25° C. Serially diluted Fab-Hx in binding buffer, were added at 100 ul per well and allowed to bind to coated albumin for 30 minutes at 25° C. The unbound Fab-Hx was removed by washing the well with 0.05% PBS/Tween20 and the bound Fab-Hx molecules were detected by I hour incubation with Goat anti-human Fab′2-HRP for at 25° C. Bound HRP was then measured with a solution of tetramethylbenzidine (TMB)/H₂0₂. After 15 minutes incubation, the reaction was quenched by the addition of 1M phosphoric acid. The absorbance at 450 nm was read with a reference wavelength of 650 nm.

Kd (Solution Binding with Preincubation) ELISA Assay

A fixed concentration of Fab-H (determined in above binding ELISA) was first incubated in solution with varying concentrations of albumin in Assay Buffer. After ≧2 hours of incubation at room temperature, 100 μl of the mixture was transferred to Albumin coated ELISA plates. The concentration of free Fab-Hx was then determined by the direct binding ELISA as described above.

The fixed concentration of Fab-H and the starting concentration of albumin are listed in the following table. Albumin were 1:3 serially diluted for 8 points.

| Molecules | Fab-H | | Fab-H4 | | Fab-H8 | | Fab-H10 | | Fab-H11 | |
|---|---|---|---|---|---|---|---|---|---|---|
| [Fab-H] | 0.5 | nM | 200 | nM | 22.5 | nM | 3 | μM | 3 | μM |
| [Rabbit | 3 | μM | 3 | μM | 3 | μM | 30 | μM | 30 | μM |
| Albumin] | | | | | | | | | | |
| [Fab-H] | 0.25 | nM | 0.125 | nM | 0.125 | nM | 12 | nM | 800 | nM |
| [Rat | 3 | μM | 3 | μM | 3 | μM | 30 | μM | 60 | μM |
| Albumin] | | | | | | | | | | |
| [Fab-H] | 0.25 | nM | 6.25 | pM | 31.25 | pM | 62.5 | nM | 62.5 | nM |
| [Mouse | 3 | μM | 3 | μM | 3 | μM | 119 | μM | 119 | μM |
| Albumin] | | | | | | | | | | |

Results:

The affinity measurement using ELISA was first published by Friguet et. al. in 1985. We have used this methodology in determining the Kd and selecting humanized antibody to HER2 ECD. (Carter et. Al., Proc. Natl. Acad. Sci. 89, 4285, 1992 ).

In general, binding equilibrium studies require that the concentration of antibody should be close to, or lower than, the value of the dissociation constant. Since the dissociation constant is a priori unknown, it is therefore best to choose total Fab-H concentration that will give sufficient absorbance in the binding ELISA used to measure the free Fab-Hx. This concentration was determined by titrating Fab-Hx in the direct binding ELISA.

To verify that the antibody-albumin had reached equilibrium, Fab-H was incubated with rabbit albumin at 1 hr., 2 hr. and overnight, and then the reaction mixtures were assayed in the binding ELISA. Equilibrium was reached after 2 hours incubation.

To determine the optimal time needed for free Fab-H to bind to coated albumin in the well, the Ab-albumin mixture was incubated with coated albumin in the well for 15, 30, 45, 60 and 120 minute. Based on this assay, it was determined that 30 minutes was the minimum amount of time required to bind all the free Fab-H. The results of the Kd (solution binding with preincubation) ELISA assay as shown in Table 10.

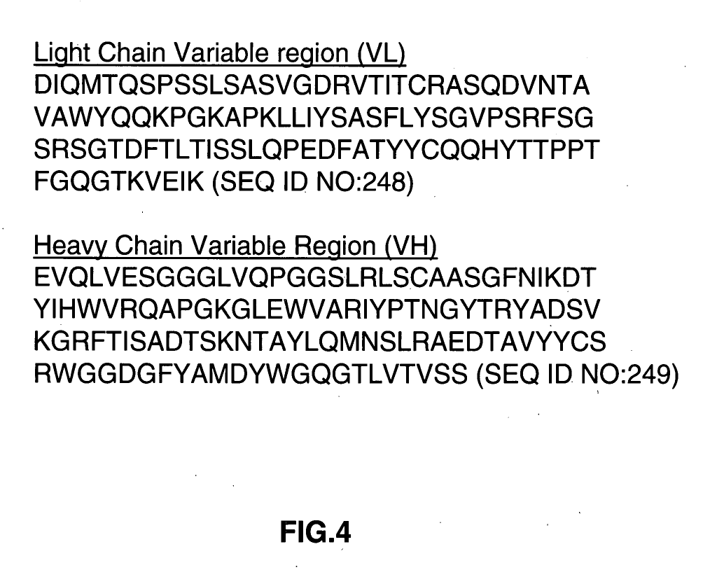

LISTING OF SEQUENCES

| | |
|---|---|
| SEQ | |
| ID | |
| No. | SEQUENCE |

| | |
|---|---|
| 1 | Phe-Cys-Xaa-Asp-Trp-Pro-Xaa-Xaa-Xaa-Ser-Cys |
| 2 | Val-Cys-Tyr-Xaa-Xaa-Xaa-Ile-Cys-Phe |
| 3 | Cys-Tyr-Xaa₁-Pro-Gly-Xaa-Cys |
| 4 | Asp-Xaa-Cys-Leu-Pro-Xaa-Trp-Gly-Cys-Leu-Trp |
| 5 | Trp-Cys-Asp-Xaa-Xaa-Leu-Xaa-Ala-Xaa-Asp-Leu-Cys |
| 6 | Asp-Leu-Val-Xaa-Leu-Gly-Leu-Glu-Cys-Trp |
| 7 | DLCLRDWGCLW |
| 8 | DICLPRWGCLW |
| 9 | MEDICLPRWGCLWGD |
| 10 | QRLMEDICLPRWGCLWEDDE |
| 11 | QGLIGDICLPRWGCLWGDSV |
| 12 | QGLIGDICLPRWGCLWGDSVK |
| 13 | EDICLPRWGCLWEDD |
| 14 | RLMEDICLPRWGCLWEDD |
| 15 | MEDICLPRWGCLWEDD |
| 16 | MEDICLPRWGCLWED |
| 17 | RLMEDICLARWGCLWEDD |
| 18 | EVRSFCTRWPAEKSCKPLRG |
| 19 | RAPESFVCYWETICFERSEQ |
| 20 | EMCYFPGICWM |
| 21 | CXXGPXXXXC |
| 22 | XXXXCXXGPXXXXCXXXX |
| 23 | CXXXXXXCXXXXXXCCXXXCXXXXXXC |
| 24 | CCXXXCXXXXXXC |
| 25 | CCXXXXXCXXXXCXXXXCC |
| 26 | CXCXXXXXXXCXXXCXXXXXX |
| 27 | GENWCDSTLMAYDLCGQVNM |
| 28 | MDELAFYCGIWECLMHQEQK |
| 29 | DLCDVDFCWF |
| 30 | KSCSELHWLLVEECLF |
| 31 | EVRSFCTDWPAEKSCKPLRG |
| 32 | CEVALDACRGGESGCCRHICELIRQLC |
| 33 | RNEDPCVVLLEMGLECWEGV |
| 34 | DTCVDLVRLGLECWG |
| 35 | QRQMVDFCLPQWGCLWGDGF |
| 36 | CGCVDVSDWDCWSECLWSHGA |
| 37 | GEDWCDSTLLAFDLCGEGAR |
| 38 | GENWCDWVLLAYDLCGEDNT |
| 39 | MELWCDSTLMAYDLCGDFNM |
| 40 | EVRSFCTDWPAHYSCTSLQG |
| 41 | GRSFCMDWPAHKSCTPLML |
| 42 | GVRTFCQDWPAHNSCKLLRG |
| 43 | QTRSFCADWPRHESCKPLRG |
| 44 | RRTCDWPHNSCKLRG |
| 45 | RAAESSVCYWPGICFDRTEQ |
| 46 | MEPSRSVCYAEGICFDRGEQ |
| 47 | REPASLVCYFEDICFVRAEA |
| 48 | RGPDVCYWPSICFERSMP |
| 49 | LVPERIVCYFESICYERSEL |
| 50 | RMPASLPCYWETICYESSEQ |
| 51 | RTAESLVCYWPGICFAQSER |
| 52 | RAPERWVCYWEGICFDRYEQ |
| 53 | EICYFPGICWI |
| 54 | ELCYFPGICWT |
| 55 | DICYIPGICWM |
| 56 | KLCYFPGICWS |
| 57 | DLCYFPGICWM |
| 58 | GMCYFPGICWA |
| 59 | EMCYFPGICWS |
| 60 | EMCYFPGICWT |
| 61 | KTCYFPGICWM |
| 62 | KVCYFPGICWM |
| 63 | DVCYFPGICWM |
| 64 | EICYFPGICWM |
| 65 | ALCYFPGICWM |
| 66 | ELCYFPGICWP |
| 67 | ELCYFPGICWM |
| 68 | DMCYFPGICWL |
| 69 | DMCYFPGICFN |
| 70 | ETCYFPGICWL |
| 71 | EVCYFPGICWF |
| 72 | EVCYFPGICWE |
| 73 | EVCYFPGICWM |
| 74 | LAEMCYFPGICWMSA |
| 75 | GGEICYFPGICRVLP |
| 76 | EHDMCYFPGICWIAD |
| 77 | VQEVCYFPGICWMQE |
| 78 | SREVCYYPGICWNGA |
| 79 | DSEVCYFPGICWSGT |
| 80 | GTEVCYFPGICWGGG |
| 81 | SYAPCYFPGICWMGN |
| 82 | HAEICYFPGICWTER |
| 83 | NDEICYFPGVCWKSG |
| 84 | RDTVCYFPGICWMAS |
| 85 | VRDMCYFPGICWKSE |
| 86 | ASEICYFPGICWMVE |
| 87 | QTELCYFPGICWNES |
| 88 | TTEMCYFPGICWKTE |
| 89 | KTEICYFPGICWMSG |
| 90 | QCFPGWVK |
| 91 | IVEMCYYPGICWISP |
| 92 | SGAICYVPGICWTHA |
| 93 | QRHPEDICLPRWGCLWGDDD |
| 94 | NRQMEDICLPQWGCLWGDDF |
| 95 | QRLMEDICLPRWGCLWGDRF |
| 96 | QWHMEDICLPQWGCLWGDVL |
| 97 | QWQMENVCLPKWGCLWEELD |
| 98 | LWAMEDICLPKWGCLWEDDF |
| 99 | LRLMDNICLPRWGCLWDDGF |
| 100 | HSQMEDICLPRWGCLWGDEL |
| 101 | QWQVMDICLPRWGCLWADEY |
| 102 | HRLVEDICLPRWGCLWGNDF |
| 103 | QMHMMDICLPKWGCLWGDTS |
| 104 | LRIFEDICLPKWGCLWGEGF |
| 105 | QSYMEDICLPRWGCLSDDAS |
| 106 | QGDFWDICLPRWGCLSGEGY |
| 107 | RWQTEDVCLPKWGCLFGDGV |
| 108 | LIFMEDVCLPQWGCLWEDGV |
| 109 | QRDMGDICLPRWGCLWEDGV |
| 110 | QRHMMDFCLPKWGCLWGDGY |
| 111 | QRPIMDFCLPKWGCLWEDGF |
| 112 | ERQMVDFCLPKWGCLWGDGF |
| 113 | QGYMVDFCLPRWGCLWGDAN |
| 114 | KMGRVDFCLPKWGCLWGDEL |
| 115 | QSQLEDFCLPKWGCLWGDGF |
| 116 | QGGMGDFCLPQWGCLWGEDL |
| 117 | QRLMWEICLPLWGCLWGDGL |
| 118 | QRQIMDFCLPHWGCLWGDGF |
| 119 | GRQVVDFCLPKWGCLWEEGL |
| 120 | QMQMSDFCLPQWGCLWGDGY |
| 121 | KSRMGDFCLPEWGCLWGDEL |
| 122 | ERQMEDFCLPQWGCLWGDGV |
| 123 | QRQVVDFCLPQWGCLWGDGS |
| 124 | DICLPEWGCLW |
| 125 | DICLPVWGCLW |
| 126 | DLCLPEWGCLW |
| 127 | DLCLPKWGCLW |
| 128 | DLCLPVWGCLW |
| 129 | DICLPAWGCLW |
| 130 | DICLPDWGCLW |
| 131 | DICLERWGCLW |
| 132 | EWDVCLPHWGCLWDG |
| 133 | WDDICFRDWGCLWGS |
| 134 | MDDICLHHWGCLWDE |
| 135 | MDDLCLPNWGCLWGD |
| 136 | FEDFCLPNWGCLWGS |
| 137 | FEDLCVVRWGCLWGD |
| 138 | WEDLCLPDWGCLWED |
| 139 | SEDFCLPVWGCLWED |
| 140 | DFDLCLPDWGCLWDD |
| 141 | NWDLCFPDWGCLWDD |
| 142 | EEDLCLPVWGCLWGA |
| 143 | EEDVCLPVWGCLWEG |
| 144 | MFDLCLPKWGCLWGN |
| 145 | EFDLCLPTWGCLWED |
| 146 | MWDVCFPDWGCLWDV |
| 147 | EWDVCFPAWGCLWDQ |
| 148 | VWDLCLPQWGCLWDE |
| 149 | DTCADLVRLGLECWA |
| 150 | NTCADLVRLGLECWA |
| 151 | DTCDDLVQLGLECWA |
| 152 | DTCEDLVRLGLECWA |
| 153 | DSCGDLLRLGLECWA |
| 154 | DTCSDLVGLGLECWA |
| 155 | X₅DXCLPXWGCLWX₄ |
| 156 | X₄DXCLPXWGCLWX₃ |
| 157 | AAQVGDICLPRWGCLWSEYA |
| 158 | AGWAADVCLPRWGCLWEEDV |
| 159 | ASVVDDICLPVWGCLWGEDI |
| 160 | ATMEDDICLPRWGCLWGAEE |
| 161 | DEDFEDYCLPPWGCLWGSSM |
| 162 | EGTWDDFCLPRWGCLWLGER |
| 163 | ERWEGDVCLPRWGCLWGESG |
| 164 | GDWMHDICLPKWGCLWDEKA |
| 165 | GIEWGDTCLPKWGCLWRVEG |
| 166 | GQQGEDVCLPVWGCLWDTSS |
| 167 | GRYPMDLCLPRWGCLWEDSA |
| 168 | GSAGDDLCLPRWGCLWERGA |
| 169 | HASDWDVCLPGWGCLWEEDD |
| 170 | LGVTHDTCLPRWGCLWDEVG |
| 171 | LVWEEDFCLPKWGCLWGAED |
| 172 | NVGWNDICLPRWGCLWAQES |
| 173 | QGVEWDVCLPQWGCLWTREV |
| 174 | RLDAWDICLPQWGCLWFEPS |
| 175 | SEAPGDYCLPRWGCLWAQEK |
| 176 | TAMDEDVCLPRWGCLWGSGS |
| 177 | TEIGQDFCLPRWGCLWVPGT |
| 178 | TLGWPDFCLPKWGCLWRESD |
| 179 | TLSNQDICLPGWGCLWGGIN |
| 180 | TSTGGDLCLPRWGCLWDSSE |
| 181 | VSEMDDICLPLWGCLWADAP |
| 182 | VSEWEDICLPSWGCLWETQD |
| 183 | VVGDGDFCLPKWGCLWDQAR |
| 184 | VVWDDDVCLPRWGCLWEEYG |
| 185 | WSDSDDVCLPRWGCLWGNVA |
| 186 | WVEEGDICLPRWGCLWESVE |
| 187 | AQAMGDICLPRWGCLWEAEI |
| 188 | ASDRGDLCLPYWGCLWGPDG |
| 189 | ASDPGDVCLPRWGCLWGESF |
| 190 | ASNWEDVCLPRWGCLWGERN |
| 191 | ASTPRDICLPRWGCLWSEDA |
| 192 | DGEEGDLCLPRWGCLWALEH |
| 193 | EGEEVDICLPQWGCLWGYPV |
| 194 | EVGDLDLCLPRWGCLWGNDK |
| 195 | FRDGEDFCLPQWGCLWADTS |
| 196 | GDMVNDFCLPRWGCLWGSEN |
| 197 | GRMGTDLCLPRWGCLWGEVE |
| 198 | HEWERDICLPRWGCLWRDGD |
| 199 | KKVSGDICLPIWGCLWDNDY |
| 200 | LLESDDICLPRWGCLWHEDG |
| 201 | MQAESDFCLPHWGCLWDEGT |
| 202 | MQGPLDICLPRWGCLWGGVD |
| 203 | QMPLEDICLPRWGCLWEGRE |
| 204 | REEWGDLCLPTWGCLWETKK |
| 205 | RVWTEDVCLPRWGCLWSEGN |
| 206 | SIREYDVCLPKWGCLWEPSA |
| 207 | SPTEWDMCLPKWGCLWGDAL |
| 208 | SSGLEDICLPNWGCLWADGS |
| 209 | SVGWGDICLPVWGCLWGEGG |
| 210 | TEENWDLCLPRWGCLWGDDW |
| 211 | TSGSDDICLPVWGCLWGEDS |
| 212 | TWPGDLCLPRWGCLWEAES |
| 213 | WDHELDFCLPVWGCLWAEDV |
| 214 | WTESEDICLPGWGCLWGPEV |
| 215 | WVPFEDVCLPRWGCLWSSYQ |
| 216 | EEDSDICLPRWGCLWNTS |
| 217 | EGYWDLCLPRWGCLWELE |
| 218 | ELGEDLCLPRWGCLWGSE |
| 219 | ETWSDVCLPRWGCLWGAS |
| 220 | GDYVDLCLPGWGCLWEDG |
| 221 | GVLDDICLPRWGCLWGPK |
| 222 | HMMDDVCLPGWGCLWASE |
| 223 | IDYTDLCLPAWGCLWELE |
| 224 | IEHEDLCLPRWGCLWAVD |
| 225 | ISEWDLCLPRWGCLWDRS |
| 226 | ISWADVCLPKWGCLWGKD |
| 227 | ISWGDLCLPRWGCLWEGS |
| 228 | KLWDDICLPRWGCLWSPL |
| 229 | LAWPDVCLPRWGCLWGGM |
| 230 | LNESDICLPTWGCLWGVD |
| 231 | LPEQDVCLPVWGCLWDAN |
| 232 | MAWGDVCLPRWGCLWAGG |
| 233 | NEEWDVCLPRWGCLWGGV |
| 234 | QELQDFCLPRWGCLWGVG |
| 235 | QREWDVCLPRWGCLWSDV |
| 236 | QRFWDTCLPRWGCLWGGD |
| 237 | RVFTDVCLPRWGCLWDLG |
| 238 | SGWDDVCLPVWGCLWGPS |
| 239 | SSASDYCLPRWGCLWGDL |
| 240 | SWQGDICLPRWGCLWGVD |
| 241 | SYETDVCLPYWGCLWEDA |
| 242 | SYWGDVCLPRWGCLWSEA |
| 243 | TLEWDMCLPRWGCLWTEQ |
| 244 | VGEFDICLPRWGCLWDAE |
| 245 | VTSWDVCLPRWGCLWEED |
| 246 | WLWEDLCLPKWGCLWEED |
| 247 | ALFEDVCLPVWGCLWGGE |
| 248 | ASEWDVCLPTWGCLWMEG |
| 249 | AYSADICLPRWGCLWMSE |
| 250 | EDWEDICLPQWGCLWEGM |
| 251 | EDWTDLCLPAWGCLWDTE |
| 252 | EEWEDLCLPRWGCLWSAE |
| 253 | EFWQDICLPNWGCLWAES |
| 254 | EGFSDICLPRWGCLWSQE |
| 255 | ETWEDLCLPNWGCLWDLE |
| 256 | GEVNDFCLPRWGCLWEGD |
| 257 | GGEWDVCLPAWGCLWGEE |
| 258 | KDWYDICLPRWGCLWGGE |
| 259 | KLGQDICLPRWGCLWDFA |
| 260 | LEEWDICLPQWGCLWREG |
| 261 | LVLPDICLPKWGCLWGDT |
| 262 | MDLADICLPKWGCLWESD |
| 263 | MVLDDICLPRWGCLWSEK |
| 264 | MWSGDLCLPRWGCLWGET |
| 265 | NRMGDICLPRWGCLWDGH |
| 266 | RDWEDLCLPNWGCLWELS |
| 267 | RGDWDLCLPKWGCLWEGV |
| 268 | RQWEDICLPRWGCLWGVG |
| 269 | RVEYDLCLPRWGCLWEPP |
| 270 | SIWSDICLPRWGCLWESD |
| 271 | TDEWDICLPNWGCLWEAG |
| 272 | TEDVDFCLPLWGCLWEEP |
| 273 | VKEEDFCLPRWGCLWEAG |
| 274 | WDFEDICLPRWGCLWADM |
| 275 | WEDWDVCLPRWGCLWGGG |
| 276 | YEDIDICLPRWGCLWDLS |
| 277 | AGLDEDICLPRWGCLWGKEA |
| 278 | AGMMGDICLPRWGCLWQGEP |
| 279 | APGDWDFCLPKWGCLWDDDA |
| 280 | AQLFDDICLPRWGCLWSDGY |
| 281 | ARTMGDICLPRWGCLWGASD |
| 282 | AWQDFDVCLPRWGCLWEPES |
| 283 | DTTWGDICLPRWGCLWSEEA |
| 284 | EGFLGDICLPRWGCLWGHQA |
| 285 | EQWLHDICLPKWGCLWDDTD |
| 286 | ETGWPDICLPRWGCLWEEGE |
| 287 | FELGEDICLPRWGCLWEEHN |
| 288 | GASLGDICLPRWGCLWGPED |
| 289 | GEWWEDICLPRWGCLWGSSS |
| 290 | GSLESDICLPRWGCLWGIDE |
| 291 | GWLEEDICLPKWGCLWGADN |
| 292 | HEQWDDICLPRWGCLWGGSY |
| 293 | QRVDDDICLPRWGCLWGENS |
| 294 | SVGWGDICLPKWGCLWAESD |
| 295 | TLMSNDICLPRWGCLWDEPK |
| 296 | TLVLDDICLPRWGCLWDMTD |
| 297 | TWQGEDICLPRWGCLWDTEV |
| 298 | VGVFDDICLPRWGCLWEQPV |
| 299 | VPAMGDICLPRWGCLWEARN |
| 300 | VSLGDDICLPKWGCLWEPEA |
| 301 | VWIDRDICLPRWGCLWDTEN |
| 302 | WRWNEDICLPRWGCLWEEEA |
| 303 | AVSWADICLPRWGCLWERAD |
| 304 | AWLDEDICLPKWGCLWNTGV |
| 305 | FSLDEDICLPKWGCLWGAEK |
| 306 | GDLGDDICLPRWGCLWDEYP |
| 307 | GEGWSDICLPRWGCLWAEDE |
| 308 | GLMGEDICLPRWGCLWKGDI |
| 309 | GWHDRDICLPRWGCLWEQND |
| 310 | LLGGHDICLPRWGCLWGGDV |
| 311 | MRWSSDICLPKWGCLWGDEE |
| 312 | QFEWDDICLPRWGCLWEVEV |
| 313 | QGWWHDICLPRWGCLWEEGE |
| 314 | REGWPDICLPRWGCLWSETG |
| 315 | RELWGDICLPRWGCLWEHAT |
| 316 | RLELMDICLPRWGCLWDPQD |
| 317 | SGVLGDICLPRWGCLWEEAG |
| 318 | SLGLTDLCLPRWGCLWEEEQ |
| 319 | SSLEQDICLPRWGCLWGQDA |
| 320 | SVLSDDICLPRWGCLWWDFS |
| 321 | TSLLDDICLPRWGCLWYEEG |
| 322 | TSLADDICLPRWGCLWSEDG |
| 323 | VEMWHDICLPRWGCLWDSNA |
| 324 | WDLASDICLPRWGCLWEEEA |
| 325 | FITQDICLPRWGCLWGEN |
| 326 | FLWRDICLPRWGCLWSEG |
| 327 | FVHEDICLPRWGCLWGEG |
| 328 | GLGDDICLPRWGCLWGRD |
| 329 | GMFDDICLPKWGCLWGLG |
| 330 | GPGWDICLPRWGCLWGEE |
| 331 | GPWYDICLPRWGCLWDGV |
| 332 | GWDDDICLPRWGCLWGDG |
| 333 | LEYEDICLPKWGCLWGGE |
| 334 | LLDEDICLPRWGCLWGVR |
| 335 | LMSPDICLPKWGCLWEGD |
| 336 | LVLGDICLPRWGCLWESD |
| 337 | MLSRDICLPRWGCLWEEE |
| 338 | MPWTDICLPRWGCLWSES |
| 339 | RLGSDICLPRWGCLWGAG |
| 340 | RLGSDICLPRWGCLWDYQ |
| 341 | SPWMDICLPRWGCLWESG |
| 342 | STFTDICLPRWGCLWELE |
| 343 | SVLSDICLPRWGCLWEES |
| 344 | TWFSDICLPRWGCLWEPG |
| 345 | VHQADICLPRWGCLWGDT |
| 346 | VLLGDICLPLWGCLWGED |
| 347 | VNWGDICLPRWGCLWGES |
| 348 | VVWSDICLPRWGCLWDKE |
| 349 | VWYKDICLPRWGCLWEAE |
| 350 | WDYGDICLPRWGCLWEEG |
| 351 | WEVQDICLPRWGCLWGDD |
| 352 | YIWRDICLPRWGCLWEGE |
| 353 | YRDYDICLPRWGCLWDER |
| 354 | AFWSDICLPRWGCLWEED |
| 355 | DWGRDICLPRWGCLWDEE |
| 356 | EAWGDICLPRWGCLWELE |
| 357 | LILSDICLPRWGCLWDDT |
| 358 | LKLEDICLPRWGCLWGES |
| 359 | LLTRDICLPKWGCLWGSD |
| 360 | LRWSDICLPRWGCLWEET |
| 361 | LYLRDICLPKWGCLWEAD |
| 362 | NWYDDICLPRWGCLWDVE |
| 363 | QDWEDICLPRWGCLWGD |
| 364 | QSWPDICLPKWGCLWGEG |
| 365 | TLLQDICLPRWGCLWESD |
| 366 | VRLMDICLPRWGCLWGEE |
| 367 | VRWEDICLPRWGCLWGEE |
| 368 | WDVADICLPRWGCLWAED |
| 369 | WHMGDICLPRWGCLWSEV |
| 370 | WKDFDICLPRWGCLWDDH |
| 371 | WLSEDICLPQWGCLWEES |
| 372 | WLSEDICLPRWGCLWAAD |
| 373 | WLSDDICLPRWGCLWDDL |
| 374 | EVREWDICLPRWGCLWENWR |
| 375 | FGQEWDICLPRWGCLWGNEQ |
| 376 | IWQLEDICLPRWGCLWEDGL |
| 377 | NTPTYDICLPRWGCLWGDVP |
| 378 | QPVWSDICLPRWGCLWGEDH |
| 379 | SWYGGDICLP-WGCLWSEES |
| 380 | WGMARDWCLPMWGCLWRGGG |
| 381 | WHLTDDICLPRWGCLWGDEQ |
| 382 | NWAENDICLPRWGCLWGDEN |
| 383 | SAREWDICLPTWGCLWEKDI |
| 384 | AGEWDICLPRWGCLWDVE |
| 385 | EIRWDFCLPRWGCLWDED |
| 386 | ESLGDICLPRWGCLWGSG |
| 387 | EYWGDICLPRWGCLWDWQ |
| 388 | KMWSDICLPRWGCLWEEE |
| 389 | MGTKDICLPRWGCLWAEA |
| 390 | MHEWDICLPRWGCLWESS |
| 391 | RGLHDACLPWWGCLWAGS |
| 392 | RLFGDICLPRWGCLWQGE |
| 393 | SGEWDICLPRWGCLWGEG |
| 394 | SMFFDHCLPMWGCLWAEQ |
| 394 | VGEWDICLPNWGCLWERE |
| 396 | WWMADRCLPLWGCLWRGD |
| 397 | WWVRDLCLPTWGCLWSGK |
| 398 | YFDGDICLPRWGCLWGSD |
| 399 | TLFQDICLPRWGCLWEES |
| 400 | WFPKDRCLPVWGCLWERH |
| 401 | QRLMEDICLPRWGCLWEDDF |
| 402 | RLIEDICLPRWGCLWEDD |
| 403 | QRLMEDICLPRWGCLWE |
| 404 | GEWWEDICLPRWGCLWEEED |
| 405 | QRLIEDICLPRWGCLWEDDF |
| 406 | RLIEDICLPRWGCLWED |
| 407 | RLIEDICLPRWGCLWE |
| 408 | RLIEDICLPRWGCLW |
| 409 | RLIEDICLPRWGCL |
| 410 | RLIEDICLPRWGC |
| 411 | LIEDICLPRWGCLWED |
| 412 | IEDICLPRWGCLWED |
| 413 | EDICLPRWGCLWED |
| 414 | DICLPRWGCLWED |
| 415 | ICLPRWGCLWED |
| 416 | CLPRWGCLWED |
| 417 | IEDICLPRWGCLWE |
| 418 | EDICLPRWGCLW |
| 419 | DICLPRWGCL |
| 420 | ICLPRWGCLW |
| 421 | ICLPRWGC |
| 422 | GGGS |
| 423 | DXCLPXWGCLW |
| 424 | X₄DICLPRWGCLWX₃ |
| 425 | X₅DICLPRWGCLWX₄ |
| 426 | XXEMCYFPGICWMXX |
| 427 | XXDLCLRDWGCLWXX |
| 428 | Light Chain variable sequence described |
| | in Figure 4 |
| 429 | Heavy Chain variable sequence described |
| | in Figure 4 |

TABLE 1
Species Specificity of Albumin-Bindine Phage Peptides

| | |
|---|---|
| SEQ | |
| ID | Phage Binding |

| | | | | |
|---|---|---|---|---|
| NO: | Library | Rabbit | Human | Rat |

Selected on Rabbit SA

| | | | | | |
|---|---|---|---|---|---|
| 27 | BA | G E N W C D S T L M A Y D L C G Q V N M | +++ | − | − |
| 28 | BB | M D E L A F Y C G I W E C L M H Q E Q K | +++ | − | − |
| 29 | BC | D L C D V D F C W F | +++ | − | − |
| 30 | BD | K S C S E L H W L L V E E C L F | +++ | − | − |

| | | | | | |
|---|---|---|---|---|---|
| Selected on Human SA | | | | | |
| 31 | HA | E V R S F C T D W P A E K S C K P L R G | − | +++ | − |
| 19 | HB | R A P E S F V C Y W E T I C F E R S E Q | − | ++ | (+) |
| 20 | HC | E M C Y F P G I C W M | − | +++ | ++ |
| 32 | HE | C E V A L D A C R G G E S G C C R H I C E L I R Q L C | − | (+) | − |
| Selected on Rat SA | | | | | |
| 33 | RA | R N E D P C V V L L E M G L E C W E G V | − | − | +++ |
| 34 | RD | D T C V D L V R L G L E C W G | − | − | +++ |
| 35 | RB | Q R Q M V D F C L P Q W G C L W G D G F | ++ | + | +++ |
| 7 | RC | D L C L R D W G C L W | − | − | +++ |
| 36 | RE | C G C V D V S D W D C W S E C L W S H G A | − | − | +++ |

TABLE 2

| | |
|---|---|
| SEQ | Binds |

| | | | |
|---|---|---|---|
| ID | Human | Rabbit | Rat |

| | | | | | |
|---|---|---|---|---|---|
| | Sequences Selected on Rabbit Albumin | | | | |
| Library BA | G E N W C D S T L M A Y D L C G O V N M | 27 | | | |
| BA-B44 | G E D W C D S T L L A F D L C G E G A R | 37 | − | +++ | − |
| BA-B37 | G E N W C D W V L L A Y D L C G E D N T | 38 | − | +++ | − |
| BA-B39 | M E L W C D S T L M A Y D L C G D F N M | 39 | − | +++ | − |
| | Sequences Selected on Human Albumin | | | | |
| Library HA | E V R S F C T D W P A E K S C K P L R G | 31 | | | |
| HA-H74 | E V R S F C T D W P A H Y S C T S L Q G | 40 | +++ | − | − |
| HA-H83 | G - R S F C M D W P A H K S C T P L M L | 41 | +++ | − | − |
| HA-H73 | G V R T F C Q D W P A H N S C K L L R G | 42 | +++ | − | − |
| HA-H76 | Q T R S F C A D W P R H E S C K P L R G | 43 | +++ | − | − |
| HA-H84 | R - R T - C - D W P - H N S C K - L R G | 44 | +++ | − | − |
| Library HB | R A P E S F V C Y W E T I C F E R S E Q | 19 | | | |
| HB-H2 | R A A E S S V C Y W P G I C F D R T E Q | 45 | +++ | − | − |
| HB-H8 | M E P S R S V C Y A E G I C F D R G E Q | 46 | +++ | − | − |
| HB-H3 | R E P A S L V C Y F E D I C F V R A E A | 47 | + | − | − |
| HB-H6 | R G P D - V - C Y W P S I C F E R S M P | 48 | + | − | − |
| HB-H4 | L V P E R I V C Y F E S I C Y E R S E L | 49 | + | − | − |
| HB-H16 | R M P A S L P C Y W E T I C Y E S S E Q | 50 | + | − | − |
| HB-H18 | R T A E S L V C Y W P G I C F A Q S E R | 51 | + | − | − |
| HB-H1 | R A P E R W V C Y W E G I C F D R Y E Q | 52 | (+) | − | − |
| Library HC | E M C Y F P G I C W M | 20 | | | |
| HB-H12 | E I C Y F P G I C W I | 53 | ++ | − | − |
| HB-H13 | E L C Y F P G I C W T | 54 | ++ | − | − |
| HC-H6 | D I C Y I P G I C W M | 55 | ++ | − | − |
| HC-H2 | K L C Y F P G I C W S | 56 | ++ | − | − |
| HC-H3 | D L C Y F P G I C W M | 57 | ++ | − | − |
| HC-H4 | G M C Y F P G I C W A | 58 | ++ | − | − |
| HC-H7 | E M C Y F P G I C W S | 59 | ++ | − | − |
| HC-H9 | E M C Y F P G I C W T | 60 | ++ | − | − |
| HC-H10 | K T C Y F P G I C W M | 61 | ++ | − | − |
| HC-H5 | K V C Y F P G I C W M | 62 | | | |
| HC-H8 | D V C Y F P G I C W M | 63 | ++ | − | − |
| HC-H17 | E I C Y F P G I C W M | 64 | ++ | − | − |
| HC-H14 | A L C Y F P G I C W M | 65 | ++ | − | − |
| HC-H15 | E L C Y F P G I C W P | 66 | ++ | − | − |
| HC-H20 | E L C Y F P G I C W M | 67 | ++ | − | − |
| HC-H13 | D M C Y F P G I C W L | 68 | ++ | − | − |
| HC-H18 | D M C Y F P G I C F N | 69 | ++ | − | − |
| HC-H12 | E T C Y F P G I C W L | 70 | ++ | − | − |
| HC-H11 | E V C Y F P G I C W F | 71 | ++ | − | − |
| HC-H16 | E V C Y F P G I C W E | 72 | ++ | − | − |
| HC-H19 | E V C Y F P G I C W M | 73 | ++ | − | − |
| Library HBC | X X E M C Y F P G I C W M X X | 426 | | | |
| HBC-H7 | L A E M C Y F P G I C W M S A | 74 | +++ | − | − |
| HBC-H4 | G G E I C Y F P G I C R V L P | 75 | +++ | − | − |
| HBC-H6 | E H D M C Y F P G I C W I A D | 76 | +++ | − | − |
| HBC-H10 | V Q E V C Y F P G I C W M Q E | 77 | +++ | − | − |
| HBC-H2 | S R E V C Y Y P G I C W N G A | 78 | +++ | − | − |
| HBC-H1 | D S E V C Y F P G I C W S G T | 79 | +++ | − | − |
| HBC-H3 | G T E V C Y F P G I C W G G G | 80 | +++ | − | − |
| HBC-H8 | S Y A P C Y F P G I C W M G N | 81 | +++ | − | − |
| HBC-H17 | H A E I C Y F P G I C W T E R | 82 | +++ | − | − |
| HBC-H11 | N D E I C Y F P G V C W K S G | 83 | +++ | − | − |
| HBC-H18 | R D T V C Y F P G I C W M A S | 84 | +++ | − | − |
| HBC-H19 | V R D M C Y F P G I C W K S E | 85 | +++ | − | − |
| HBC-H12 | A S E I C Y F P G I C W M V E | 86 | +++ | − | − |
| HBC-H13 | Q T E L C Y F P G I C W N E S | 87 | +++ | − | − |
| HBC-H14 | T T E M C Y F P G I C W K T E | 88 | +++ | − | − |
| HBC-H15 | K T E I C Y F P G I C W M S G | 89 | +++ | − | − |
| HBC-H16 | Q - - - C - F P G - - W V - K | 90 | +++ | − | − |
| HB-H10 | I V E M C Y Y P G I C W I S P | 91 | +++ | − | − |
| HB-H7 | S G A I C Y V P G I C W T H A | 92 | +++ | − | − |
| | Sequences Selected on Rat Albumin | | | | |
| Library RB | Q R Q M V D F C L P Q W G C L W G D G F | 35 | | | |
| RB-H1 | Q R H P E D I C L P R W G C L W G D D D | 93 | ++ | +++ | +++ |
| RB-H6 | N R Q M E D I C L P Q W G C L W G D D F | 94 | ++ | +++ | +++ |
| RB-B2 | Q R L M E D I C L P R W G C L W G D R F | 95 | ++ | +++ | +++ |
| RB-B5 | Q W H M E D I C L P Q W G C L W G D V L | 96 | ++ | +++ | +++ |
| RB-B6 | Q W Q M E N V C L P K W G C L W E E L D | 97 | ++ | +++ | +++ |
| RB-B4 | L W A M E D I C L P K W G C L W E D D F | 98 | ++ | +++ | +++ |
| RB-B7 | L R L M D N I C L P R W G C L W D D G F | 99 | ++ | +++ | |
| RB-B8 | H S Q M E D I C L P R W G C L W G D E L | 100 | ++ | +++ | +++ |
| RB-B11 | Q W Q V M D I C L P R W G C L W A D E Y | 101 | ++ | +++ | +++ |
| RB-B12 | Q G L I G D I C L P R W G C L W G D S V | 11 | ++ | +++ | +++ |
| RB-B16 | H R L V E D I C L P R W G C L W G N D F | 102 | ++ | +++ | +++ |
| RB-B9 | Q M H M M D I C L P K W G C L W G D T S | 103 | (+) | +++ | +++ |
| RB-B14 | L R I F E D I C L P K W G C L W G E G F | 104 | (+) | +++ | +++ |
| RB-B3 | Q S Y M E D I C L P R W G C L S D D A S | 105 | (+) | +++ | +++ |
| RB-B10 | Q G D F W D I C L P R W G C L S G E G Y | 106 | − | +++ | +++ |
| RB-B1 | R W Q T E D V C L P K W G C L F G D G V | 107 | − | +++ | ++ |
| RB-R8 | Q G L I G D I C L P R W G C L W G D S V | 11 | ++ | +++ | +++ |
| RB-R16 | L I F M E D V C L P Q W G C L W E D G V | 108 | ++ | +++ | +++ |
| HC-R10 | Q R D M G D I C L P R W G C L W E D G V | 109 | ++ | +++ | +++ |
| RB-R4 | Q R H M M D F C L P K W G C L W G D G Y | 110 | − | (+) | +++ |
| RB-R7 | Q R P I M D F C L P K W G C L W E D G F | 111 | − | (+) | +++ |
| RB-R11 | E R Q M V D F C L P K W G C L W G D G F | 112 | − | (+) | +++ |
| RB-R12 | Q G Y M V D F C L P R W G C L W G D A N | 113 | − | (+) | +++ |
| RB-R13 | K M G R V D F C L P K W G C L W G D E L | 114 | − | (+) | +++ |
| RB-R15 | Q S Q L E D F C L P K W G C L W G D G F | 115 | − | (+) | +++ |
| RB-R17 | Q G G M G D F C L P Q W G C L W G E D L | 116 | − | (+) | +++ |
| RB-R5 | Q R L M W E I C L P L W G C L W G D G L | 117 | − | − | |
| RB-R10 | Q R Q I M D F C L P H W G C L W G D G F | 118 | − | − | |
| RB-R2 | G R Q V V D F C L P K W G C L W E E G L | 119 | − | − | |
| RB-R3 | Q M Q M S D F C L P Q W G C L W G D G Y | 120 | − | − | |
| RB-R9 | K S R M G D F C L P E W G C L W G D E L | 121 | − | − | |
| RB-R1 | E R Q M E D F C L P Q W G C L W G D G V | 122 | − | − | |
| RB-R14 | Q R Q V V D F C L P Q W G C L W G D G S | 123 | − | − | |
| Library RC | D L C L R D W G C L W | 7 | | | |
| RC-R6 | D I C L P E W G C L W | 124 | − | − | ++ |
| RC-R8 | D I C L P E W G C L W | 124 | − | − | ++ |
| RO-R15 | D I C L P E W G C L W | 124 | − | − | ++ |
| RC-R1 | D I C L P V W G C L W | 125 | − | − | ++ |
| RC-R2 | D I C L P V W G C L W | 125 | − | − | ++ |
| RC-R3 | D I C L P V W G C L W | 125 | − | − | ++ |
| RC-R10 | D I C L P V W G C L W | 125 | − | − | ++ |
| RC-R12 | D I C L P V W G C L W | 125 | − | − | ++ |
| RO-R18 | D I C L P V W G C L W | 125 | − | − | ++ |
| RC-R9 | D L C L P E W G C L W | 126 | − | − | (+) |
| RC-R4 | D L C L P K W G C L W | 127 | − | − | ++ |
| RC-R5 | D L C L P V W G C L W | 128 | − | − | (+) |
| RC-R20 | D I C L P A W G C L W | 129 | − | − | ++ |
| RC-R17 | D I C L P D W G C L W | 130 | − | − | ++ |
| RC-R13 | D I C L P R W G C L W | 8 | − | − | ++ |
| RC-R16 | D I C L E R W G C L W | 131 | − | − | ++ |
| Library RBC | X X D L C L R D W G C L W X X | 427 | | | |
| RBC-R16 | E W D V C L P H W G C L W D G | 132 | − | (+) | +++ |
| RBC-R7 | W D D I C F R D W G C L W G S | 133 | − | − | +++ |
| RBC-R1 | M D D I C L H H W G C L W D E | 134 | − | − | +++ |
| RBC-R2 | M D D L C L P N W G C L W G D | 135 | − | − | +++ |
| RBC-R4 | F E D F C L P N W G C L W G S | 136 | − | − | +++ |
| RBC-R6 | F E D L C V V R W G C L W G D | 137 | − | − | +++ |
| RBC-R5 | W E D L C L P D W G C L W E D | 138 | − | − | +++ |
| RBC-R9 | S E D F C L P V W G C L W E D | 139 | − | − | +++ |
| RBC-R10 | D F D L C L P D W G C L W D D | 140 | − | − | +++ |
| RBC-R8 | N W D L C F P D W G C L W D D | 141 | − | − | +++ |
| RBC-R14 | E E D L C L P V W G C L W G A | 142 | − | − | +++ |
| RBC-R20 | E E D V C L P V W G C L W E G | 143 | − | − | +++ |
| RBC-R12 | M F D L C L P K W G C L W G N | 144 | − | − | +++ |
| RBC-R13 | E F D L C L P T W G C L W E D | 145 | − | − | +++ |
| RBC-R15 | M W D V C F P D W G C L W D V | 146 | − | − | +++ |
| RBC-R18 | E W D V C F P A W G C L W D Q | 147 | − | − | +++ |
| RBC-R11 | V W D L C L P Q W G C L W D E | 148 | − | − | +++ |
| Library RD | D T C V D L V R L G L E C W G | 34 | | | |
| RD-R2 | D T C A D L V R L G L E C W A | 149 | − | − | +++ |
| RD-R7 | N T C A D L V R L G L E C W A | 150 | − | − | +++ |
| RD-R11 | D T C 0 0 L V 0 L G L E C W A | 151 | − | − | +++ |
| RD-R5 | D T C E D L V R L G L E C W A | 152 | − | − | +++ |
| RD-R6 | D S C G D L L R L G L E C W A | 153 | − | − | +++ |
| RD-R1 | D T C S D L V G L G L E C W A | 154 | − | − | +++ |

Table 3
Multi Species Binders

Binds

| | | | | |
|---|---|---|---|---|
| | SEQ ID | | | |
| Phage | NO: | Human | Rabbit | Rat |

| | | | | | |
|---|---|---|---|---|---|
| RB | Q R Q M V D F C L P Q W G C L W G D G F | 35 | + | ++ | +++ |
| RB-H1 | Q R H P E D I C L P R W G C L W G D D D | 93 | ++ | +++ | +++ |
| RB-H6 | N R Q M E D I C L P Q W G C L W G D D F | 94 | ++ | +++ | +++ |
| RB-B12 | Q G L I G D I C L P R W G C L W G D S V | 11 | ++ | +++ | +++ |
| RB-B8 | H S Q M E D I C L P R W G C L W G D E L | 100 | ++ | +++ | +++ |
| RB-B7 | L R L M D N I C L P R W G C L W D D G F | 99 | ++ | +++ | +++ |
| RB-B5 | Q W H M E D I C L P Q W G C L W G D V L | 96 | ++ | +++ | +++ |
| RB-B6 | Q W Q M E N V C L P K W G C L W E E L D | 97 | ++ | +++ | +++ |
| RB-B4 | L W A M E D I C L P K W G C L W E D D F | 98 | ++ | +++ | +++ |
| RB-B11 | Q W Q V M D I C L P R W G C L W A D E Y | 101 | ++ | +++ | +++ |
| RB-B16 | H R L V E D I C L P R W G C L W G N D F | 102 | ++ | +++ | +++ |
| RB-B2 | Q R L M E D I C L P R W G C L W G D R F | 95 | ++ | +++ | +++ |
| RB-R8 | Q G L I G D I C L P R W G C L W G D S V | 11 | ++ | +++ | +++ |
| RB-R16 | L I F M E D V C L P Q W G C L W E D G V | 108 | ++ | +++ | +++ |
| HC-R10 | Q R D M G D I C L P R W G C L W E D G V | 109 | ++ | +++ | +++ |

TABLE 4
Sequences Selected on Rat Albumin

| SEQ ID | |
|---|---|
| NO: | Hard Randomization Library |
| 155 | X X X X X D X C L P X W G C L W X X X X |
| 157 | A A Q V G D I C L P R W G C L W S E Y A |
| 8 | A G W A A D V C L P R W G C L W E E D V |
| 9 | A S V V D D I C L P V W G C L W G E D I |
| 160 | A T M E D D I C L P R W G C L W G A E E |
| 161 | D E D F E D Y C L P P W G C L W G S S M |
| 162 | E G T W D D F C L P R W G C L W L G E R |
| 163 | E R W E G D V C L P R W G C L W G E S G |
| 164 | G D W M H D I C L P K W G C L W D E K A |
| 165 | G I E W G D T C L P K W G C L W R V E G |
| 166 | G Q Q G E D V C L P V W G C L W D T S S |
| 167 | G R Y P M D L C L P R W G C L W E D S A |
| 168 | G S A G D D L C L P R W G C L W E R G A |
| 169 | H A S D W D V C L P G W G C L W E E D D |
| 170 | L G V T H D T C L P R W G C L W D E V G |
| 171 | L V W E E D F C L P K W G C L W G A E D |
| 172 | N V G W N D I C L P R W G C L W A Q E S |
| 173 | Q G V E W D V C L P Q W G C L W T A E V |
| 174 | R L D A W D I C L P Q W G C L W E E P S |
| 175 | S E A P G D Y C L P R W G C L W A Q E K |
| 176 | T A M D E D V C L P R W G C L W G S G S |
| 177 | T E I G Q D F C L P R W G C L W V P G T |
| 178 | T L G W P D F C L P K W G C L W R E S D |
| 179 | T L S N Q D I C L P G W G C L W G G I N |
| 180 | T S T G G D L C L P R W G C L W D S S E |
| 181 | V S E M D D I C L P L W G C L W A D A P |
| 182 | V S E W E D I C L P S W G C L W E T Q D |
| 183 | V V G D G D F C L P K W G C L W D Q A R |
| 184 | V V W D D D V C L P R W G C L W E E Y G |
| 185 | W S D S D D V C L P R W G C L W G N V A |
| 186 | W V E E G D I C L P R W G C L W E S V E |
| 187 | A Q A M G D I C L P R W G C L W E A E I |
| 188 | A S D R G D L C L P Y W G C L W G P D G |
| 189 | A S D P G D V C L P R W G C L W G E S F |
| 190 | A S N W E D V C L P R W G C L W G E R N |
| 191 | A S T P R D I C L P R W G C L W S E D A |
| 192 | D G E E G D L C L P R W G C L W A L E H |
| 193 | E G E E V D I C L P Q W G C L W G Y P V |
| 194 | E V G D L D L C L P R W G C L W G N D K |
| 195 | F R D G E D F C L P Q W G C L W A D T S |
| 196 | G D M V N D F C L P R W G C L W G S E N |
| 197 | G R M G T D L C L P R W G C L W G E V E |
| 198 | H E W E R D I C L P R W G C L W R D G D |
| 199 | K K V S G D I C L P I W G C L W D N D Y |
| 200 | L L E S D D I C L P R W G C L W H E D G |
| 201 | M Q A E S D F C L P H W G C L W D E G T |
| 202 | M Q G P L D I C L P R W G C L W G G V D |
| 203 | Q M P L E D I C L P R W G C L W E G R E |
| 204 | R E E W G D L C L P T W G C L W E T K K |
| 205 | R V W T E D V C L P R W G C L W S E G N |
| 206 | S I R E Y D V C L P K W G C L W E P S A |
| 207 | S P T E W D M C L P K W G C L W G D A L |
| 208 | S S G L E D I C L P N W G C L W A D G S |
| 209 | S V G W G D I C L P V W G C L W G E G G |
| 210 | T E E N W D L C L P R W G C L W G D D W |
| 211 | T S G S D D I C L P V W G C L W G E D S |
| 212 | T W P — G D L C L P R W G C L W E A E S |
| 213 | W D H E L D F C L P V W G C L W A E D V |
| 214 | W T E S E D I C L P G W G C L W G P E V |
| 215 | W V P F E D V C L P R W G C L W S S Y Q |
| 156 | X X X X D X C L P X W G C L W X X X |
| 216 | E E D S D I C L P R W G C L W N T S |
| 217 | E G Y W D L C L P R W G C L W E L E |
| 218 | E L G E D L C L P R W G C L W G S E |
| 219 | E T W S D V C L P R W G C L W G A S |
| 220 | G D Y V D L C L P G W G C L W E D G |
| 221 | G V L D D I C L P R W G C L W G P K |
| 222 | H M M D D V C L P G W G C L W A S E |
| 223 | I D Y T D L C L P A W G C L W E L E |
| 224 | I E H E D L C L P R W G C L W A V D |
| 225 | I S E W D L C L P R W G C L W D R S |
| 226 | I S W A D V C L P K W G C L W G K D |
| 227 | I S W G D L C L P R W G C L W E G S |
| 228 | K L W D D I C L P R W G C L W S P L |
| 229 | L A W P D V C L P R W G C L W G G M |
| 230 | L N E S D I C L P T W G C L W G V D |
| 231 | L P E Q D V C L P V W G C L W D A N |
| 232 | M A W G D V C L P R W G C L W A G G |
| 233 | N E E W D V C L P R W G C L W G G V |
| 234 | Q E L Q D F C L P R W G C L W G V G |
| 235 | Q R E W D V C L P R W G C L W S D V |
| 236 | Q R F W D T C L P R W G C L W G G D |
| 237 | R V F T D V C L P R W G C L W D L G |
| 238 | S G W D D V C L P V W G C L W G P S |
| 239 | S S A S D Y C L P R W G C L W G D L |
| 240 | S W Q G D I C L P R W G C L W G V D |
| 241 | S Y E T D V C L P Y W G C L W E D A |
| 242 | S Y W G D V C L P R W G C L W S E A |
| 243 | T L E W D M C L P R W G C L W T E Q |
| 244 | V G E F D I C L P R W G C L W D A E |
| 245 | V T S W D V C L P R W G C L W E E D |
| 246 | W L W E D L C L P K W G C L W E E D |
| 247 | A L F E D V C L P V W G C L W G G E |
| 248 | A S E W D V C L P T W G C L W M E G |
| 249 | A Y S A D I C L P R W G C L W M S E |
| 156 | X X X X D X C L P X W G C L W X X X |
| 250 | E D W E D I C L P Q W G C L W E G M |
| 251 | E D W T D L C L P A W G C L W D T E |
| 252 | E E W E D L C L P R W G C L W S A E |
| 253 | E F W Q D I C L P N W G C L W A E S |
| 254 | E G F S D I C L P R W G C L W S Q E |
| 255 | E T W E D L C L P N W G C L W D L E |
| 256 | G E V N D F C L P R W G C L W E G D |
| 257 | G G E W D V C L P A W G C L W G E E |
| 258 | K D W Y D I C L P R W G C L W G G E |
| 259 | K L G Q D I C L P R W G C L W D F A |
| 260 | L E E W D I C L P Q W G C L W R E G |
| 261 | L V L P D I C L P K W G C L W G D T |
| 262 | M D L A D I G L P K W G C L W E S D |
| 263 | M V L D D I G L P R W G C L W S E K |
| 264 | M W S G D L C L P R W G C L W G E T |
| 265 | N R M G D I C L P R W G C L W D G H |
| 266 | R D W E D L C L P N W G C L W E L S |
| 267 | R G D W D L C L P K W G C L W E G V |
| 268 | R Q W E D I C L P R W G C L W G V G |
| 269 | R V E Y D L C L P R W G C L W E P P |
| 270 | S I W S D I C L P R W G C L W E S D |
| 271 | T D E W D I C L P N W G C L W E A G |
| 272 | T E D V D F C L P L W G C L W E E P |
| 273 | V K E E D F C L P R W G C L W E A G |
| 274 | W D F E D I C L P R W G C L W A D M |
| 275 | W E D W D V C L P R W G C L W G G G |
| 276 | Y E D I D I C L P R W G C L W D L S |

TABLE 5
Sequences Selected on Rabbit Albumin

| SEQ ID | |
|---|---|
| NO: | Hard Randomization Library |
| 155 | X X X X X D X C L P X W G C L W X X X X |
| 277 | A G L D E D I C L P R W G C L W G K E A |
| 278 | A G M M G D I C L P R W G C L W Q G E P |
| 279 | A P G D W D F C L P K W G C L W D D D A |
| 280 | A D L F D D I C L P R W G C L W S D G Y |
| 281 | A R T M G D I C L P R W G C L W G A S D |
| 282 | A W D D F D V C L P R W G C L W E P E S |
| 283 | D T T W G D I C L P R W G C L W S E E A |
| 284 | E G F L G D I C L P R W G C L W G H Q A |
| 285 | E D W L H D I C L P K W G C L W D D T D |
| 286 | E T G W P D I C L P R W G C L W E E G E |
| 287 | F E L G E D I C L P R W G C L W E E H N |
| 288 | G A S L G D I C L P R W G C L W G P E D |
| 289 | G E W W E D I C L P R W G C L W G S S S |
| 290 | G S L E S D I C L P R W G C L W G I D E |
| 291 | G W L E E D I C L P K W G C L W G A D N |
| 292 | H E D W D D I C L P R W G C L W G G S Y |
| 293 | D R V D D D I C L P R W G C L W G E N S |
| 294 | 5 V G W G D I C L P K W G C L W A E S D |
| 295 | T L M S N D I C L P R W G C L W D E P K |
| 296 | T L V L D D I C L P R W G C L W D M T D |
| 297 | T W D G E D I C L P R W G C L W D T E V |
| 298 | V G V F D D I C L P R W G C L W E D P V |
| 299 | V P A M G D I C L P R W G C L W E A R N |
| 300 | V S L G D D I C L P K W G C L W E P E A |
| 301 | V W I D R D I C L P R W G C L W D T E N |
| 302 | W R W N E D I C L P R W G C L W E E E A |
| 303 | A V S W A D I C L P R W G C L W E R A D |
| 304 | A W L D E D I C L P K W G C L W N T G V |
| 305 | F S L D E D I C L P K W G C L W G A E K |
| 306 | G D L G D D I C L P R W G C L W D E Y P |
| 307 | G E G W S D I C L P R W G C L W A E D E |
| 308 | G L M G E D I C L P R W G C L W K G D I |
| 155 | X X X X D X C L P X W G C L W X X X X |
| 309 | G W H D R D I C L P R W G C L W E D N D |
| 310 | L L G G H D I C L P R W G C L W G G D V |
| 311 | M R W S S D I C L P K W G C L W G D E E |
| 312 | D F E W D D I C L P R W G C L W E V E V |
| 313 | D G W W H D I C L P R W G C L W E E G E |
| 314 | R E G W P D I C L P R W G C L W S E T G |
| 315 | R E L W G D I C L P R W G C L W E H A T |
| 316 | R L E L M D I C L P R W G C L W D P D D |
| 317 | S G V L G D I C L P R W G C L W E E A G |
| 318 | S L G L T D L C L P R W G C L W E E E D |
| 319 | S S L E Q D I C L P R W G C L W G D D A |
| 320 | S V L S D D I C L P R W G C L W W D F S |
| 321 | T S L L D D I C L P R W G C L W Y E E G |
| 322 | T S L A D D I C L P R W G C L W S E D G |
| 323 | V E M W H D I C L P R W G C L W D S N A |
| 324 | W D L A S D I C L P R W G C L W E E E A |
| 325 | F I T Q D I C L P R W G C L W G E N |
| 326 | F L W R D I C L P R W G C L W S E G |
| 327 | F V H E D I C L P R W G C L W G E G |
| 328 | G L G D D I C L P R W G C L W G R D |
| 329 | G M F D D I C L P K W G C L W G L G |
| 330 | G P G W D I C L P R W G C L W G E E |
| 331 | G P W Y D I C L P R W G C L W D G V |
| 332 | G W D D D I C L P R W G C L W G D G |
| 333 | L E Y E D I C L P K W G C L W G G E |
| 334 | L L D E D I C L P R W G C L W G V R |
| 335 | L M S P D I C L P K W G C L W E G D |
| 336 | L V L G D I C L P R W G C L W E S D |
| 337 | M L S R D I C L P R W G C L W E E E |
| 338 | M P W T D I C L P R W G C L W S E S |
| 339 | R L G S D I C L P R W G C L W G A G |
| 340 | R L G S D I C L P R W G C L W D Y Q |
| 341 | S P W M D I C L P R W G C L W E S G |
| 342 | S T F T D I C L P R W G C L W E L E |
| 343 | S V L S D I C L P R W G C L W E E S |
| 344 | T W F S D I C L P R W G C L W E P G |
| 345 | V H Q A D I C L P R W G C L W G D T |
| 155 | X X X X X D X C L P X W G C L W X X X X |
| 346 | V L L G D I C L P L W G C L W G E D |
| 347 | V N W G D I C L P R W G C L W G E S |
| 348 | V V W S D I C L P R W G C L W D K E |
| 349 | V W Y K D I C L P R W G C L W E A E |
| 350 | W D Y G D I C L P R W G C L W E E G |
| 351 | W E V Q D I C L P R W G C L W G D D |
| 352 | Y I W R D I C L P R W G C L W E G E |
| 353 | Y R D Y D I C L P R W G C L W D E R |
| 354 | A F W S D I C L P R W G C L W E E D |
| 355 | D W G R D I C L P R W G C L W D E E |
| 356 | E A W G D I C L P R W G C L W E L E |
| 357 | L I L S D I C L P R W G C L W D D T |
| 358 | L K L E D I C L P R W G C L W G E S |
| 359 | L L T R D I C L P K W G C L W G S D |
| 360 | L R W S D I C L P R W G C L W E E T |
| 361 | L Y L R D I C L P K W G C L W E A D |
| 362 | N W Y D D I C L P R W G C L W D V E |
| 363 | Q D W E D I C L P R W G C L W G D — |
| 364 | Q S W P D I C L P K W G C L W G E G |
| 365 | T L L Q D I C L P R W G C L W E S D |
| 366 | V R L M D I C L P R W G C L W G E E |
| 367 | V R W E D I C L P R W G C L W G E E |
| 368 | W D V A D I C L P R W G C L W A E D |
| 369 | W H M G D I C L P R W G C L W S E V |
| 370 | W K D F D I C L P R W G C L W D D H |
| 371 | W L S E D I C L P Q W G C L W E E S |
| 372 | W L S E D I C L P R W G C L W A A D |
| 373 | W L S D D I C L P R W G C L W D D L |

TABLE 6
Sequences Selected on Human Albumin

| | |
|---|---|
| SEQ ID | |
| NO: | Hard Randomization Library |

| | |
|---|---|
| 155 | X X X X X D X C L P X W G C L W X X X X |
| 374 | E V R E W D I C L P R W G C L W E N W R |
| 375 | F G D E W D I C L P R W G C L W G N E Q |
| 376 | I W Q L E D I C L P R W G C L W E D G L |
| 377 | N T P T Y D I C L P R W G C L W G D V P |
| 378 | Q P V W S D I C L P R W G C L W G E D H |
| 379 | S W Y G G D I C L P - W G C L W S E E S |
| 380 | W G M A R D W C L P M W G C L W R G G G |
| 381 | W H L T D D I C L P R W G C L W G D E Q |
| 382 | N W A E N D I C L P R W G C L W G D E N |
| 383 | S A R E W D I C L P T W G C L W E K D I |
| 156 | X X X X D X C L P X W G C L W X X X |
| 384 | A G E W D I C L P R W G C L W D V E |
| 385 | E I R W D F C L P R W G C L W D E D |
| 386 | E S L G D I C L P R W G C L W G S G |
| 387 | E Y W G D I C L P R W G C L W D W Q |
| 388 | K M W S D I C L P R W G C L W E E E |
| 389 | M G T K D I C L P R W G C L W A E A |
| 390 | M H E W D I C L P R W G C L W E S S |
| 391 | R G L H D A C L P W W G C L W A G S |
| 392 | R L F G D I C L P R W G C L W Q G E |
| 393 | S G E W D I C L P R W G C L W G E G |
| 394 | S M F F D H C L P M W G C L W A E Q |
| 395 | V G E W D I C L P N W G C L W E R E |
| 396 | W W M A D R C L P L W G C L W R G D |
| 397 | W W V R D L C L P T W G C L W S G K |
| 398 | Y F D G D I C L P R W G C L W G S D |
| 399 | T L F Q D I C L P R W G C L W E E S |
| 400 | W F P K D R C L P V W G C L W E R H |

TABLE 7
Peptides Binding Multiple Species Albumin

| Pep- | SEQ ID | IC₅₀ (nM) | | |
|---|---|---|---|---|
| tide | NO: | Rabbit | Rat | Mouse |

| | | | | | |
|---|---|---|---|---|---|
| SA02 | 7 | D L C L R D W G C L W -n | | | |
| SA04 | 8 | D I C L P R W G C L W -n | 8543 | 787 | 40 |
| SA05 | 16 | M E D I C L P R W G C L W E D -n | 804 | 161 | 6 |
| SA06 | 401 | Q R L M E D I C L P R W G C L W E D D F -n | 128 | 68 | 8 |
| SA07 | 11 | Q G L I G D I C L P R W G C L W G D S V -n | 30 | 35 | 6 |
| SA08 | 12 | Ac Q G L I G D I C L P R W G C L W G D S V K -n | 63 | 68 | 10 |
| SA09 | 13 | Ac E D I C L P R W G C L W E D D -n | 1687 | 258 | 6 |
| SA10 | 14 | Ac R L M E D I C L P R W G C L W E D D -n | 86 | 77 | 4 |
| SA11 | 15 | Ac M E D I C L P R W G C L W E D D -n | 1213 | 232 | 17 |
| SA12 | 16 | Ac M E D I C L P R W G C L W E D -n | 1765 | 205 | 13 |
| SA13 | 17 | Ac R L M E D I C L A R W G C L W E D D -n | 3200 | 2480 | 188 |
| D3H44-L | 401 | Q R L M E D I C L P R W G C L W E D D F -n | 241 | | |
| D3H44-Ls | 401 | Q R L M E D I C L P R W G C L W E D D F -n | 75 | | |

TABLE 8

| | |
|---|---|
| Surface | Peptide |
| Plasmon Resonance | Competition |
| Kd (nN) | IC₅₀ (nM) |

| | | | | | | | |
|---|---|---|---|---|---|---|---|
| HuSA | BuSA | RSA | SA | ID | SEQUENCE | BuSA | MuSA |

| | | | | | | | |
|---|---|---|---|---|---|---|---|
| 467 ± 47 | 320 ± 22 | 266 ± 6 | 21 | 402 | _{Ac-}RLIEDICLPRWGCLWEDD_{-NH2} | 270 ± 110 | 7 ± 2 |
| 803 ± 82 | 143 ± 5 | 229 ± 9 | 06 | 403 | QRLMEDICLPRWGCLWE | 130 ± 50 | 6 ± 2 |
| 858 ± 59 | 108 ± | 158 ± 3 | 08 | 11 | _{AC-}QGLIGDICLPRWGCLWGDSVK_{-NH2} | 51 ± 11 | 12 ± 2 |
| 878 ± 58 | 65 ± 3 | 150 ± 5 | 15 | 404 | GEWWEDICLPRWGCLWEEED-NH2 | 13 ± 2 | 5 ± 1 |

TABLE 9

| | | | |
|---|---|---|---|
| | SEQ | | |
| PEP- | ID | RSA | |
| TIDE | NO: | SEQUENCE | IC₅₀ (nM) |

| | | | |
|---|---|---|---|
| SA20 | 405 | _{Ac} QRLIEDICLPRWGCLWEDDF _{NH2} | 260 |
| SA21 | 402 | _{Ac} RLIEDICLPRWGCLWEDD_{NH2} | 270 ± 110 |
| SA22 | 406 | _{Ac} RLIEDICLPRWGCLWED_{NH2} | 430 ± 70 |
| SA29 | 407 | _{Ac} RLIEDICLPRWGCLWE_{NH2} | 400 ± 90 |
| SA31 | 408 | _{Ac} RLIEDICLPRWGCLW_{NH2} | 200 |
| SA33 | 409 | _{Ac} RLIEDICLPRWGCL_{NH2} | 4310 ± 2770 |
| SA35 | 410 | _{Ac} RLIEDICLPRWGC_{NH2} | >250000 |
| SA23 | 411 | _{Ac} LIEDICLPRWGCLWED_{NH2} | 360 ± 140 |
| SA24 | 412 | _{Ac} IEDICLPRWGCLWED_{NH2} | 1380 ± 410 |
| SA25 | 413 | _{Ac} EDICLPRWGCLWED_{NH2} | 2730 ± 1300 |
| SA26 | 414 | _{Ac} DICLPRWGCLWED_{NH2} | 3120 ± 660 |
| SA27 | 415 | _{Ac} ICLPRWGCLWED_{NH2} | 86700 ± 21800 |
| SA28 | 416 | _{Ac} CLPRWGCLWED_{NH2} | >400000 |
| SA30 | 417 | _{Ac} IEDICLPRWGCLWE_{NH2} | 1800 ± 590 |
| SA32 | 418 | _{Ac} EDICLPRWGCLW_{NH2} | 2170 ± 520 |
| SA04 | 8 | DICLPRWGCLW_{NH2} | 8540 ± 4620 |
| SA34 | 419 | _{Ac} DICLPRWGCL_{NH2} | 28210 ± 6500 |
| SA19 | 419 | DICLPRWGCL_{NH2} | 24510 ± 2100 |
| SA18 | 420 | ICLPRWGCLW_{NH2} | 124900 |
| SA36 | 421 | _{Ac} ICLPRWGC_{NH2} | >250000 |

TABLE 10
Kd (Solution Binding with Preincubation) ELISA Assay

| | | | |
|---|---|---|---|
| | EC50 Direct Binding | Kd solution phase | |
| Molecules | ELISA | binding | Kd by BIAcore |

Rabbit SA

| | | | | | | |
|---|---|---|---|---|---|---|
| 4D5Fab-H | 25 | nM | 36 | nM | 150 | nM |
| 4D5Fab-H4 | ˜500 | nM | 444 | nM | 500 | nM |
| 4D5Fab-H8 | ˜500 | nM | 247 | nM | 710 | nM |
| 4D5Fab-H10 | >2 | uM | 1065 | nM | | |
| 4D5Fab-H11 | >2 | uM | 1110 | nM | | |

Rat SA

| | | | | | | |
|---|---|---|---|---|---|---|
| 4D5Fab-H | 65 | pM | 92 | nM | 20 | nM |
| 4D5Fab-H4 | 75 | pM | 149 | nM | 40 | nM |
| 4D5Fab-H8 | 45 | pM | 145 | nM | 40 | nM |
| 4D5Fab-H10 | 8,000 | pM | 493 | nM | | |
| 4D5Fab-H11 | >1 | μM | >2 | μM | | |

Mouse SA

| | | | | | | |
|---|---|---|---|---|---|---|
| 4D5Fab-H | 70 | pM | 44 | nM | 20 | nM |
| 4D5Fab-H4 | 77 | pM | 52 | nM | 30 | nM |
| 4D5Fab-H8 | 43 | pM | 41 | nM | 30 | nM |
| 4D5Fab-H10 | 14,520 | pM | 2,500 | nM | | |
| 4D5Fab-H11 | >1 | μM | 1,250 | nM | | |

We claim:

1. A conjugate molecule comprising at least one serum albumin-binding domain (SABM), at least one targeting agent (TA) and at least one cytotoxic agent (CA).

2. The conjugate molecule according to claim 1, wherein the SABM comprises an amino acid sequence that is at least 50% identical to the sequence of DICLPRWGCLW (SEQ ID NO:8) and wherein the amino acid sequence has two Cys residues with five amino acid residues in between the Cys residues.

3. The conjugate molecule according to claim 1, wherein the SABM comprises a variant of the amino acid sequence of DICLPRWGCLW (SEQ ID NO:8), wherein between 1-5 residues of any of one of the residues of SEQ ID NO:8, except for the Cys residues is substituted with a different amino acid residue.

4. The conjugate molecule according to claim 1, wherein the SABM comprises a linear or cyclic amino acid sequence selected from the group consisting of:

| | |
|---|---|
| Xaa-Xaa-Cys-Xaa-Xaa-Xaa-Xaa-Xaa- | |
| Cys-Xaa-Xaa | |
| Phe-Cys-Xaa-Asp-Trp-Pro-Xaa-Xaa- | [SEQ ID NO: 1] |
| Xaa-Ser-Cys | |
| Val-Cys-Tyr-Xaa-Xaa-Xaa-Ile-Cys- | [SEQ ID NO: 2] |
| Phe | |
| Cys-Tyr-Xaal-Pro-Gly-Xaa-Cys | [SEQ ID NO: 3] |
| Asp-Xaa-Cys-Leu-Pro-Xaa-Trp-Gly- | [SEQ ID NO: 4] |
| Cys-Leu-Trp | |
| Trp-Cys-Asp-Xaa-Xaa-Leu-Xaa-Ala- | [SEQ ID NO: 5] |
| Xaa-Asp-Leu-Cys; | |
| Asp-Leu-Val-Xaa-Leu-Gly-Leu-Glu-Cys-Trp; | [SEQ ID NO: 6] |
| CXXGPXXXXC | [SEQ ID NO:21] |
| XXXXCXXGPXXXXCXXXX | [SEQ ID NO:22] |
| CXXXXXXCXXXXXXCCXXXCXXXXXXC | [SEQ ID NO:23] |
| CCXXXCXXXXXXC | [SEQ ID NO:24] |
| CCXXXXXCXXXXCXXXXCC | [SEQ ID NO:25] |
| CXCXXXXXXXCXXXCXXXXXX | [SEQ ID NO:26] |
| XXXXXXDXCLPXWGCLWXXXX | [SEQ ID NO:155] |
| XXXXDXCLPXWGCLWXXX | [SEQ ID NO:156] |
| D X C L P X W G C L W | [SEQ ID NO:423] |
| X X X X D I C L P R W G C L W X X X, | [SEQ ID NO:424] |
| X X X X X D I C L P R W G C L W X X X X | [SEQ ID NO:425] |
| X X E M C Y F P G I C W M X X | [SEQ ID NO:426] |
| X X D L C L R D W G C L W X X | [SEQ ID NO:427] |

wherein X is any amino acid residue.

5. The conjugate molecule according to claim 1, wherein the SABM comprises any one of the amino acid sequences selected from the group consisting of SEQ ID NOs: 7-20, 27-154 and 157-421.

6. The conjugate molecule according to claim 1, wherein, the SABM comprises the amino acid sequence selected from the group consisting of: SEQ ID NOs: 7, 8, 9, 10, 11, 12, 13, 14, 15, 16, 17, 18, 19 and 20.

7. The conjugate molecule according to claim 1, wherein the SABM comprises any one of the peptides sequences described in Tables 1-9.

8. The conjugate molecule according to claim, 1, wherein the SABM binds to serum albumin with a K_{d} that is about 100 μM or less.

9. The conjugate molecule according to claim 1, wherein the TA is an antibody.

10. The conjugate molecule according to claim 9, wherein the antibody is a Fab, F(ab)₂, scFv or a diabody.

11. The conjugate molecule according to claim 1, wherein the TA binds to a cell surface protein that is elevated in a cancer.

12. The conjugate molecule according to claim 11, wherein the cell surface protein is HER2, PSMA, PCMA, KDR and Fit-1.

13. The conjugate molecule according to claim 11, wherein the cell surface protein is a B cell surface marker.

14. The conjugate molecule according to claim 11, wherein the cell surface protein is a B cell surface marker is CD20 or BR3.

15. The conjugate molecule according to claim 1 wherein the TA is an anti-HER2 antibody.

16. The conjugate molecule according to claim 15, wherein the TA is an antibody having a VH and VL sequence of SEQ ID NO:428 and 429.

17. The conjugate molecule according to claim 15, wherein the TA comprises a variant sequence of the anti-HER2 antibody that comprises SEQ ID NO:428 and SEQ ID NO:429.

18. The conjugate molecule according to claim 1, wherein the cytotoxic agent is monomethylauristatin (MMAE).

19. The conjugate molecule according to claim 1, wherein a linker moiety located between said SABM and targeting agent or cytotoxic agent is GGGS.

20. The conjugate molecule according to claim 1, wherein the SABM binds to human albumin.

21. A composition comprising the conjugate molecule according to claim 1 admixed with a pharmaceutical carrier for therapeutic use.

22. A method for reducing the toxicity of a therapeutic agent comprising the step of producing a therapeutic agent with a serum albumin binding moiety (SABM) conjugated to the therapeutic agent.

23. A method for reducing the toxicity of a therapeutic agent in a mammal comprising administering to the mammal a therapeutically effective amount of the conjugate molecule according to claim 1.

24. The method according to claim 22 or 23, further comprising the step of measuring the toxicity of the therapeutic agent:SABM conjugate in vivo.

25. A method of treating a cancer in a mammal comprising the step of treating a mammal having the cancer with a therapeutically effective amount of a conjugate molecule according to claim 1.

26. A method of treating an autoimmune disorder in a mammal comprising the step of treating a mammal having the autoimmune disorder with a therapeutically effective amount of a conjugate molecule according to claim 1 that binds to B-cells that contribute to or cause the autoimmune disorder.

27. An article of manufacture comprising a container, a composition within the container comprising a conjugate molecule according to claim 1, a package insert containing instructions to administer a therapeutically effective dose.
